(19) United States
(12) Patent Application Publication
(10) Pub. No.: US 2013/0029334 A1
(43) Pub. Date: Jan. 31, 2013

(54) mRNA As Biomarkers For Liver Injury or Other Liver Perturbations

(71) Applicants: Russell S. Thomas, Durham, NC (US); Barbara A. Wetmore, Raleigh, NC (US)

(72) Inventors: Russell S. Thomas, Durham, NC (US); Barbara A. Wetmore, Raleigh, NC (US)

(21) Appl. No.: 13/580,492

(22) PCT Filed: Mar. 2, 2011
(86) PCT No.: PCT/US11/26774

§ 371 Date: Aug. 22, 2012

Related U.S. Application Data
(60) Provisional application No. 61/311,951, filed on Mar. 9, 2010.

(51) Int. Cl. C12Q 1/68

(52) U.S. Cl. 435/6.11

(57) ABSTRACT

The present invention provides a method for assessing the likelihood or for detecting the presence of liver perturbation in an individual. The method is particularly useful for detecting drug-induced liver injury (DILI) or other forms of hepatotoxicity or hepatic perturbation. The method comprises measuring the level of at least one RNA biomarker comprising mRNA and comparing the measured level to an appropriate reference value for the sample type and RNA biomarker type, and wherein a significant difference between the measured level and the reference value is indicative of liver perturbation.

### TECHNICAL FIELD

The present invention relates, in general, to the identification of RNA biomarkers in a body fluid of individuals having liver injury or other perturbation of the liver and, in particular, to a method of detecting the existence (diagnosis) of liver injury or other liver perturbation. The invention also relates to a panel of RNA biomarkers comprising mRNA for diagnosis of liver perturbation such as liver injury or liver cell response to perturbation such as by exposure to a drug.

### BACKGROUND

A number of agents can cause serious liver injury. These include infectious agents, drugs, toxins, natural products, herbs, immune reactions, and neoplasms. Of the processes causing liver damage (“hepatotoxicity”), drug induced liver injury (“DILI”) remains a problem in healthcare. More than 900 drugs have been implicated in causing liver injury, with DILI being responsible for 5% of all hospital admissions, and 50% of all acute liver failures. Further, DILI is the most common reason for a drug to be withdrawn from the market. Many classes of drugs have been associated with DILI, with antimicrobials (e.g., anti-bacterial agents, anti-fungal agents, tuberculostatic agents) and central nervous system (CNS) agents (e.g., anti-depressants, anti-seizure agents, skeletal muscle relaxants, and analgesics), being the most common. Of the dietary supplements causing DILI, compounds that claim to promote weight loss and muscle building accounted for nearly 60 percent of the cases.

Nonlimiting examples of drugs, reported to be associated with drug-induced liver injury, include nonsteroidal anti-inflammatory drugs (NSAIDS) such as diclofenac, aspirin, phenylbutazone, sulindac, indomethacin, acetaminophen, and others; antimicrobial agents such as isoniazid, cephalexin, co-trimoxazole, amoxicillin, flucloxacillin, ciprofloxacin, erythromycin, rifampicin, and others; muscle relaxants such as tamsulosin, tizanidine, and others; CNS agents such as phenytoin, mirtazapine, benzodiazepine, 1,4 butanediol, kavalactones, Gabapentin, tizanidine, and others; and antineoplastic agents such as methotrexate, paclitaxel, and others. Clinical assessment of liver injury remains a major challenge in medicine due to a lack of reliable tests. Conventional methods available for detection of liver injury include monitoring levels of hepatic enzymes such as AST/serum glutamic oxaloacetic transaminase and ALT/serum glutamate pyruvate transaminase. These enzymes are normally found in liver cells, and are released when liver cells undergo injury. However, the ability to detect these enzymes in blood as early indicators for liver injury or as a measure of liver function is confounded, for example, by (a) elevation of these enzymes following damage to tissues other than liver (e.g., heart damage), (b) elevation of these enzymes following liver injury of a reversible or clinically insignificant nature, (c) the time to release the enzymes into the blood, and (d) limits on sensitivity of detection.

### SUMMARY OF THE INVENTION

The present invention relates generally to the use of one or more RNA biomarkers comprising messenger RNA (“mRNA”) as a biomarker for detection, and characterization, of liver perturbation such as liver injury or induction of a canonical pathway in the liver as a result of drug exposure. More specifically, the invention demonstrates elevated levels of mRNA biomarkers found in body fluid from individuals in a rodent model of liver injury and, in a correlative manner, with elevated levels of mRNA biomarkers found in body fluid from human individuals (“patients”) having DILI. Thus, in one aspect of the invention, demonstrated is the utility that an animal model may be used as a standard in vivo model for identifying mRNA biomarkers for detection of liver perturbation in humans. In another aspect of the invention, mRNA biomarkers found in a biological sample, such as a body fluid or processed fraction thereof (e.g., blood (whole blood, or blood fractions such as serum, plasma, exosomes, microvesicles, or other microparticles (as described herein in more detail) may be used to detect liver perturbation such as liver injury. In one method of the invention provided is a method for detecting liver perturbation in an individual, the method comprising: (a) detecting, in a biological sample obtained from the individual, the level of one or more (i.e., at least one) RNA biomarkers comprising mRNA (“mRNA biomarkers”); (b) comparing the level of one or more RNA biomarkers from step (a) with a reference value for each of the one or more RNA biomarkers from step (a) (e.g., the reference value being obtained by detecting, in one or more samples, the level of one or more RNA biomarkers from individual(s) lacking liver perturbation); whereby a difference (e.g., the difference being either an increase or a decrease, depending on the particular mRNA biomarker being detected) in the level of the one or more RNA biomarkers comprising mRNA as compared to the reference value for the one or more RNA biomarker indicates the presence of or likelihood of (is indicative of) a liver perturbation.

Some drugs are inducers of liver enzymes, which is another example of perturbation of the liver. Shown herein is that transcriptional activation is another result of drug exposure that is measurable by a method for detecting perturbation of the liver according to one aspect of the invention. For example, certain anticonvulsant drugs and antibiotics are known to be potent inducers of cytochrome P-450 induction (e.g., rifampicin, carbamazepine, phenobarbital, phenytoin, primidone, and others). Thus, in another aspect of the invention, provided is a method of detecting liver enzyme induction, the method comprising: a) detecting, in a biological sample obtained from the individual, a level of one or more RNA biomarkers comprising mRNA (mRNA biomarkers); (b) comparing the level of one or more RNA biomarkers from step (a) with a reference value for the one or more RNA biomarkers (e.g., the reference value being obtained by detecting, in one or more control samples the level of one or more RNA biomarkers from individual(s) lacking induction of liver enzymes); whereby detecting an increase in the level of the one or more RNA biomarkers as compared to the reference value is an indicator of liver enzyme induction.

In another preferred embodiment, the mRNA detected in the method of the invention is an mRNA biomarker selected from the group consisting of mRNA biomarkers listed in Table 6, Table 8, and a combination thereof. Also provided is a panel of mRNA biomarkers indicative of liver perturbation caused by a drug, or by more than one drug of the same class (“class of drugs”), which is different from (e.g., not sharing common mRNA biomarkers with) a different drug or different class of drugs, respectively, as will be more evident from the description herein. In a preferred embodiment, a preferred group of mRNA biomarkers may be used to the exclusion of other mRNA biomarkers, in constructing a diagnostic panel of mRNA biomarkers indicative of liver perturbation caused by a particular drug or particular drug class (the latter being where an mRNA biomarker is common for more than one drug in the drug class).

Objects and advantages of the present invention will be clear from the description, including the figures and illustrative examples that follow.

### BRIEF DESCRIPTION OF THE DRAWINGS

FIG. 1A is a graph depicting copy numbers per mL plasma of cell-free plasma mRNA, detected as cDNA, for albumin (“Alb”) mRNA (5′ region, mid region, and 3′ region; SEQ ID NO:1) for a group of rats not treated with D-(+)-galactosamine (“DGAL”) (for determining a reference value, and as an assay control) (solid black boxes), and for a group of rats treated with 1000 mg/kg DGAL (hatched boxes).

FIG. 1B is a graph depicting copy numbers per mL plasma of cell-free plasma mRNA, detected as cDNA, for fibrinogen beta chain (“Fgb”) RNA (5′ region, mid region, and 3′ region; SEQ ID NO:2) for a group of rats not treated with DGAL (solid black boxes) for determining a reference value, and for a group of rats treated with 1000 mg/kg DGAL (hatched boxes).

FIG. 1C is a graph depicting copy numbers per mL plasma of cell-free plasma mRNA, detected as cDNA, for haptoglobin (“Hp”) RNA (5′ region and 3′ region; SEQ ID NO:3) for a group of rats not treated with DGAL (solid black boxes) for determining a reference value, and for a group of rats treated with 1000 mg/kg DGAL (hatched boxes).

FIG. 2 is a graph showing a log2 fold change in expression of Alb, Fgb, and Hp mRNA (detected as cDNA), respectively, in liver of rats treated with DGAL, or with acetaminophen (APAP), as compared to references values determined from rats not treated with DGAL (Control; solid black boxes) or APAP (Control; solid black boxes), respectively.

FIG. 3A is a graph depicting ALT serum enzyme levels in patients suspected or confirmed of having drug-induced liver injury (DILI) as compared to study control individuals (“Cont”).

FIG. 3B is a graph depicting AST serum enzyme levels in patients suspected or confirmed of having DILI, as compared to levels in study control individuals (“Cont”).

FIG. 4A is a graph depicting copy numbers per mL plasma of cell-free plasma mRNA, detected as cDNA, for albumin (“Alb”) mRNA (SEQ ID NO:4) in patients suspected or confirmed of having DILI, as compared to a reference value from study control individuals (“Cont”).

FIG. 4B is a graph depicting copy numbers per mL plasma of cell-free plasma mRNA, detected as cDNA, for fibrinogen beta chain (“Fgb”) mRNA (SEQ ID NO:5) in patients suspected or confirmed of having DILI as compared to a reference value from study control individuals (“Cont”).

FIG. 4C is a graph depicting copy numbers per mL plasma of cell-free plasma mRNA, detected as cDNA, for haptoglobin (“Hp”) mRNA (SEQ ID NO:6) in patients suspected or confirmed of having DILI as compared to a reference value from study control individuals (“Cont”).

### DETAILED DESCRIPTION OF THE INVENTION

The present invention relates to a method of detecting the presence or absence of liver injury, or other liver perturbation (e.g., enzyme induction, or transcriptional activation, induction of a canonical pathway in the liver, or other change in liver physiology), in an individual by measuring the level of one or more RNA comprising mRNA biomarkers in a sample obtained from the individual. The level of the one or more mRNA biomarkers is compared to a reference value for that type of mRNA biomarker measured, and a significant difference between the measured level of one or more mRNA biomarkers and the reference value for the measured mRNA biomarker is an indicator of liver perturbation.

Cellular release of RNA molecules into the circulation can occur through multiple mechanisms. Among passive processes, the release of cellular mRNA and miRNA has been shown following necrotic cell death. The RNA molecules enter circulation and are either associated with cellular debris or in naked form. Among active processes, mRNA and miRNA molecules have been identified within membrane-encapsulated vesicles released by cells. These include exosomes, shedding vesicles, and apoptotic blebs. Exosomes are small vesicles (40-100 nm) that are formed by inward budding of endosomal membranes. The vesicles are packaged within larger intracellular multivesicular bodies that release their contents to the extracellular environment through exocytosis. Shedding vesicles (<200 nm) are released from live cells through direct budding from the plasma membrane, while apoptotic blebs (100->1000 nm) bud directly from the plasma membrane upon cell death. Some of the vesicles move by diffusion from the extracellular space into the circulation and appear in biological fluids. Many pathological conditions and cellular perturbations can stimulate further release of the particles containing RNA molecules.

The present invention shows that use of an RNA comprising mRNA found in a biological sample as a biomarker offers several advantages. First, it can be readily obtained from individuals suspected of having liver perturbation. Second, amplification technologies such as polymerase chain reaction (“PCR”) allow highly sensitive and quantitative detection of specific mRNAs. Third, identification of targets of toxicity can be achieved using tissue-specific transcripts. Finally, microarray technologies can be exploited to broadly survey transcriptional changes in biological processes and signaling pathways and develop high-dimensional transcriptional profiles to discriminate among disease states, treatments, or perturbation of the liver. This latter characteristic enables identification of etiologies of liver perturbation (e.g., liver injury or liver enzyme induction or induction of a canonical pathway in the liver) to the extent that different causative processes (and different drugs) may generate distinct transcriptional profiles, and distinct profiles of mRNA biomarkers detectable in a biological sample, wherein such profiles may be used in a diagnostic panel.

### DEFINITIONS

While the following terms are believed to be well understood by one of ordinary skill in the art of biotechnology, the following definitions are set forth to facilitate explanation of the invention.

The term “RNA biomarker” or “mRNA biomarker”, as used herein, means any RNA polynucleotide comprising mRNA, or a fragment thereof, having a sequence that is transcribed from DNA within a hepatocyte or other cell type found in the liver (e.g., immune cell, or endothelial cell), and is measured as mRNA or as cDNA derived therefrom (including a fragment or portion thereof of from about 10 bases to about 100 bases). The mRNA biomarker may further include processing, following being copied or transcribed from DNA, such as capping, splicing, and/or polyadenylation; or reverse transcription to cDNA which may then be subjected to amplification, fragmentation, and quantitation using methods well known in the art. In a preferred embodiment, mRNA biomarker is reverse transcribed into cDNA, and with amplification (e.g., using any one of commercially available kits) so as to improve sensitivity of detection of the mRNA biomarker's presence in the biological sample from which it was derived. In a preferred embodiment, preferred mRNA biomarkers may be used in the compositions and methods of the invention to the exclusion of other circulating mRNA (such as any that have been previously described in the art).

The term “reference value”, as used herein, means a standard or assay control value that is determined from (a) healthy (not suspected or known to have liver perturbation) individual (‘s/s’) biological sample of the same tissue or fluid type as that being assayed, or from which the mRNA is derived, from an individual suspected of having a liver perturbation, and as measured for the same kind of mRNA biomarker being detected in or from a biological sample obtained from an individual suspected of having liver perturbation.

The term “liver injury”, as used herein, means any type of hepatotoxicity including, but not limited to, drug-induced livery injury, inflammation, degeneration or other hepatotoxicity caused by agents other than drugs (e.g., infectious agents, toxins, natural products, or disease processes (e.g., cancer or immune-mediated)). In a preferred embodiment, a type of liver injury which is preferred to be detected according to the method of the present invention, and to the exclusion of other types of liver injury in this preferred embodiment, comprises drug induced liver injury (DILI) caused by one or more drugs selected from the classes of drugs comprising NSAIDs, antimicrobials, central nervous system agents, muscle relaxants, and antineoplastic agents.

The term “liver perturbation”, as used herein, means one or more of: any type of liver injury; induction of one or more liver enzymes, or induction of a canonical pathway listed in Tables 6 and 7 herein (as measured by a difference in the level of an mRNA biomarker associated with that canonical pathway as compared to a reference value; see, e.g., Tables 6 and 7); as a result of exposure to a drug. Canonical pathway is used herein to mean a physiological, biological (including but not limited to metabolic, cellular, immunologic, hematologic), or chemical process that is known or thought to occur in the liver.

The term “liver enzymes”, as used herein, means enzymes produced by hepatocytes or other cell types found in the liver. These include, but are not limited to, ALT, AST, alkaline phosphatase, bilirubin, sorbitol dehydrogenase (SDH), and one or more cytochromes (e.g., one or more of the family of P450 cytochromes, or other cytochromes) found in the liver.

The terms “sample” or “biological sample” are used interchangeably herein to mean a body fluid such as blood, or blood products such as serum, plasma or the like, or other body excretion or secretion such as saliva, urine, lymph, bile, feces, sweat, or breath vapor. Each of these specific examples of types of body fluids may comprise a type of biological sample.

The term “difference” is used herein, when referring to a comparison between (i) a level of the one or more RNA biomarkers comprising mRNA measured or derived from a biological sample obtained from an individual having or suspected of having liver perturbation (“test sample”) to (ii) a corresponding reference value (e.g., “corresponding” means that the reference value was determined or derived from the same type of biological sample and same species of mRNA detected with respect to the test sample), to mean a measurable difference, wherein typically the difference exceeds a predetermined threshold. For example, the predetermined threshold can be represented using one or more mathematical parameters (e.g., geometric mean) or statistical parameters (e.g., a standard deviation). In a preferred embodiment, a difference between the level of an mRNA detected herein as an RNA biomarker of liver perturbation (including, but not limited to, liver injury or hepatotoxicity) is at least 2 fold, and more preferably greater than 2 fold (e.g., about 10 fold or 15 fold or 20 fold or 50 fold or 100 fold or more) as compared to the reference value or corresponding reference value.

Certain aspects of the invention can be described in greater detail in the non-limiting Examples that follows.

### Example 1

In this example, to model a drug causing liver perturbation (such as injury or hepatotoxicity), hepatotoxicant D-galactosamine (“DGAL”) was administered to rats. Male Sprague-Dawley rats were administered DGAL at 0 (sterile PBS) or 1000 mg/kg intraperitoneally, and sacrificed after 24 hours. Histologically-stained sections of liver from rats treated with DGAL showed that DGAL induced moderate panlobular hepatocellular necrosis that was randomly distributed throughout the liver. The necrosis was observed in all of the treated animals, which were graded histologically as 3 on a scale of 0 to 5. Blood was harvested from DGAL-treated rats, and serum liver enzymes were measured using a standard laboratory assay. As shown in Table 1, DGAL induced treatment-related increases in serum ALT and AST levels. Values are Mean±SEM. n=15 rats per group. ***p<0.001, Welch's ANOVA F-test. Statistically significant, treatment-related increases were observed for serum ALT and AST levels, with increases of 109 and 81-fold over controls, respectively

TABLE 1

| | |
|---|---|
| Serum | DGAL Treatment (mg/kg) |

| Enzyme^{a} | 0 | 1000 |
|---|---|---|
| ALT | 43.20 ± 1.89^{b} | 4712.47 ± 621.49*** |
| AST | 102.20 ± 4.32 | 8228.07 ± 852.46*** |
| DBIL | 0.00 ± 0.00 | 0.32 ± 0.08*** |
| IBIL | 0.20 ± 0.00 | 0.57 ± 0.08*** |
| TBIL | 0.20 ± 0.00 | 0.89 ± 0.15*** |

Abbreviations and units are as follows:
ALT: alanine aminotransferase, U/L;
AST: aspartate aminotransferase, U/L;
DBIL: direct bilirubin, mg/dl;
IBIL: indirect bilirubin, mg/dl; and
TBIL: total bilirubin, mg/dl.

RNA was isolated from the cell-free plasma of DGAL-treated rats using a commercially available RNA isolation kit, and mRNA was reversed transcribed using a commercially available kit, with resultant cDNA being amplified using a commercially available universal polymerase chain reaction (PCR) master mix (Taqnnan) with probes and primers, and subjected to qPCR analysis. Taqnnan gene expression assays targeting the 5′, middle and/or 3′ regions were analyzed. Standard curves were generated in all of the assays and absolute quantitation used to determine copy number per mL plasma. Copy number per mL plasma was calculated based on standard curves generated from plasmid DNA. Plasmid DNA was prepared using cDNA clones obtained commercially (Open Biosystems, Huntsville, Ala.) and purified using a plasmid purification kit. Clone information is as follows: Alb (Open Biosystems clone ID #7303856); Fgb (Open Biosystems clone ID#7371665); Hp (Open Biosystems clone ID#7321960); and Actb (Open Biosystems clone ID#6920838).

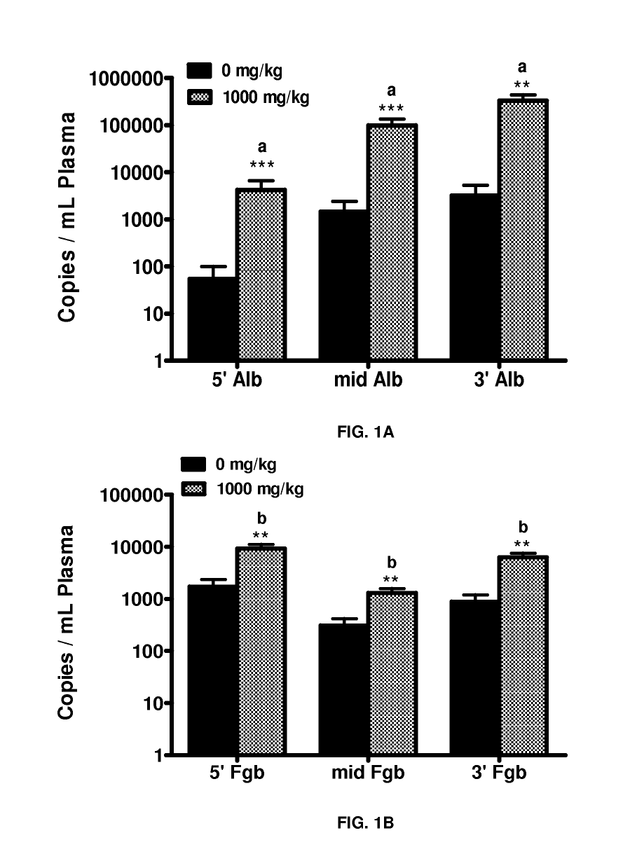

As shown in FIGS. 1A, B, and C, DGAL induces treatment-related increases in circulating albumin mRNA (Alb; FIG. 1A; SEQ ID NO:1), fibrinogen beta chain mRNA (Fgb; FIG. 1B; SEQ ID NO:2) and haptoglobin mRNA (Hp; FIG. 1C; SEQ ID NO:3) levels. Statistically significant treatment-related increases were observed for all regions of all of the mRNA tested. Values are Mean±SEM, n=8 rats per group. **p<0.01; ****p<0.0001; as determined by either the two-tailed t-test or by Welch's ANOVA F-test.

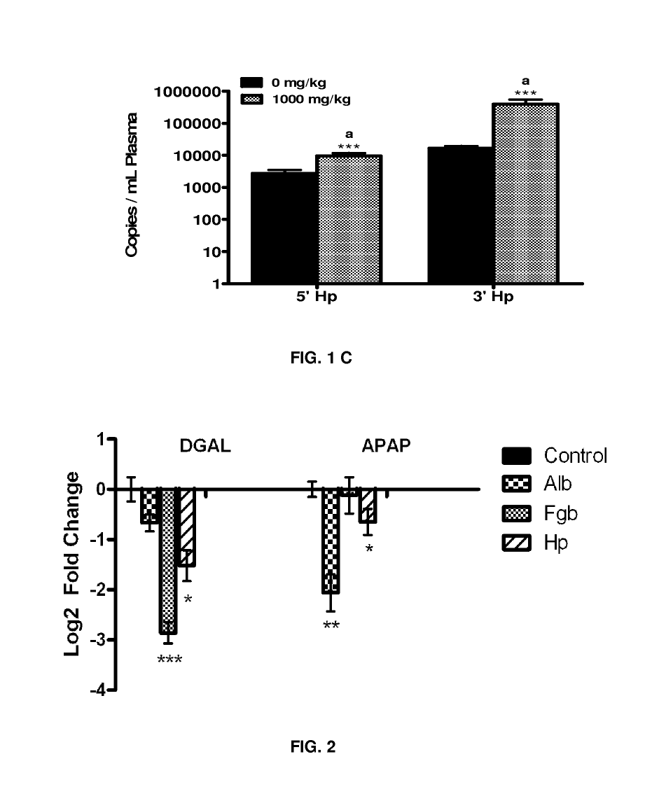

Given potential concerns regarding the integrity of RNA circulating in the blood where RNase levels are known to be high, multiple regions of the mRNA from each of the genes were interrogated (see Table 2). Assays directed towards the 5′, mid and 3′ regions of Alb demonstrated fold increases of 78, 68 and 103, times that of reference (control) values for Alb, respectively. Assays directed against the 5′, mid and 3′ regions of Fgb demonstrated fold increases of 5.4, 4.2, and 7.1 times that of reference values for Fgb, respectively. Lastly, assays directed against the 5′ and 3′ regions of Hp demonstrated fold increases of 3.5 and 24 over reference values for Hp, respectively. For all three RNA biomarkers, treatment-related increases were observed in all of the regions tested of mRNA. Notably and surprisingly, these increases in circulating Alb, Fgb, and Hp mRNAs occurred despite a significant decrease in the expression of Fgb and Hp mRNAs in the livers of treated animals (FIG. 2). In addition, a small but statistically insignificant decrease in Alb mRNA was observed in the livers of treated animals.

TABLE 2
Gene Expression Assay Information for qPCR Analyses

| | Region | Accession No. | SEQ ID NO: | Exon Boundary |
|---|---|---|---|---|
| Alb | 5′ | NM_134326.2 | 1 | 1-2 |
| | mid | | | 6-7 |
| | 3′ | | | 12-13 |
| Fgb | 5′ | NM_020071.2 | 2 | 2-3 |
| | mid | | | 4-5 |
| | 3′ | | | 7-8 |
| Hp | 5′ | NM_012582.2 | 3 | 2-3 |
| | 3′ | | | 5-5 |
| Controls | | | | |
| Actb | 3′ | NM_031144.2 | 22, rat | 4-5 |
| GAPDH | 5′ | NM_017008.3 | 72, human | 3-3 |
| | | | 74, rat | |
| | | | 76, human | |

### Example 2

In this example, to exemplify a drug causing liver injury, drug acetaminophen (“APAP”) was administered to rats. Rats were administered APAP at 0, 100, 700, and 1400 mg/kg by gavage, and then sacrificed at 6, 24 or 48 hours after treatment. Hematoxylin and eosin-stained liver sections were examined by an accredited pathologist and scored for incidence and severity of hepatocellular necrosis. No evidence of hepatotoxicity was evident 6 hours after treatment at any of the doses. At 24 hours, hepatotoxicity was observed at 700 and 1400 mg/kg. Moderate hepatocellular necrosis was observed in the centrilobular region at 700 mg/kg in two of the eight animals examined. At 1400 mg/kg, extensive centrilobular necrosis was observed in all eight of the treated animals. In many cases, the coagulative necrosis bridged into the centrilobular regions of adjacent lobules. At 48 hours, one rat from each of the 700 and 1400 mg/kg treatment groups died. In all of the surviving rats, moderate to moderately severe hepatocellular necrosis was observed. Similar to the 24 hour time point, coagulative and bridging necrosis was observed. There was no histologic evidence of APAP-related hepatotoxicity at 100 mg/kg at any of the time points.

Using methods described previously herein, liver enzymes including serum ALT and AST levels, and plasma mRNA levels, were also assessed to determine hepatotoxicity following APAP administration. As shown in Table 3, APAP treatment increased circulating Alb, Fgb and Hp levels in a dose and time-dependent manner. No treatment-related increases were observed 6 hours after APAP treatment.

TABLE 3
Serum Enzyme Activities following APAP Treatment

| | Serum | APAP Treatment (mg/kg)^{a} | | | |
|---|---|---|---|---|---|
| | Enzyme^{b} | 0 | 100 | 700 | 1400 |
| 6 hr | ALT | 45.38 ± 3.97 | 38.25 ± 4.29 | 45.88 ± 2.95 | 50.88 ± 3.54 |
| | AST | 113.63 ± 6.65 | 101.38 ± 7.46 | 137.13 ± 10.99 | 142.63 ± 5.55 |
| | DBIL | 0.00 ± 0.00 | 0.00 ± 0.00 | 0.00 ± 0.00 | 0.00 ± 0.00 |
| | IBIL | 0.10 ± 0.00 | 0.11 ± 0.01 | 0.10 ± 0.00 | 0.11 ± 0.01 |
| | TBIL | 0.10 ± 0.00 | 0.11 ± 0.01 | 0.10 ± 0.00 | 0.11 ± 0.01 |
| 24 hr | ALT | 55.63 ± 4.42 | 67.25 ± 8.95 | 159.00 ± 74.04 | 2372.38 ± 984.27^{c} |
| | AST | 122.88 ± 9.06 | 146.38 ± 16.85 | 433.00 ± 199.82 | 12637.25 ± 5116.21^{c} |
| | DBIL | 0.00 ± 0.00 | 0.00 ± 0.00 | 0.01 ± 0.01 | 0.09 ± 0.02^{c} |
| | IBIL | 0.10 ± 0.00 | 0.10 ± 0.00 | 0.13 ± 0.02 | 0.19 ± 0.05 |
| | TBIL | 0.10 ± 0.00 | 0.10 ± 0.00 | 0.14 ± 0.02^{c} | 0.28 ± 0.06^{c} |
| 48 hr | ALT | 55.75 ± 4.36 | 57.25 ± 4.68 | 803.00 ± 225.80^{d} | 2050.00 ± 674.71^{d} |
| | AST | 120.50 ± 6.10 | 118.88 ± 8.02 | 1225.43 ± 324.27^{d} | 3990.33 ± 1260.00^{d} |
| | DBIL | 0.00 ± 0.00 | 0.00 ± 0.00 | 0.04 ± 0.02^{d} | 0.12 ± 0.03^{d} |
| | IBIL | 0.16 ± 0.02 | 0.13 ± 0.02 | 0.17 ± 0.02 | 0.18 ± 0.04 |
| | TBIL | 0.16 ± 0.02 | 0.13 ± 0.02 | 0.21 ± 0.03^{d} | 0.30 ± 0.07^{d} |

^{a}Mean ± SEM, n = 7-8 rats per treatment group.
^{b}See Table 1 for abbreviation and unit definitions.
^{c}p < 0.05, vs. 24 hr control (Kruskal-Wallis test).
^{d}p < 0.05, vs. 48 hr control (Kruskal-Wallis test).

APAP treatment increased circulating Alb, Fgb and Hp mRNA levels in a dose- and time-dependent manner. No treatment-related increases were observed 6 hours after APAP treatment. At 24 hours, statistically significant increases in circulating levels were observed for all three liver-specific RNA biomarkers comprising mRNA as compared to the respective reference values for those mRNA biomarkers. In that regard, circulating Fgb and Hp mRNA were significantly increased at 100, 700, and 1400 mg/kg APAP, with Fgb mRNA levels exhibiting fold increases of 6, 61, and 131, over respective reference values. Hp increased by 39, 230 and 158 fold over reference values, respectively. Circulating Alb mRNA was significantly increased by 1900 and 875 fold over reference values, respectively, at 700 and 1400 mg/kg. At 48 hours, Hp mRNA levels were increased by 9, 54 and 86 fold over controls, respectively, at 100, 700, and 1400 mg/kg APAP; while Alb mRNA levels were increased by 13 and 31 fold at 700 and 1400 mg/kg. Circulating Fgb mRNA levels were significantly increased by 5 and 13 fold over controls, respectively, at 700 and 1400 mg/kg. A surprising observation, similar to that observed with DGAL, is that the increase in circulating Alb, Fgb, and Hp mRNAs in the 1400 mg/kg treatment group occurred despite a significant decrease in the expression of Alb and Hp mRNAs in the livers of treated animals (FIG. 2).

### Example 3

Shown in this example is that mRNA detection in a biological sample can be liver-specific (e.g., caused by liver perturbation such as liver injury, hepatotoxicity, liver enzyme induction, induction of a canonical pathway in the liver, or a combination thereof). Rats were treated with skeletal muscle toxicant bupivacaine (“BPVC”). For BPVC treatment, rats were administered 0.5 mL sterile saline or 0.5% w/v sterile solution of BPVC in saline once into both the right and left tibialis anterior muscles. Using the general methods described in Examples 1 and 2 herein, liver enzymes and mRNAs (inducible in the liver and capable of circulating in body fluid) were measured from BPVC-treated rats, and compared to controls animals. To compare the specificity of circulating liver mRNAs with AST and ALT following skeletal muscle injury, rats were treated with BPVC, and then serum enzymes and circulating liver mRNAs were measured 24 hours after treatment. BPVC treatment induced a modest, but statistically significant elevation in serum ALT (1.96×) and AST (3.58×) levels (Table 4). However, plasma Alb, Fgb, and Hp mRNA levels remained unchanged with BPVC treatment. This demonstrates that the method of the present invention which measures RNA biomarkers in biological samples provides a greater specificity in detecting hepatotoxicity than possible with serum transaminases.

TABLE 4
Serum Enzyme Activities following BPVC Treatment

| | |
|---|---|
| Serum | BPVC Treatment |

| Enzyme^{a} | 0 | 0.5% |
|---|---|---|
| ALT | 37.60 ± 1.11^{b} | 73.70 ± 4.69**** |
| AST | 102.80 ± 7.03 | 368.25 ± 31.81**** |

^{a}See Table 1 for abbreviation and unit definitions.
^{b}Mean ± SEM, n = 10 rats per control group, 20 rats per treatment group. Statistically significant differences are listed in bold.
****p < 0.0001, as determined by Welch's ANOVA F-test.

### Example 4

In this example, shown is separation of plasma by sucrose-density gradient centrifugation, revealing density-specific mRNA distribution and microparticle profiles following DGAL treatment. Under normal conditions, naked mRNAs should be rapidly degraded in blood, and if so, may not serve as a robust biobiomarker of liver injury. To examine the form of the circulating liver mRNAs and the mechanism protecting them from degradation, the 14,000×g pellets isolated from the plasma of control and DGAL-treated rats were separated by sucrose density gradient centrifugation. Electron microscopic (EM) and qPCR analyses were performed on each fraction to determine the size and state of the microvesicles and any density- and treatment-related variations in mRNA levels.

Plasma was thawed on ice, diluted in an equal volume of sterile PBS, treated with protease inhibitors (500 μM AEBSF HCl (4-(2-Aminoethyl)benzenesulfonyl fluoride hydrochloride), 150 nM aprotinin, 1 μM E-64, and 1 μM leupeptin hemisulfate) and centrifuged at 14,000×g for 45 minutes at 4° C. The 14,000×g pellet was resuspended in sterile PBS and spun again at 14,000×g for 45 minutes at 4° C. The pellet was immediately stored at −80° C. The separation of plasma microparticles and cellular debris by sucrose density gradient centrifugation was conducted with methods known in the art. The plasma 14,000×g pellets were resuspended in a stock solution of 20 mM HEPES, 2.5M sucrose, pH 7.4 and transferred to an ultracentrifuge tube. The sucrose density gradient was generated by layering equal volumes of 2M, then 0.25 M sucrose solutions on top of the microparticle suspension. The tube was sealed and stored horizontally for 3 hours to generate a uniform sucrose gradient. The tube was slowly brought to vertical and spun at 210,000×g in an SW 40-Ti swinging bucket rotor for 19 hours at 4° C. Eleven 1 mL fractions were collected and the refractive index measured using a refractometer to determine the density. Each fraction was then transferred to a 3 mL ultracentrifuge tube and mixed with 2 mL 20 mM HEPES solution, pH 7.4 prior to centrifugation at 110,000×g for 1 hour at 4° C. The pellet was then resuspended in sterile PBS with half of the resuspension undergoing immediate RNA isolation and the second half stored at −80° C. Two independent sucrose density gradient centrifugation experiments were conducted. Using methods described in Example 1 herein, RNA was isolated, reverse-transcribed, with cDNA being amplified and subjected to qPCR analysis.

With a few exceptions, the Alb, Fgb, and Actb mRNA were present in control animals (not treated with DGAL) in higher amounts in the mid density fractions (1.10-1.18 g/ml) when compared to the low/high density fractions. For Fgb mRNA, fractions 1.07 and 1.10 g/ml also contained relatively high amounts, while for Hp mRNA the low density fractions (1.07-1.10 g/ml) and the 1.21 g/ml fraction contained the highest amounts of mRNA. Differential distribution of the various mRNAs among the density fractions was also observed following treatment with DGAL. For Alb mRNA, treatment-related increases were observed in all fractions with the middle fractions (1.10-1.18 g/ml) containing 89% of the total mRNA copies. Similarly, Fgb mRNA showed treatment-related increases in all density fractions with the exception of the 1.10 g/ml fraction. The middle fractions also contained the highest treatment-related increases and contained 84% of the total mRNA copies. For Hp mRNA, no treatment related changes were observed in the low density fractions, while the middle fractions contained the majority of changes and contained 81% of the mRNA copies. Some treatment-related changes in Hp mRNA were also observed in the high density fractions. The distribution of Actb mRNA was similar to Fgb with treatment-related differences primarily in the middle and high density fractions.

For electron microscopic examination, pellets from selected fractions from the sucrose density gradient experiments were examined using whole mount electron microscopy as known in the art. Briefly, the microparticle pellets (previously resuspended in PBS) were fixed in 2% paraformaldehyde prior to absorption to a formvar-carbon coated EM grid. The grids were post-fixed with 1% glutaraldehyde prior to contrasting in a solution of uranyl oxalate, pH 7. The grids were further contrasted and embedded using a solution of 4% uranyl acetate and 2% methyl cellulose. The grids were then observed on an EM400 transmission electron microscope at 80 kV. Electron microscopic examination of the microparticle fractions 1, 4, 5, 6, 9 and 11 (corresponding to densities 1.07, 1.11, 1.13, 1.18, 1.24, and 1.26 g/mL) revealed that intact, spherical microparticles were present in all of the control fractions analyzed. However, DGAL-treated fractions of intermediate densities (1.11, 1.13, and 1.18 g/mL) contained debris, cell fragments and misshapen vesicles, in addition to intact spherical microparticles. DGAL-treated fractions of low density (1.07 g/ml) showed minimal cellular debris, while the high density fractions (1.24 and 1.26 g/mL) showed no evidence of debris or fragmentation. DGAL treatment caused an increase in mean microparticle diameter in certain density fractions, most notably at the ends of the gradient (1.07 and 1.25 g/mL). Cellular debris was excluded from the analysis in the assessment of microparticle size.

These combined measurements indicate that, in untreated control individuals, mRNAs were contained within microparticles, which were presumably providing protection from ubiquitous RNAses. The liver and “housekeeping” mRNAs were also not distributed uniformly through the density gradient. With the exception of Hp mRNA, the mRNAs in control individuals were generally present in higher amounts in the middle density fractions (1.10-1.18 g/ml), which are traditionally identified as exosomal. However, based on the respective size differences between exosomes (40-100 nm) and shedding vesicles (<200 nm), density fractions 1.11 and 1.13 g/ml appear to contain a significant percentage of shedding vesicles in control individuals, while the remaining fractions appear primarily exosomal. Apart from the higher amount of mRNAs in the middle fractions in control individuals, each mRNA appeared to have a unique distribution among the various density fractions. The biological significance of the different density distributions of mRNAs in the untreated individuals is unclear, but one potential explanation is that the different mRNAs may be selectively packaged into exosomes and shedding vesicles.

In liver injured individuals (e.g., following DGAL treatment), a similar non-uniform distribution of the mRNAs among the density fractions were observed, except that all mRNAs showed treatment-related increases in the middle density fractions. The treatment-related increase in the middle density fractions was primarily due to the presence of cellular debris in these fractions and the apparent nonselective release of mRNAs through cell lysis. Presumably, the association with cellular debris also protects the mRNA from rapid degradation. Interestingly, the Hp mRNA showed treatment-related increases in mRNA only in the middle fractions that correspond to the cellular debris, while Alb and Fgb mRNA also showed increases in the low and high density fractions. Given that DGAL stimulated the release of larger particles in the low and high density fractions, it seems likely that Alb and Fgb mRNAs are actively released in these particles. As known to those skilled in the art, an increased release of shedding vesicles has been shown to occur following cellular perturbation.

Collectively, the unfractionated measurements of circulating mRNAs produced from the liver following DGAL and APAP administration and the sucrose density gradient analyses provide an assessment of the nature of these mRNAs in the circulation with and without liver injury. All liver-specific and “housekeeping” mRNAs were present at detectable levels in control individuals indicating that the active release by liver cells of these RNA biomarkers comprising mRNAs in exosomes and shedding vesicles (“nnicroparticles”) is a physiological process and not dependent on overt liver injury. In treated individuals, increase in the levels of mRNA biomarkers of liver origin in the general circulation preceded pathological changes or increases in serum transaminases with respect to dose and were shown to be present in both microparticles and cellular debris. This suggests a dose- or injury-dependent shift in the mechanism of release from an active process at low, nontoxic doses to both active and passive processes at cytotoxic doses. Surprisingly, the increase in the level of circulating mRNA biomarkers of liver origin occurred despite a significant decrease in the expression of these mRNAs in the livers of treated individuals. This supports the notion that an increase in circulating liver mRNAs of liver origin is through increased release, and not through increased transcription.

In another embodiment of the present invention in which RNA biomarker comprising mRNA is isolated from the biological sample to be tested (“test sample”) in association with one or more of nnicroparticles and cellular debris, rather than separate the nnicroparticles and cellular debris by density gradients, the biological sample is subjected to a centrifugation step. The biological sample was diluted in an appropriate reaction buffer (e.g., phosphate buffered saline or other suitable buffer), treated with one or more protease inhibitors (e.g., 500 μM AEBSF-HCl, 150 nM aprotinin, 1 μM E-64, and 1 μM leupeptin hemisulfate), and centrifuged at between 14,000×g to 20,000×g. The resulting pellet, containing RNA biomarkers comprising mRNA (associated with one or more of microparticles and/or cellular debris), was resuspended in buffer, and centrifuged again. The RNA biomarkers comprising mRNA were then isolated from the pellet by a commercially available RNA isolation kit. Thus, as an optional step to include in the methods of the present invention, one or more of microparticles and cellular debris may first be isolated from a biological sample by either density gradients or by centrifugation (both embodiments described in this Example 4), from which RNA biomarkers comprising mRNA are subsequently measured. Alternatively, the RNA biomarkers comprising mRNA were isolated and measured from, or measured directly in, the biological sample by using RNA isolation and/or mRNA detection methods described herein or as known to those skilled in the art.

It is within the purview of one skilled in the art to design and select the appropriate primers, probes, and enzymes for RNA isolation and mRNA detection methods. For example, nucleic acid primers and nucleic acid probes for a specific mRNA biomarker can be selected or derived from the sequence of that mRNA biomarker or its cDNA, for instance as can be derived from Examples 5 and 6 herein, and the accompanying Sequence Listing. Likewise, depending on the assay format used for the method of the present invention, one skilled in the art may optimize hybridization conditions for one or more of amplification or detection of an mRNA biomarker (as mRNA or its corresponding, amplified cDNA). For those assay formats in which high selectivity is desired, relatively high stringency conditions may be used in forming the nucleic acid hybrids. For example, relatively low salt (e.g., 0.02 M to about 0.10M NaCl) and/or high temperatures (of about 50° C. to about 70° C.) or other suitable conditions can be used for detecting specific mRNA transcripts comprising biomarkers. Also, it may be advantageous to incorporate an indicator in the detection of the mRNA biomarker (e.g., incorporated or coupled to a probe to the mRNA biomarker, or incorporated or coupled to amplified cDNA derived from the mRNA biomarker). Such indicators are known in the art to include fluorescent molecules (e.g., fluorescent labels or fluorophores such as the Alexa series, fluorescein isothiocyanate, Oregon green series, rhodamine series, fluorescent protein series), luminescent molecules (e.g. comprising Lanthanide and ruthenium complexes), colorimetric molecules, molecular beacons, or other molecules (e.g., avidin/biotin with subsequent enzymatic detection) which are capable of being detected. Also provided is a kit that may comprise one or more containers (e.g., vial, tube, or other suitable carrier means) each containing, or separately containing, kit components comprising primers and probes (wherein the probe may already be labeled or is capable of being labeled for detection using an indicator), and, optionally, an indicator, for measuring one or more mRNA biomarkers indicative of liver perturbation. In a preferred embodiment of the kit according to the invention, the kit components comprise such reagents (e.g., primers, probes, and the like) that enable detection of a panel of mRNA biomarkers indicative of liver perturbation that may be present in a biological sample to be tested.

### Example 5

In this example, illustrated is whole genome microarray analysis, which can be used to reveal treatment-specific transcriptomic profiles following liver injury or liver enzyme induction. Briefly, microarray analysis was performed on plasma mRNA collected from rats treated with vehicle saline or DGAL (1000 mg/kg; ip; n=6/group), and then sacrificed 24 hours later; and rats treated with vehicle 0.5% methylcellulose (n=5) or APAP (1400 mg/kg; gavage; n=3), and then sacrificed 24 hours later. Total RNA was isolated from the 14,000×g plasma pellet from rats treated with DGAL or APAP. The RNA was amplified by reverse transcription into cDNA, labeled with biotin using a commercially-available kit, and the labeled cDNA was hybridized to commercially available whole genome rat arrays (Affymetrix Rat 230_{—}2; containing DNA probe sets for hybridizing to the cDNA, wherein the sequences of the probes specific for this array are available from the Affymetrix website) using methods and conditions according to the manufacturer of the kit. More specifically, the cDNA was made from the RNA sample comprising mRNA, and the cDNA was enzymatically fragmented to form single-stranded cDNAs in the 50-100 base range. Next, this fragmented product was labeled via enzymatic attachment of a biotin-labeled nucleotide that contained an indicator molecule comprising a fluorophore. After this labeled cDNA was hybridized and bound to the oligonucleotides on the microarray, any unbound cDNA was washed away and the microarrays were scanned. During this process, the microarray chips were scanned at a wavelength that allowed the indicator molecule to fluoresce (the excitation wavelength). The level of mRNA biomarker was determined from the level of fluorescence (e.g., correlating to the amount of labeled cDNA hybridized to the microarray) using standard techniques.

Data on measurement of mRNA biomarkers (which also may be representative of gene expression induced as a result of liver perturbation) was preprocessed using GC-RMA and log₂-transformed. The probe-level microarray data was checked for quality using various graphical and statistical means. Arrays showing low quality RNA or poor amplification were excluded from the analysis. To evaluate the ability of whole genome microarray analysis using plasma mRNA to discriminate between DGAL- and APAP-induced liver perturbation, a one-way analysis of variance and individual t-tests were then used to identify which individual mRNA biomarkers were statistically altered between the two treatment groups. A false-discovery rate correction was used for multiple comparisons. Analysis software was utilized to identify networks of interacting genes and other functional groups using gene lists generated.

Using a difference comprising a 2-fold change and a false discovery rate of <0.05 as the criteria, 1374 and 804 mRNA biomarkers were identified as differentially expressed following APAP and DGAL treatment, respectively; and 132 mRNA biomarkers were shared between the DGAL treatment and APAP treatment. Of the 132 shared mRNA biomarkers, 14 were upregulated and 24 were downregulated as a result of both APAP treatment and DGAL treatment. Consequently, 38 mRNA biomarkers were similarly affected following these two treatments (i.e., were altered in the same direction). Table 6 provides information on the mRNAs biomarkers that had the greatest treatment-related increases following exposure to both APAP and DGAL, APAP alone, or DGAL alone.

TABLE 6
Treatment-related Increases in Circulating mRNA biomarkers as detected by
Microarray Analysis
Circulating mRNA biomarkers Increased in both APAP and DGAL Experiments

| mRNA | Rat | | | | | |
|---|---|---|---|---|---|---|
| biomarker | SEQ | Human* | | | | |
| detected | ID | SEQ | Accession | Fold-Change | | Pathway/ |
| as cDNA | NO: | ID NO: | No. for Rat | APAP | DGAL | Function |
| Alb | 1 | 5 | NM_134326 | 102.57 | 83.32 | inflamm. response; |
| | | | | | | acute phase response |
| | | | | | | signaling |
| Ccl2 | 7 | 8 | NM_031530 | 79.83 | 25.35 | cytokine; chemotaxis |
| Serpina1 | 9 | 10 | NM_022519 | 24.15 | 12.61 | inflamm. response; |
| | | | | | | immune cell |
| | | | | | | trafficking |
| Rbp4 | 11 | 12 | NM_013162 | 20.44 | 36.82 | retinol binding; |
| | | | | | | insulin resistance |
| G1p2^{a} | 13 | 14 | NM_001106700 | 17.16 | 5.78 | interferon induced; |
| | | | | | | ubiquitin-like |
| Mt2a | 15 | 16 | XM_001062488 | 16.40 | 18.20 | oxidative stress |
| Alb | 1 | 5 | NM_134326 | 11.26 | 77.08 | inflamm. resp.; acute |
| | | | | | | phase response |
| | | | | | | signaling; endocytosis |
| Lta4h | 17 | 18 | NM_001030031 | 6.27 | 3.40 | xenobiotic |
| | | | | | | metabolism; |
| | | | | | | arachidonic acid |
| | | | | | | metabolism |
| Gpnmb | 19 | 20 | NM_133298 | 6.10 | 4.04 | integrin pathway; cell |
| | | | | | | signaling; adhesion |
| LOC259245 | 21 | nk** | NM_001024248 | 4.94 | 11.52 | alpha-2 u globulin, |
| | | | | | | PGCL5 |
| Tuba4a | 23 | 24 | NM_001007004 | 4.21 | 2.24 | alpha tubulin 4a; |
| | | | | | | cancer |
| Pgrmc1 | 25 | 26 | NM_021766 | 2.82 | 3.12 | heme binding; |
| | | | | | | CYP450 metabolism |
| Prdx5 | 27 | 28 | NM_053610 | 2.57 | 2.03 | cell response; |
| | | | | | | modification of H₂O₂ |

*data from whole rat array; corresponding human mRNA sequence (“analog sequence”) to
mRNA biomarker measured in this standard animal model.
**nk—not known

Top 20 Circulating mRNA biomarkers Increased in APAP Experiment Only

| mRNA | Rat | | | | | |
|---|---|---|---|---|---|---|
| biomarker | SEQ | Human* | | | | |
| detected | ID | SEQ | Accession No. | | | |
| as cDNA | NO: | ID NO: | for Rat | APAP | DGAL | Pathway/Function |
| Vcl^{a} | 29 | 30 | NM_001107248 | 70.40 | | integrin signaling; |
| | | | | | | adhesion; migration |
| Cxcl2 | 31 | 32 | NM_053647 | 68.29 | | cytokine; ephrin |
| | | | | | | receptor signaling |
| Ptprj | 33 | 34 | NM_017269 | 61.43 | | hematol. System; |
| | | | | | | cell-cell signaling; |
| | | | | | | adhesion; activation |
| Hspa1a | 35 | 36 | NM_031971 | 40.60 | | inflamm. cell |
| | | | | | | recruitment; cell |
| | | | | | | death |
| Cxcl7 | 37 | 38 | NM_153721 | 37.96 | | cytokine; red blood |
| | | | | | | cell survival |
| Cxcl12 | 39 | 40 | NM_001033882 | 30.90 | | Cytokine; |
| | | | | | | hematopoiesis; |
| | | | | | | migration, apoptosis |
| Xpo5^{a} | 41 | 42 | NM_001108789 | 30.54 | | protein transport, |
| | | | | | | binding |
| Ceacam1 | 43 | 44 | NM_001033860 | 28.57 | | chemotaxis, |
| | | | | | | hematopoiesis, |
| | | | | | | apoptosis, |
| | | | | | | proliferation |
| Fam183b | 45 | 46 | XM_213312 | 26.31 | | none identified |
| Slc24a3 | 47 | 48 | XM_001054941 | 25.57 | | ion transport; calcium |
| | | | | | | homeostasis |
| Kcnab1 | 49 | 50 | NM_017303 | 24.65 | | Potassium channel |
| Pde5a | 51 | 52 | NM_133584 | 24.18 | | cyclic nucleotide |
| | | | | | | phosphodiesterase |
| Arhgap28 | 53 | 54 | NM_001191815 | 23.18 | | Rho GTPase |
| | | | | | | activating protein |
| RT1-A2 | 55 | 56 | NM_001008829 | 22.73 | | none identified |
| Slc24a3 | 57 | 58 | XM_001054941 | 22.11 | | protein transport; |
| | | | | | | cancer |
| Galnt1 | 59 | 60 | NM_024373 | 22.06 | | N-acetyl |
| | | | | | | galactosaminyl |
| | | | | | | transferase |
| Srxn1 | 61 | 62 | NM_001047858 | 21.98 | | cell-cell signaling/ |
| | | | | | | cellular response |
| St3gal2 | 63 | 64 | NM_031695 | 21.94 | | amino acid |
| | | | | | | modification |
| C2cd21 | 65 | 66 | NM_001011996 | 21.52 | | encodes |
| | | | | | | transmembrane |
| | | | | | | protein |
| Hspa1b | 67 | 68 | NM_212504 | 21.07 | | inflamm. cell |
| | | | | | | recruitment; cell |
| | | | | | | death |

*data from whole rat array; corresponding human mRNA sequence (“analog sequence”) to
mRNA biomarker measured in this standard animal model.

Top 20 Circulating mRNA biomarkers Increased in DGAL Experiment Only

| mRNA | Rat | | | | | |
|---|---|---|---|---|---|---|
| biomarker | SEQ | Human* | | | | |
| detected | ID | SEQ | Accession No. | | | |
| as cDNA | NO: | ID NO: | for Rat | APAP | DGAL | Pathway/Function |
| Ambp | 69 | 70 | NM_012901 | | 111.24 | transporter; acute |
| | | | | | | phase response |
| Mug1 | 71 | nk** | NM_023103 | | 108.00 | acute phase response |
| | | | | | | (proteinase inhibitor) |
| Cyp2c7 | 73 | nk | NM_017158 | | 102.92 | xenobiotic |
| | | | | | | metabolism |
| Obp3 | 75 | nk | NM_001033958 | | 96.26 | none identified |
| Fgg | 77 | 78 | NM_012559 | | 65.02 | hematological |
| | | | | | | system; platelet |
| | | | | | | binding |
| Ttr | 79 | 80 | NM_012681 | | 60.61 | thyroid hormone |
| | | | | | | transport, apoptosis, |
| | | | | | | acute phase response |
| Apoc3 | 81 | 82 | NM_012501 | | 58.09 | lipoprotein |
| | | | | | | metabolism; |
| | | | | | | hyperlipidemia |
| Vtn | 83 | 84 | NM_019156 | | 57.99 | cell adhesion; |
| | | | | | | spreading; protein |
| | | | | | | degradation |
| Apoa1 | 85 | 86 | NM_012738 | | 53.34 | HDL component; |
| | | | | | | cholesterol efflux; |
| | | | | | | diabetes; |
| | | | | | | hyperlipidemia, |
| | | | | | | hypercholesterolemia; |
| | | | | | | amyloidosis |
| Itih1^{a} | 87 | 88 | NM_001107291 | | 52.97 | plasma protease |
| | | | | | | inhibitor |
| Fgl1 | 89 | 90 | NM_172010 | | 52.04 | None identified |
| Kng1 | 91 | 92 | NM_001009628 | | 45.12 | proteolysis, |
| | | | | | | vascularization |
| Gc | 93 | 94 | NM_012564 | | 42.82 | None identified |
| Itih3 | 95 | 96 | NM_017351 | | 40.88 | plasma protease |
| | | | | | | inhibitor |
| Cyp2c13 | 97 | nk | NM_138514 | | 40.38 | xenobiotic |
| | | | | | | metabolism |
| Apoh | 98 | 99 | NM_001009626 | | 39.84 | lipoprotein |
| | | | | | | metabolism; |
| | | | | | | coagulation |
| Serpinc1 | 100 | 101 | NM_001012027 | | 38.19 | Serine protease |
| | | | | | | inhibitor; blood |
| | | | | | | coagulation cascade |
| Serpina3k | 102 | nk | NM_012657 | | 36.42 | Serine protease |
| | | | | | | inhibitor |
| Apoc1 | 103 | 104 | NM_001109996 | | 35.57 | Metabolic and |
| | | | | | | Endocrine Disorders; |
| | | | | | | apolipoprotein, |
| | | | | | | hyperlipidemia, |
| | | | | | | insulin resist. |
| Spp2 | 105 | 106 | NM_053577 | | 34.78 | Secreted |
| | | | | | | phosphoprotein |
| | | | | | | (cystatin) |

*data from whole rat array; corresponding human mRNA sequence (“analog sequence”) to
mRNA biomarker measured in this standard animal model.
**nk—not known

Canonical pathways involving key cellular functions that were altered in both drug treatments included cell death, hematological system development and function, and molecular transport, as shown in Table 7, and determined from using transcriptional levels of mRNA biomarkers as an indication of genes differentially expressed following drug treatment.

TABLE 7
Canonical Pathway Analysis of APAP and DGAL Microarray Data
Findings with APAP treatment

| | # genes | Key Subcategories |
|---|---|---|
| Key Functions | diff. exp. | (# genes diff. exp. in ( )) |
| Cell Death | 252 | Cell death (223)-Necrosis (21); |
| | | liver cells (18) |
| | | Apoptosis (194)-inhibition (23); |
| | | liver cells (14) |
| Hematological | 170 | Proliferation (68), |
| System Dev. and | | hematopoiesis (53), |
| Function | | differentiation (45), |
| | | adhesion (32), |
| | | chemotaxis (28) |
| Molecular Transport | 103 | Calcium flux (26), |
| | | mobilization (35), |
| | | quantity (47), |
| | | protein transport (26) |
| Immune Cell | 95 | Migration, activation, |
| Trafficking | | movement, adhesion, etc. |
| Cell Division | 91 | Arrest (47) |

| | | Gene Exp. | Selected Genes |
|---|---|---|---|
| Canonical Pathways | P value | Ratio | (Fold increase in ( )) |
| Integrin Signaling | 7.29E−08 | 31/203 | Itga6 (19); Itgb1(17); |
| | | | Vcl (70); Mapk1 (15) |
| Virus Entry via | 2.13E−06 | 17/96 | Hla-C (23); Itga6 (19); |
| Endocytic Pathways | | | Itgb1 (17); Prkc (4); |
| | | | Src (7) |
| Thrombin Signaling | 2.30E−06 | 28/203 | Arhgef12 (12); F2RI2 (12) |
| Antigen | 2.90E−06 | 9/39 | Hla-C (23); Hla-E1 (9); |
| Presentation | | | Canx (5); Calr (3) |
| Caveolar-Mediated | 3.78E−06 | 15/182 | Alb (103); Hla-C (23); |
| Endocytosis | | | Itga6 (19); Itgb1 (17); |
| | | | Itgav (12) |
| Fcy Receptor- | 4.92E−06 | 18/104 | Lyn (10); Mapk1 (15); |
| mediated | | | Nck2 (8); Src (7), |
| Phagocytosis in | | | Rac1 (4) Prkcq (3), |
| Macrophages and | | | Canx (5), Calr (3), |
| Monocytes | | | B2M (3) |
| Lipid Ag | 1.25E−05 | 6/20 | Canx (5); Pdia3 (5); |
| Presentation by | | | Psap (20); Calr (3) |
| CD1 | | | |
| Ephrin Receptor | 1.38E−05 | 25/195 | Adam10 (14); Angpt1 (23); |
| Signaling | | | Cxcl12 (31); Grin1 (10); |
| | | | Jak2 (13); Itgb1 (17) |
| B-cell Receptor | 4.14E−05 | 21/155 | Akt3 (4); Lyn (10); |
| Signaling | | | Fcgr2b (7); Dapp1 (5); |
| | | | Gsk3b (6) |

Findings with DGAL treatment

| | # genes | Key Subcategories |
|---|---|---|
| Key Functions | diff. exp. | (# genes diff. exp. in ( )) |
| Lipid Metabolism* | 83 | Metabolism (40); transport (17); |
| | | modification (22); synthesis (23) |
| Molecular | 85 | Quantity (49), transport (28); |
| Transport* | | release (25); protein localization |
| | | (10) |
| Small Molecule | 106 | Homeostasis (12); secretion |
| Biochemistry* | | (14); lipid production (14); |
| | | lipid accumulation (16) |
| Hematological | 107 | Activation (32); proliferation |
| System Dev. and | | (32); adhesion (22); chemotaxis |
| Function | | (21); coagulation (18) |
| Inflammatory | 91 | Immune response (43); inflammation |
| Response | | (27); cell movement (21); |
| | | aggregation (17); adhesion (16) |
| Cell Death | 125 | Apoptosis (104); necrosis (12) |

*lipid changes predominate (lipid metabolism, transport,
modification, etc.)

| Canonical | | Gene Exp. | Selected Genes |
|---|---|---|---|
| Pathways | P value | Ratio | (Fold increase in ( )) |
| Acute Phase | 1.07E−21 | 38/178 | Alb (83), Ambp (111), Apoa1 |
| Response | | | (53), Fgg (65), Ttr (61) |
| Signaling | | | |
| Complement | 1.05E−12 | 14/36 | C9 (10); Cfi (25); Cfb (13); |
| System | | | Cf1h (14); Serping1 (7) |
| Coagulation | 8.64E−09 | 11/37 | F2 (14); Fga (28); Fgg (65); |
| System | | | Plg (17); Serpinc1 (38) |
| Linoleic Acid | 1.54E−07 | 15/125 | Cyp2a2 (15); Cyp2c7 (103); |
| Metab. | | | |
| CYP450- | 1.90E−07 | 18/210 | Cyp2c9 (32); Cyp2c13 (40); |
| Mediated | | | Cyp3a2 (26); Pla2g4a (7)** |
| Xenobiotic | | | |
| Metab. | | | |
| Arachidonic | 9.48E−07 | 17/226 | |
| Acid Metab. | | | |
| Fatty Acid | 4.91E−06 | 16/192 | |
| Metab. | | | |
| LPS-IL- | 6.25E−05 | 18/205 | Apoc1 (36), Apoc2 (23), |
| Mediated | | | Cyp2c9 (32), Cyp3a4 (26) |
| Inhibition | | | |
| of RXR | | | |
| Function | | | |
| Tryptophan | 1.01E−04 | 15/255 | Cyp2c7 (103); Cyp4f12 (13); |
| Metabolism | | | Aadat (3); Cyp2a2 (15); |
| (“Metab.”) | | | Mettl7b (9) |

**These genes are differentially expressed in all 4 canonical pathways listed.

As shown in Table 7, transcriptional changes in several mRNA biomarkers in canonical pathways involving immune cell trafficking and inflammatory response were noted with both treatments. However, within this category, some differences were noted (i.e., differences in subcategories). The top canonical pathways that were the most significantly affected by APAP treatment were all involved either in the immune response (antigen presentation, receptor-mediated phagocytosis in macrophages and monocytes, lipid antigen presentation by CD1, or B-cell receptor signaling), extracellular interactions (integrin signaling—extracellular matrix effects; and ephrin receptor signaling—cell-to-cell communication), or membrane-related changes (virus entry via endocytic pathways, caveolar-mediated endocytosis). mRNA biomarkers with the greatest changes following APAP treatment readily fit in this group, with several cytokines, transporters and cell signaling molecules represented. Treatment-related changes in mRNA biomarkers associated with oxidative stress generation, apoptosis induction and necrosis (as based on corresponding protein function) were also evident and are consistent with APAP-induced hepatotoxicity. No increases in CYP450-related mRNAs were found.

The canonical pathways that were the most significantly affected by DGAL treatment were all involved in the immune response: acute phase response signaling, complement system and coagulation system changes. As with APAP, most of the mRNA biomarkers with the greatest increases following DGAL treatment fall into these categories, including mRNAs for various apolipoproteins, fibrinogens, and serine protease inhibitors. A canonical pathway greatly impacted was lipid metabolism, with molecular transport and small molecule biochemistry also scoring high, in large part due to lipid-involving pathways being altered (e.g., metabolism, transport, modification, etc.). These changes are consistent with previous reports of alterations in lipid metabolism and the composition of phospholipid membranes in DGAL-induced hepatotoxicity. Canonical pathways involved in various metabolic pathways, as exemplified by linoleic acid, arachidonic acid, fatty acid and CYP450-mediated metabolism, were significantly affected.

The mRNA biomarkers in the plasma result from multiple mechanisms of release (i.e., active processes and necrosis), multiple tissues, and even from different locations within an organ (e.g., centrilobular versus periportal injury). Pathway analysis for these two agents capable of causing liver perturbations, used as examples herein, revealed the majority of differentially expressed (as compared to respective reference values) mRNA biomarkers were related to hematological and immunological functions. Transcriptional alterations of genes related to hematological system function may be explained because the liver is a source of many of these proteins, and that release of mRNAs follows necrosis. In contrast, the mRNAs related to immunological functions may be derived from cells in the immune system responding to the necrotic damage within the liver.

The mechanistic interpretation of these gene expression profiles could be improved through the purification of liver-specific microparticles. Protein composition of microparticles has been demonstrated to be cell-type specific and proteomic studies in mouse hepatocytes, rat hepatocytes, and human haptocytes have identified liver-specific membrane proteins that could be used in antibody-based capture approaches to isolate microparticles to which are associated mRNA biomarkers released as a result of liver perturbation. Such membrane proteins may include, but are not limited to, HDL receptor protein, human liver-specific antigen1 (HLSA1), asialoglycoprotein receptor, liver-specific protein, and liver cell membrane antigen.

Antibodies to these liver-specific proteins and antibody-based capture techniques (e.g., affinity chromatography) are well known in the art. By enriching for liver-specific microparticles and performing gene expression microarray analysis on these particles, mechanistic interpretation of the profiles could be done without confounding effects of other tissues and release by necrosis. Such techniques might allow analysis of the liver transcriptome even in healthy individuals without liver injury. Thus, as an optional step in the method of the present invention, from a biological sample are isolated microparticles having associated therewith mRNA biomarkers, wherein the microparticles are isolated by an antibody-based capture technique via an antibody's binding affinity and specificity for a liver-specific membrane protein present on the surface of the microparticle. From the preparation of isolated microparticles, RNA comprising mRNA biomarkers is either isolated and then subjected to detection of mRNA biomarkers (via mRNA itself, or as cDNA or cDNA fragment following reverse transcription of the mRNA biomarker), or directly detected in the microparticle preparation.

In summary of Examples 1-5 herein, demonstrated is that circulating RNA comprising mRNA biomarkers can hold potential advantages over traditional biochemical-based (e.g., enzyme activity or protein level) biomarkers in assessing liver perturbations such as liver injury and toxicity. First, demonstrated is that mRNA biomarkers can show greater sensitivity and specificity for liver perturbations than traditional biochemical-based biomarkers.

Second, the two distinct agents used to illustrate the methods of the present invention, and known to induce liver perturbation comprising hepatotoxicity, each demonstrated distinct mRNA biomarkers (which could be used to generate a mRNA biomarker profile or panel of mRNA biomarkers comprising distinct mRNA biomarkers for one or more of that drug, or drug class for which that agent is a member) as compared to the other agent; and thus, circulating mRNA biomarkers may be useful in identifying the causative agent (e.g., one or more of drug or drug class) of liver injury or other liver perturbation (and a panel may be useful in such diagnosis; “diagnostic panel”). For example, see Table 6, “Circulating mRNA biomarkers Increased in APAP Experiment Only”, from which two or more mRNA biomarkers may be selected in creating an mRNA biomarker profile or diagnostic panel indicative of liver perturbation caused by an individual (single) drug such as acetaminophen. Using the method of the present invention, and detecting mRNA biomarkers indicative of liver perturbation from other members of the drug class of NSAIDs, one can compare those mRNA biomarkers detected, select two or more mRNA biomakers in common among the NSAIDs tested, and create a diagnostic panel or profile of mRNA biomakers indicative of liver perturbation from other members of NSAIDs. The same approach may be used to create mRNA biomarkers indicative of liver perturbation caused by other classes of drugs.

Finally, the findings that microparticles are actively released by hepatocytes, and that mRNA biomarkers are associated with the released nnicroparticles found in a biological sample, suggest that these nnicroparticles could provide a “virtual biopsy” of the transcriptional state of the liver that could be further exploited in assessing the liver response to perturbations of the liver in the absence of overt liver injury.

### Example 6

In this example, shown is a method for assessing the likelihood or for detecting the presence of liver injury or other liver perturbations in an individual suspected of having liver injury or other liver perturbations and with a history of taking a drug which has been causally-related or known to cause drug-induced liver injury. The suspected DILI causing drugs have been described previously herein, and include drugs in the drug classes selected from the group consisting of NSAIDs, antimicrobials, central nervous system agents, muscle relaxants, and antineoplastic agents. Performed was an assessment of circulating mRNA biomarkers in humans suffering from liver perturbation such as drug-induced liver injury (DILI). These individuals were enrolled in a study of DILI, and were patients admitted to a hospital for suspected DILI due to elevations in liver function tests.

Patient blood samples were collected in blood collection tubes containing EDTA, and the tubes were centrifuged at 1300×g for 15 minutes at room temperature. The supernatant was centrifuged again at 2,700×g for 15 minutes and the cell-free plasma supernatant was aliquotted and immediately stored at −80° C. Aliquots of the cell-free plasma were thawed and RNA was isolated using a commercially available RNA isolation kit following the manufacturer's instructions. Briefly, the cell-free plasma was added to buffer containing carrier linear acrylamide (10 μg), vortexed and allowed to incubate at room temperature for 10 minutes. After a brief centrifugation, 96% nondenatured ethanol was added to the sample and vortexed. After another brief centrifugation, the sample was added to a spin column, centrifuged and the filtrate discarded. More sample was added to the column and processed in this manner until all of the sample was applied. Wash buffer-1 from the kit was added to the spin column, centrifuged, and the filtrate discarded. Wash buffer-2 of the kit was applied in the same manner. The column was centrifuged at 20,000×g for 3, and then 1 minute to remove residual ethanol. After placing the spin column in a new tube, the RNA was eluted in RNAse-free water and stored immediately at −80 C.

To assess levels of RNA comprising mRNA biomarkers, total RNA was reverse transcribed using a commercially available RT kit. The resulting cDNA was amplified using the Taqman Universal PCR master mix and FAM-MGB probes and primers. Gene expression assays targeting albumin (Alb), fibrinogen beta chain (Fgb), and haptoglobin (Hp) were used (see Table 8 for gene expression assay information).

TABLE 8
Gene Expression Assay and cDNA Information for
qPCR Analyses

| mRNA | | | | |
|---|---|---|---|---|
| biomarker | | | | Open |
| detected | SEQ ID | NCBI | ABI | Biosytems |
| as cDNA | NO: | RefSeq. # | Assay ID# | Clone ID #* |
| Alb | 4 | NM_000477.5 | Hs00609411_m1 | 4734617 |
| Fgb | 5 | NM_005141.3 | Hs00905942_m1 | 4734415 |
| Hp | 6 | NM_005143.3 | Hs00605928_g1 | 4716454 |

*Open Biosystems cDNA was prepared and purified for use in standard curve generation.

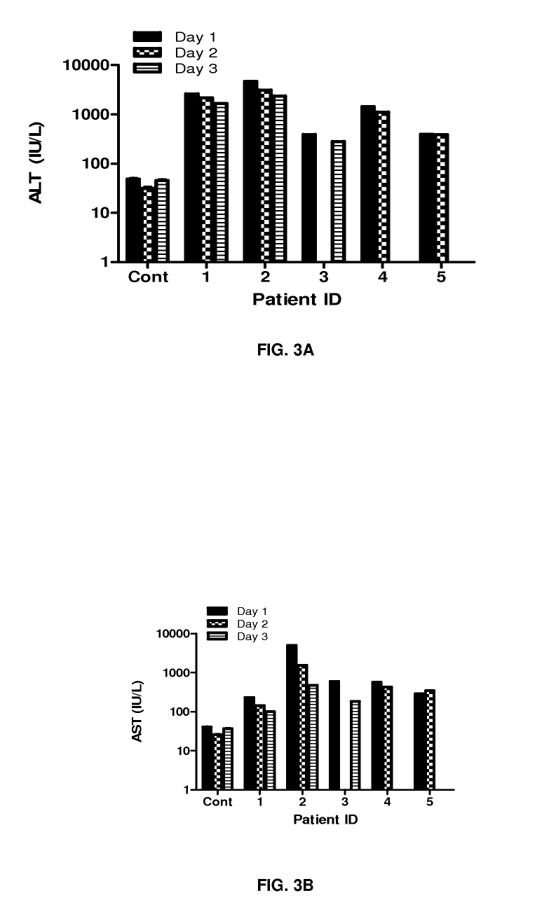

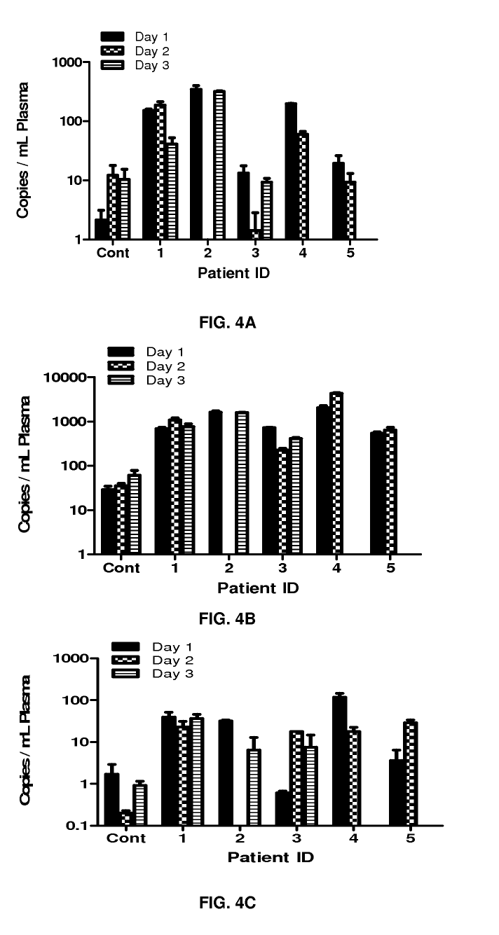

Data is presented for 5 patients, and the study control individuals (e.g., for reference values). FIGS. 3A and B show the ALT/AST serum enzyme values of patients and of the study control individuals exhibited during the study enrollment period. For patients 1, 2, & 5, acetaminophen is the confirmed or suspected causative agent of the observed DILI. In FIGS. 4A, B, and C, quantities of Alb mRNA biomarker (SEQ ID NO:4), Fgb mRNA biomarker (SEQ ID No:5), and Hp mRNA biomarker (SEQ ID NO:6), respectively, are presented as copy number per mL plasma from the patients and the study control individuals (reference values). From these results, it is clear that (a) induction of circulating (in body fluid) mRNA biomarker levels corresponds well with the induction of serum ALT/AST enzyme levels; and (b) the difference (fold increases) observed in the circulating mRNA biomarkers over the respective reference values are comparable to or greater than the differences (fold increases) of ALT/AST enzyme levels over the control values. Hence, these human mRNAs serve as mRNA biomarkers useful for detecting liver perturbation, such as DILI.

As demonstrated by Examples 1-6, the invention demonstrates elevated levels of RNA biomarkers comprising mRNA found in a biological sample in a rodent model of liver injury or liver enzyme induction and, in a correlative manner, elevated levels of RNA biomarkers comprising mRNA found in a biological sample of patients having liver perturbation. Further demonstrated is the utility of an animal model as a standard in vivo model for identifying RNA biomarkers comprising mRNA for detection of liver perturbation, such as liver injury or liver enzyme induction, in humans.

What is claimed is:

1. A method for detecting liver perturbation in an individual, the method comprising:
(a) detecting, in a biological sample obtained from the individual, a level of one or more RNA biomarkers comprising mRNA biomarkers;
(b) comparing the level of one or more mRNA biomarkers detected from step (a) with a reference value for each of the one or more RNA biomarkers detected from step (a);
whereby a difference in the level of the one or more mRNA biomarkers as compared to the reference value is indicative of liver perturbation.

2. The method of claim 1, wherein the biological sample is a body fluid comprising blood or a processed fraction thereof.

3. The method of claim 1, wherein the liver perturbation is caused by exposure to a drug.

4. The method of claim 3, wherein the drug is a drug in a drug class selected from the group consisting of NSAIDs, antimicrobials, central nervous system agents, muscle relaxants, and antineoplastic agents.

5. The method of claim 1, wherein the liver perturbation comprises one or more of:
liver injury; induction of one or more liver enzymes; and induction of a canonical pathway listed in Tables 6 and 7 herein.

6. The method of claim 1, wherein the mRNA biomarker is selected from the mRNA biomarkers listed in Table 6.

7. The method of claim 1, wherein microparticles, having associated therewith mRNA biomarkers, are first isolated from the biological sample prior to detecting a level of one or more RNA biomarkers comprising mRNA biomarkers.

8. The method of claim 1, wherein RNA biomarkers comprising mRNA are first reverse-transcribed to cDNA or a fragment thereof, and amplified, in detecting the level of one or more RNA biomarkers comprising mRNA.

9. A method of using the mRNA biomarkers detected by the method of claim 1 to generate a panel or profile of two or more of mRNA biomarkers indicative of liver perturbation from exposure to an individual drug or members of a drug class.


SEQUENCE LISTING (106 sequences)

SEQ ID NO: 1 - DNA, 2020 bp, Rattus norvegicus
  misc_feature (1)..(2020): cDNA for albumin
ctagcctctggcacaatgaagtgggtaacctttctcctcctcctcttcatctccggttctgccttttccaggggtgtgtttcgccgagaagcacacaagagtgagatcgcccatcggtttaaggacttgggagaacagcatttcaaaggcctagtcctgattgccttttcccagtatctccagaaatgcccatatgaagagcatatcaaattggtgcaggaagtaacagactttgcaaaaacatgtgtcgctgatgagaatgccgaaaactgtgacaagtccattcacactctcttcggagacaagttatgcgccattccaaagcttcgcgacaactacggtgaactggctgactgctgtgcaaaacaagagcccgaaagaaacgagtgtttcctgcagcacaaggatgacaaccccaacctgccacccttccagaggccggaggctgaggccatgtgcacctccttccaggagaaccctaccagctttctgggacactatttgcatgaagttgccaggagacatccttatttctatgccccagaactcctttactatgctgagaaatacaatgaggttctgacccagtgctgcacagagtctgacaaagcagcctgcctgacaccgaagcttgatgccgtgaaagagaaagcactggtcgcagctgtccgtcagaggatgaagtgctccagtatgcagagatttggagagagagccttcaaagcctgggcagtagctcgtatgagccagcgattccccaatgctgagttcgcagaaatcaccaaattggcaacagacctcaccaaaatcaacaaggagtgctgtcacggcgacctgttggaatgcgcggatgacagggcggaacttgccaagtacatgtgtgagaaccaggccactatctccagcaaactgcaggcttgctgtgataagccagtgctgcagaaatcccagtgtctcgctgagatagaacatgacaacattcctgccgatctgccctcaatagctgctgactttgttgaggataaggaagtgtgtaagaactatgctgaggccaaggatgtcttcctgggcacgtttttgtatgaatattcaagaaggcaccccgattactccgtgtccctgctgctgagacttgctaagaaatatgaagccacactggagaagtgctgtgctgaaggcgatcctcctgcctgctacggcacagtgcttgcagaatttcagcctcttgtagaagaacctaagaacttggtcaaaactaactgtgagctttacgagaagcttggagagtatggattccaaaacgccattctggttcgatacacccagaaagcacctcaggtgtcgaccccaactctcgtggaggcagcaagaaacctgggaagagtgggcaccaagtgttgtacccttcctgaagctcagagactgccctgtgtggaagactatctgtctgccatcctgaaccgtctgtgtgtgctgcatgagaagaccccagtgagcgagaaggtcaccaagtgctgtagtgggtccctggtggaaagacggccatgtttctctgctctgacagttgacgagacatatgtccccaaagagtttaaagctgagaccttcaccttccactctgatatctgcacactcccagacaaggagaagcagataaagaagcaaacggctctcgctgagctggtgaaacacaagcccaaggccacagaagatcagctgaagacggtgatgggtgacttcgcacaattcgtggacaagtgttgcaaggctgccgacaaggataactgcttcgccactgaggggccaaaccttgttgctagaagcaaagaagccttagcctaaacacatcacaaccatctcaggctaccctgagaaaaaaagacatgaagactcaggactcatctcttctgttggtgtaaaaccaacaccctaaggaacacaaatttctttgaacatttgacttcttttctctgtgccgcaattaataaaaaatagaaagaaaaaaaaaaaaaaaaaaaaa

SEQ ID NO: 2 - DNA, 1604 bp, Rattus norvegicus
  misc_feature (1)..(1604): cDNA for fibrinogen beta chain
ggcaaagggcaactgcttggaatttcagaaaatctgaaaccatgaggcatctatggctgctgctcctgtctgtttccttagttcaaactcaagctgcaactactgactccgataaagtggacctctcgattgcccgtggtcatcgacctgttgacaggagaaaggaagagcctcctagcctaagacctgcccctcctcctatcagcggaggtggctatcgagcccgtccagccaaagttgatgcaggtcagaagaaagtggaacgaaaaccccctgatgcaggaggctgtgttcacggtgacggagacatgggagtgctatgtcctacagggtgtgagttgcgtcaaactttgctaaaccacgaaaggccaatcaaaaacagtattgctgagttaaatagcaacataaactctgtttctgagacctcttcggtcacctttcagtacctgactctgctgaaagacatgtggaaaaagaagcaggcacaagttaaagataatgaaaatgtcataaatgagtattcctccattttggaagaccagaaattatatattgatgagactgtgaacgataatatcccccttaatcttcgtgtgctccgctcaatcctggaggacctgagaagcaaaatacaaaaattggagtctgacatctcagctcagacggaatactgccacactccatgcaccgtcaactgcaacatcccggtggtgtctgggaaagagtgtgaagagatcattcggaagggaggtgagacatccgaaatgtatctcattcagcctgacacctccagcaaaccatacagagtgtactgcgacatgaagaccgaaaacggaggatggacggtcatacagaaccgtcaggatggcagcgtcgactttggcaggaaatgggacccatacaaaaaaggatttggaaatattgcaaccaacgaagatacgaagaagtactgtggcttgccaggcgaatattggcttggcaatgataagattagccagctcaccaggataggacccacagaactcctaatcgaaatggaggactggaaaggagacaaggtgaaggcacactatggaggcttcacggtgcagaccgaggccaacaagtaccaagtgtcagtgaacaagtacaaagggactgctggcaacgccctcatggaaggggcctctcagctggtgggggagaacagaaccatgaccatccacaacggcatgttcttcagcacctacgacagggacaacgatggctgggtaactacagatccaagaaagcagtgctcaaaagaagatggcggcggctggtggtataacagatgccatgcagccaatccgaatggcagatactactggggtggcctttacagctgggacatgtccaagcacggcacagatgatggtgtggtgtggatgaactggaagggatcgtggtactccatgagaaggatgtctatgaagatcaggcccgtcttcccacagcaatagtcccccaagcatagactttcagccttctatgtctgacattttttacattatgttattggaattttctttgatacattatatccactaatgctcaaaaaaaaaaaaaaaaaaaaaaaaaaaaaa

SEQ ID NO: 3 - DNA, 1239 bp, Rattus norvegicus
  misc_feature (1)..(1239): cDNA for haptoglobin
agagaggcccacgatgagagccctgggagctgtcgtcactctcctgctctggggtcagctttttgctgtggaattgggcaatgatgccacagacattgaagatgacagctgcccaaagcccccagagattgcaaacggctatgtggaacacttggttcgttatcgctgccgacagttctacaaactacagaccgaaggagatggaatctacaccttaaacagtgagaagcaatgggtgaacccagctgctggcgataaactccccaagtgtgaggcagtgtgtgggaagcccaagcatcctgtggaccaggtacagcgcatcatcggtggttccatggacgccaaaggcagctttccttggcaggccaagatgatctccagacatggactcaccactggggccacactgatcagtgaccagtggctgctgaccactgcccaaaacctcttcctgaatcacagtgagaatgcgacagccaaggacattgcccctaccttaacactctatgtggggaaaaaccagctggtggagattgagaaggtagttctccaccccgagcgctctgtggtggatatcgggctgatcaagctcaaacagaaagtgcttgtcactgagaaagtcatgcctatctgcctgccttccaaagactacgtagcgccaggccgcatgggctatgtgtccggttgggggcggaatgtcaactttagatttactgaacgtctcaagtatgtcatgctgcctgtggctgaccaggagaagtgtgagctgcactatgagaaaagcacagtgcctgagaagaaaggcgctgtaagtcctgttggggtacagcccatcttgaataagcataccttctgtgctggccttaccaagtatgaggaagacacttgctatggtgacgctggcagtgcctttgccgtccatgacacggaggaggacacctggtatgcagctgggatcctgagctttgacaagagttgtgccgtagctgagtatggtgtgtacgtgagggcaactgatctgaaggactgggtccaggaaacaatggccaagaactagttcagggctgactagagggctgcacacagtggggcagggcaattcaccctggaagaggaagtagaagggttggggacataatctgagggctgctagccctgcattgctcagtcaataataaaaaacgagctttggacccaaaaaaaaaaaaaaaaaaaaaaaaaaaaaaaaaaaaaaaaaaa

SEQ ID NO: 4 - DNA, 2264 bp, Homo sapiens
  misc_feature (1)..(2264): cDNA for albumin
agtatattagtgctaatttccctccgtttgtcctagcttttctcttctgtcaaccccacacgcctttggcacaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataacatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaaaaaaaaaaaaaaaaaaa

SEQ ID NO: 5 - DNA, 1963 bp, Homo sapiens
  misc_feature (1)..(1963): cDNA for fibrinogen beta
agatatatataggattgaagatctctcagttaagtctacatgaaaaggatggtttcttggagcttccacaaacttaaaaccatgaaacatctattattgctactattgtgtgtttttctagttaagtcccaaggtgtcaacgacaatgaggagggtttcttcagtgcccgtggtcatcgaccccttgacaagaagagagaagaggctcccagcctgaggcctgccccaccgcccatcagtggaggtggctatcgggctcgtccagccaaagcagctgccactcaaaagaaagtagaaagaaaagcccctgatgctggaggctgtcttcacgctgacccagacctgggggtgttgtgtcctacaggatgtcagttgcaagaggctttgctacaacaggaaaggccaatcagaaatagtgttgatgagttaaataacaatgtggaagctgtttcccagacctcctcttcttcctttcagtacatgtatttgctgaaagacctgtggcaaaagaggcagaagcaagtaaaagataatgaaaatgtagtcaatgagtactcctcagaactggaaaagcaccaattatatatagatgagactgtgaatagcaatatcccaactaaccttcgtgtgcttcgttcaatcctggaaaacctgagaagcaaaatacaaaagttagaatctgatgtctcagctcaaatggaatattgtcgcaccccatgcactgtcagttgcaatattcctgtggtgtctggcaaagaatgtgaggaaattatcaggaaaggaggtgaaacatctgaaatgtatctcattcaacctgacagttctgtcaaaccgtatagagtatactgtgacatgaatacagaaaatggaggatggacagtgattcagaaccgtcaagacggtagtgttgactttggcaggaaatgggatccatataaacagggatttggaaatgttgcaaccaacacagatgggaagaattactgtggcctaccaggtgaatattggcttggaaatgataaaattagccagcttaccaggatgggacccacagaacttttgatagaaatggaggactggaaaggagacaaagtaaaggctcactatggaggattcactgtacagaatgaagccaacaaataccagatctcagtgaacaaatacagaggaacagccggtaatgccctcatggatggagcatctcagctgatgggagaaaacaggaccatgaccattcacaacggcatgttcttcagcacgtatgacagagacaatgacggctggttaacatcagatcccagaaaacagtgttctaaagaagacggtggtggatggtggtataatagatgtcatgcagccaatccaaacggcagatactactggggtggacagtacacctgggacatggcaaagcatggcacagatgatggtgtagtatggatgaattggaaggggtcatggtactcaatgaggaagatgagtatgaagatcaggcccttcttcccacagcaatagtccccaatacgtagatttttgctcttctgtatgtgacaacatttttgtacattatgttattggaattttctttcatacattatattcctctaaaactctcaagcagacgtgagtgtgactttttgaaaaaagtataggataaattacattaaaatagcacatgattttcttttgttttcttcatttctcttgctcaccaagaagtaacaaaagtatagttttgacagagttggtgttcataatttcagttctagttgattgcgagaattttcaaataaggaagaggggtcttttatccttgtcgtaggaaaaccatgacggaaaggaaaaactgatgtttaaaagtccacttttaaaactatatttatttatgtaggatctgtcaaagaaaacttccaaaaagatttattaattaaaccagactctgttgcaataaaaaaaaaaaaaa

SEQ ID NO: 6 - DNA, 1461 bp, Homo sapiens
  misc_feature (1)..(1461): cDNA for haptoglobin
agatgccccacagcactgctcttccagaggcaagaccaaccaagatgagtgccctgggagctgtcattgccctcctgctctggggacagctttttgcagtggactcaggcaatgatgtcacggatatcgcagatgacggctgcccgaagccccccgagattgcacatggctatgtggagcactcggttcgctaccagtgtaagaactactacaaactgcgcacagaaggagatggagtatacaccttaaatgataagaagcagtggataaataaggctgttggagataaacttcctgaatgtgaagcagatgacggctgcccgaagccccccgagattgcacatggctatgtggagcactcggttcgctaccagtgtaagaactactacaaactgcgcacagaaggagatggagtgtacaccttaaacaatgagaagcagtggataaataaggctgttggagataaacttcctgaatgtgaagcagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccataaaaaaaaaaaaaaaaaaaaaaaaaaaaaa

SEQ ID NO: 7 - DNA, 780 bp, Rattus norvegicus
  misc_feature (1)..(780): cDNA for chemokine ligand 2
gcagagacacagacagaggccagcccagaaaccagccaactctcactgaagccagatctctcttcctccaccactatgcaggtctctgtcacgcttctgggcctgttgttcacagttgctgcctgtagcatccacgtgctgtctcagccagatgcagttaatgccccactcacctgctgctactcattcactggcaagatgatcccaatgagtcggctggagaactacaagagaatcaccagcagcaggtgtcccaaagaagctgtagtatttgtcaccaagctcaagagagagatctgtgctgaccccaataaggaatgggtccagaagtacattagaaaactggaccagaaccaagtgagatcagaaactacagtcttctataaaattgcatcaaccctaaggacttcagcacctttgaatgtgaacttgacccataaatctgaagctaatgcatccactctcttttccacaaccacctcaagcacttctgtagaagtgaccagtatgacagagaactagtgtgatttggaatgtgatgccttaagtaatgttaaacttatttaacttattgatattacactattcccttccatgaatactagaaatccttaaatgcaagatgtagatccatttttttatttctctgtgaatcctggttcaacactttcaatgtatgagagatgaatgggtaaactttgtgtttgagagtccaaggtattgtttaaaatattattatggatattcctaattattaaaagaaatatattatttttgtac

SEQ ID NO: 8 - DNA, 760 bp, Homo sapiens
  misc_feature (1)..(760): cDNA for chemokine ligand 2
gaggaaccgagaggctgagactaacccagaaacatccaattctcaaactgaagctcgcactctcgcctccagcatgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttgaacactcactccacaacccaagaatctgcagctaacttattttcccctagctttccccagacaccctgttttattttattataatgaattttgtttgttgatgtgaaacattatgccttaagtaatgttaattcttatttaagttattgatgttttaagtttatctttcatggtactagtgttttttagatacagagacttggggaaattgcttttcctcttgaaccacagttctacccctgggatgttttgagggtctttgcaagaatcattaatacaaagaattttttttaacattccaatgcattgctaaaatattattgtggaaatgaatattttgtaactattacaccaaataaatatatttttgtacaaaaaaaaaaaaaaa

SEQ ID NO: 9 - DNA, 1603 bp, Rattus norvegicus
  misc_feature (1)..(1603): cDNA for serpin peptidase inhibitor
tggctccagacaaaatatagacgcaccaccagcctggctaggaagtaatgcttcccatgagcccccaaagaacgggaaaccagacactgcaggcaaacatctccttcttggggagtctggaagccagatatccataccaccaatgctgtgatgtcagccatgtctcccggcaacctgttgcagggtggtgctcacccatggcacaagaggaaacagatggcaaacattcctggaaactgaattgccaaggaaaatggcaccctccatctcacgggggctcctgcttctggcaggcctgtgttgcctggcccccagcttcctggctgaggatgcccaggaaaccgatacctcccagcaggaccagagtccaacctaccgtaagatttcttcaaacctggcagactttgccttcagcctataccgggagctggtccatcaatccaatacatccaacatcttcttctcccctatgagcatcaccacagccttcgccatgctctccctggggagcaagggtgacactcgcaaacagattctagagggcctggagttcaacctcacacagatacctgaggctgacatccacaaggccttccatcacctcctccaaactctcaacaggccagacagtgagctgcagctgaacacaggcaatggcctctttgtcaacaagaatctgaagctggtggagaagtttctggaagaggtcaagaacaattaccactcagaagccttctctgtcaactttgccgactcagaagaggctaagaaagtaattaatgattatgtagagaagggaacccaaggaaagatagttgatttgatgaaacagctggacgaagacacggtttttgccctggtgaattacattttctttaaaggcaagtggaagaggccattcaatcctgagcacactagggatgctgacttccacgtagacaagtccaccacggtgaaggtgcccatgatgaaccgcctgggcatgtttgacatgcactattgcagcacactgtccagctgggtgctgatgatggattacctgggcaacgccactgccatcttcctcctgcccgatgatggcaagatgcagcatctggagcaaactctcaccaaggatctcatttcccggttcctgctaaacaggcaaacaaggtcagccattctctacttccccaaactgtccatctctggaacctataacttgaagacactcctgagctcactgggcatcacccgggtcttcaacaatgatgctgatctctctggaatcacagaggatgcccccctgaagcttagccaggctgtgcataaggctgtgctgaccttagatgagaggggaacagaggctgcaggagccactgtggtggaggccgtccccatgtctctgccccctcaagtgaagttcgaccaccctttcattttcatgatagttgaatcagaaactcagagccccctctttgtgggaaaagtgatagatcccacacgttaatcactgtcctcagaagtcacatcccttctggatcgggtccccttcctaataatattaaactcaggctggcctggcctgatgcttctgcaaaaaaaaaaaaaaaaaaaaaaaaaa

SEQ ID NO: 10 - DNA, 3220 bp, Homo sapiens
  misc_feature (1)..(3220): cDNA for serpin peptidase inhibitor
acaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcatgtgactgtaaatccctcccatgttttctctgagtctccctttgcctgctgaggctgtatgtgggctccaggtaacagtgctgtcttcgggccccctgaactgtgttcatggagcatctggctgggtaggcacatgctgggcttgaatccaggggggactgaatcctcagcttacggacctgggcccatctgtttctggagggctccagtcttccttgtcctgtcttggagtccccaagaaggaatcacaggggaggaaccagataccagccatgaccccaggctccaccaagcatcttcatgtccccctgctcatcccccactcccccccacccagagttgctcatcctgccagggctggctgtgcccaccccaaggctgccctcctgggggccccagaactgcctgatcgtgccgtggcccagttttgtggcatctgcagcaacacaagagagaggacaatgtcctcctcttgacccgctgtcacctaaccagactcgggccctgcacctctcaggcacttctggaaaatgactgaggcagattcttcctgaagcccattctccatggggcaacaaggacacctattctgtccttgtccttccatcgctgccccagaaagcctcacatatctccgtttagaatcaggtcccttctccccagatgaagaggagggtctctgctttgttttctctatctcctcctcagacttgaccaggcccagcaggccccagaagaccattaccctatatcccttctcctccctagtcacatggccataggcctgctgatggctcaggaaggccattgcaaggactcctcagctatgggagaggaagcacatcacccattgacccccgcaacccctccctttcctcctctgagtcccgactggggccacatgcagcctgacttctttgtgcctgttgctgtccctgcagtcttcagagggccaccgcagctccagtgccacggcaggaggctgttcctgaatagcccctgtggtaagggccaggagagtccttccatcctccaaggccctgctaaaggacacagcagccaggaagtcccctgggcccctagctgaaggacagcctgctccctccgtctctaccaggaatggccttgtcctatggaaggcactgccccatcccaaactaatctaggaatcactgtctaaccactcactgtcatgaatgtgtacttaaaggatgaggttgagtcataccaaatagtgatttcgatagttcaaaatggtgaaattagcaattctacatgattcagtctaatcaatggataccgactgtttcccacacaagtctcctgttctcttaagcttactcactgacagcctttcactctccacaaatacattaaagatatggccatcaccaagccccctaggatgacaccagacctgagagtctgaagacctggatccaagttctgacttttccccctgacagctgtgtgaccttcgtgaagtcgccaaacctctctgagccccagtcattgctagtaagacctgcctttgagttggtatgatgttcaagttagataacaaaatgtttatacccattagaacagagaataaatagaactacatttcttgca

SEQ ID NO: 11 - DNA, 904 bp, Rattus norvegicus
  misc_feature (1)..(904): cDNA for retinol binding protein 4
gcggcggccaggcttgcacgcggcttctgctgggcagactccggtgtgaaatggagtgggtgtgggcgctcgtgctgctggcggctctgggaggcggcagcgccgagcgcgactgcagggtgagcagcttcagagtcaaggagaacttcgacaaggctcgtttctctgggctctggtatgccatcgccaaaaaggatcccgagggtctctttttgcaagacaacatcatcgctgagttttctgtggacgagaagggtcatatgagcgctacagccaagggacgagtccgtcttctgagcaactgggaagtgtgtgcagacatggtgggcactttcacagacacagaagatcctgccaagttcaagatgaagtactggggtgtagcctcctttctccagcgaggaaacgatgaccactggatcatcgatacggactacgacaccttcgctctgcagtattcctgccgcctgcagaatctggatggcacctgtgcagacagctactcctttgtgttttctcgtgaccccaatggcctgaccccggagacacggaggctggtgaggcagcgacaggaggagctgtgcctagagaggcagtacagatggatcgagcacaatggttactgtcaaagcagaccctcaagaaacagtttgtagcaacgtcaaggatgtataaagttggaaaacttctgattagctctcatccagtcttcatctctatttatcttagaagtttagtttccccacctcccctcccttctctaggtggacattaaaaccatcgtccaaatacatgggaatgcctgaatccattcacacaaacgtgtatcttactgagaagttccccgagagacgtttgaggcttgggattccaaacttgatttattaaacgtatagtcaccatc

SEQ ID NO: 12 - DNA, 941 bp, Homo sapiens
  misc_feature (1)..(941): cDNA for retinol binding protein 4, plasma
cgcctccctcgctccacgcgcgcccggactcggcggccaggcttgcgcgcggttcccctcccggtgggcggattcctgggcaagatgaagtgggtgtgggcgctcttgctgttggcggcgctgggcagcggccgcgcggagcgcgactgccgagtgagcagcttccgagtcaaggagaacttcgacaaggctcgcttctctgggacctggtacgccatggccaagaaggaccccgagggcctctttctgcaggacaacatcgtcgcggagttctccgtggacgagaccggccagatgagcgccacagccaagggccgagtccgtcttttgaataactgggacgtgtgcgcagacatggtgggcaccttcacagacaccgaggaccctgccaagttcaagatgaagtactggggcgtagcctcctttctccagaaaggaaatgatgaccactggatcgtcgacacagactacgacacgtatgccgtgcagtactcctgccgcctcctgaacctcgatggcacctgtgctgacagctactccttcgtgttttcccgggaccccaacggcctgcccccagaagcgcagaagattgtaaggcagcggcaggaggagctgtgcctggccaggcagtacaggctgatcgtccacaacggttactgcgatggcagatcagaaagaaaccttttgtagcaatatcaagaatctagtttcatctgagaacttctgattagctctcagtcttcagctctatttatcttaggagtttaatttgcccttctctccccatcttccctcagttcccataaaaccttcattacacataaagatacacgtgggggtcagtgaatctgcttgcctttcctgaaagtttctggggcttaagattccagactctgattcattaaactatagtcacccgtgtcctgtgaaaaaaaaaaaaa

SEQ ID NO: 13 - DNA, 663 bp, Rattus norvegicus
  misc_feature (1)..(663): cDNA for ubiquitin-like modifier
acatccacagctcagactttaagagagctgacccagtgagtctctctctgactgtgagagccagcagccagaagctgagagaagcagagagaagcagactccttagtttcaggggacctagtgctaaagcctacagccatgacctggaacctaaaggtgaagatgctggggggcaaggagttcctggtgtccatgacaaactccatgatgctgtcagaactgaagaagcaggttgctcagaagagtggtgtgccagctttccagcagcgcctggcccaccaaagcggtgaaatgctacaggatggtgttgcccttatcaggcagggccttagttctggcagcacagtcatgctgatggtagagaactgcagccatcctctgagcatcctggtgaggaatgaaaggggccgcagcaatgtctatgaggtccagctgacacagactgtggaagtgcttatgaggcaggtgtcccagcacgagcaagtctcccaagaccaattctggctgagcttcaacggaaggcccatggaggacaaagagccactgggggagtatggcctaacaccccactgcacagtgatcatgaatttgcgcctgagggggggaggggactactatttctaaggcccctatccagagccaatgtaggtaataaaagacaat

SEQ ID NO: 14 - DNA, 685 bp, Homo sapiens
  misc_feature (1)..(685): cDNA for ubiquitin-like modifier
ataatagggccggtgctgcctgccgaagccggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaaaaaaaaaaaaaaaaaaaa

SEQ ID NO: 15 - DNA, 387 bp, Rattus norvegicus
  misc_feature (1)..(387): cDNA for Metallothionein-2a
ctagaactctacagcgatctctcgttgatctccaactgccgcctccattcgccatggaccccaactgctcctgtgccacagatggatcctgctcctgcgctggctcctgcaaatgcaaacaatgcaaatgcacctcctgcaagaaaagctgctgttcctgctgccccgtgggctgtgcgaagtgctcccagggctgcatctgcaaagaggcttcggacaagtgcagctgctgcgcctgaagtgggggcgtcctcacaatggtgtaaataaaacaacgtaggaacctagcctttttttgtacaaccctgaccggttctccacacttttttctataaagcatgtaactgacaataaaataaaaaaacttgacttgattaacccagcttt

SEQ ID NO: 16 - DNA, 466 bp, Homo sapiens
  misc_feature (1)..(466): cDNA for metallothionein 2A
cttgccgcgctgcactccaccacgcctcctccaagtcccagcgaacccgcgtgcaacctgtcccgactctagccgcctcttcagctcgccatggatcccaactgctcctgcgccgccggtgactcctgcacctgcgccggctcctgcaaatgcaaagagtgcaaatgcacctcctgcaagaaaagctgctgctcctgctgccctgtgggctgtgccaagtgtgcccagggctgcatctgcaaaggggcgtcggacaagtgcagctgctgcgcctgatgctgggacagccccgctcccagatgtaaagaacgcgacttccacaaacctggattttttatgtacaaccctgaccgtgaccgtttgctatattcctttttctatgaaataatgtgaatgataataaaacagctttgacttgaaaaaaaaaaaaaaaaaaaaaaaaaaaaaaaaaaaaaaaaaaaaaaaa

SEQ ID NO: 17 - DNA, 2083 bp, Rattus norvegicus
  misc_feature (1)..(2083): cDNA for leukotriene A4 hydrolase
cctctatgcttccctcctttctcaggcttgcggtgaaagtctggcggtagagtgtctgggtgctggttgacgtgcggcggatctcccacaaccatgcctgaggtcgcggatacttgttccttggcttctccagcctcggtctgccgaacccagcacctccatcttcgctgcagcgtcgacttcgctcgccgggcactgaccgggaccgccgcgctcacggtccagtcacaggaggataatctgcgcagcctgactttggatacaaaagaccttacgatagaaaaagtggtgatcaacggacaagaagtcaaatacactcttggagaaagccagggttacaaaggatcgccgatggaaatctcccttcccatcgctctgagcaaaaatcaagaggtcgttatagagatttccttcgagacatctcccaagtcctcggcccttcagtggctcactcccgagcagacttcaggaaagcagcatccatacctcttcagtcagtgccaggccatccactgcagggcaatcctcccttgccaagacactccttctgtgaagttaacctacaccgcagaggtatctgtccccaaagaactggtggctctcatgagtgccatccgtgatggagaagcccctgacccagaagaccccagcaggaagatatacagatttaaccagagggttcccataccgtgctacctgattgctttagtcgttggcgctttagaaagcaggcaaattggcccaagaactctggtgtggtctgagaaagagcaggtggagaagtctgcttatgaattctctgagactgaatccatgcttaaaattgcagaagatctggggggaccctatgtttggggacagtacgacctgttggtcctgcccccatccttcccgtacggaggcatggagaatccttgtctcacattcgtaacccctactctattggcaggtgacaagtcactctctaatgttattgcacacgaaatatcgcatagctggacagggaacctagtgactaacaagacttgggatcacttctggttaaacgaaggacataccgtctatttagagcgccacatttgtgggcggttgtttggggaaaaattcagacatttccacgctttaggaggatggggagagctacagaacacgataaagactttcggggagtcccatcccttcaccaagctcgtggtcgatctgaaggacgtagaccccgatgtggcctactcctccattccctacgagaagggcttcgccttgctcttctacctggaacaacttcttgggggaccggaggtgtttctaggattcctaaaggcttatgtggagaagttttcctaccagagcgtaaccactgatgactggaagagtttcctgtatgctcactttaaagataaggtggatcttctcaatcaagttgattggaatgcctggctctatgctcctggcctgcctcctgtcaaacccaattacgatgtgactctcacgaatgcctgcatcgccttaagtcaaagatgggtcactgccaaagaggaagatttaaattcattcagcatcgaagacctgaaggacctctcgtcccatcagctgaatgagttcctggcacaggtgcttcagaaggcacctcttcccttggggcacataaagcgaatgcaagaggtgtacaacttcaatgccattaacaattctgaaatacgattcagatggttacggctctgcattcaatcgaagtgggaagaagcaattcctttggccctaaagatggcaactgaacaaggaaggatgaaatttacacgacctctattcaaggacctcgctgccttcgacaaatcccacgatcaagctgtccgcacctaccaggaacataaagcctgtatgcatcccgtgaccgccatgctggtggggaaagatctgaaagtggattaaggaactgagtgttgctgatttcagaaaattccttttttttaagaaaaaaaatcgaacctattaagaaatacaaaattttagctctcgttgtaaaaaaaaaaaaaaaaaaaaaaaaaaaaaaaaaaaaaaaaaaaaaaaaaaaaaaaaaaaaaaa

SEQ ID NO: 18 - DNA, 2060 bp, Homo sapiens
  misc_feature (1)..(2060): cDNA for leukotriene A4 hydrolase
ctctatcgacgagtctggtagctgagcgttgggctgtaggtcgctgtgctgtgtgatcccccagagccatgcccgagatagtggatacctgttcgttggcctctccggcttccgtctgccggaccaagcacctgcacctgcgctgcagcgtcgactttactcgccggacgctgaccgggactgctgctctcacggtccagtctcaggaggacaatctgcgcagcctggttttggatacaaaggaccttacaatagaaaaagtagtgatcaatggacaagaagtcaaatatgctcttggagaaagacaaagttacaagggatcgccaatggaaatctctcttcctatcgctttgagcaaaaatcaagaaattgttatagaaatttcttttgagacctctccaaaatcttctgctctccagtggctcactcctgaacagacttctgggaaggaacacccatatctctttagtcagtgccaggccatccactgcagagcaatccttccttgtcaggacactccttctgtgaaattaacctatactgcagaggtgtctgtccctaaagaactggtggcacttatgagtgctattcgtgatggagaaacacctgacccagaagacccaagcaggaaaatatacaaattcatccaaaaagttccaataccctgctacctgattgctttagttgttggagctttagaaagcaggcaaattggcccaagaactttggtgtggtctgagaaagagcaggtggaaaagtctgcttatgagttttctgagactgaatctatgcttaaaatagcagaagatctgggaggaccgtatgtatggggacagtatgacctattggtcctgccaccatccttcccttatggtggcatggagaatccttgccttacttttgtaactcctactctactggcaggcgacaagtcactctccaatgtcattgcacatgaaatatctcatagctggacagggaatctagtgaccaacaaaacttgggatcacttttggttaaatgagggacatactgtgtacttggaacgccacatttgcggacgattgtttggtgaaaagttcagacattttaatgctctgggaggatggggagaactacagaattcggtaaagacatttggggagacacatcctttcaccaaacttgtggttgatctgacagatatagaccctgatgtagcttattcttcagttccctatgagaagggctttgctttacttttttaccttgaacaactgcttggaggaccagagattttcctaggattcttaaaagcttatgttgagaagttttcctataagagcataactactgatgactggaaggatttcctgtattcctattttaaagataaggttgatgttctcaatcaagttgattggaatgcctggctctactctcctggactgcctcccataaagcccaattatgatatgactctgacaaatgcttgtattgccttaagtcaaagatggattactgccaaagaagatgatttaaattcattcaatgccacagacctgaaggatctctcttctcatcaattgaatgagtttttagcacagacgctccagagggcacctcttccattggggcacataaagcgaatgcaagaggtgtacaacttcaatgccattaacaattctgaaatacgattcagatggctgcggctctgcattcaatccaagtgggaggacgcaattcctttggcgctaaagatggcaactgaacaaggaagaatgaagtttacccggcccttattcaaggatcttgctgcctttgacaaatcccatgatcaagctgtccgaacctaccaagagcacaaagcaagcatgcatcccgtgactgcaatgctggtggggaaagacttaaaagtggattaaagacctgcgtattgatgattttagagatttctcttttttaaatggaattcgtaaagaaatataaaacttcagctcacaattaaaactgtctttttagttttggctttttattgttttgttggtgattttactgaaataaagatgagctacttcttc

SEQ ID NO: 19 - DNA, 2320 bp, Rattus norvegicus
  misc_feature (1)..(2320): cDNA for glycoprotein nmb
gtatttcataaaacagagaggatcgcaggaggccggcactctgactcctggtggatgggactagggagtcagagtcaagccctgactggctgagggcgggcgctccgagtcagcatggaaagtctctgcggggtcctggtatttctgctgctggctgcaggactgccgctccaggcggccaagcggttccgtgatgtgctgggccatgagcagtatccggatcacatgagggagaacaaccaattacgtggctggtcttcagatgaaaatgaatgggatgaacagctgtatccagtgtggaggaggggagagggcagatggaaggactcctgggaaggaggccgtgtgcaggcagccctaaccagtgattcaccggccttggtgggttccaatatcaccttcgtagtgaacctggtgttccccagatgccagaaggaagatgccaacggcaatatcgtctatgagaggaactgcagaagtgatttggagctggcttctgacccgtatgtctacaactggaccacaggggcagacgatgaggactgggaagacaacaccagccaaggccagcacctcaggttccccgacgggaagcccttccctcgcccccacggacggaagaaatggaacttcgtctacgtcttccacacacttggtcagtattttcaaaagctgggtcagtgttcagcacgagtttctataaacacagtcaacttgacagttggccctcaggtcatggaagtgattgtctttcgaagacacggccgggcatacattcccatctccaaagtgaaagacgtgtatgtgataacagatcagatccctatattcgtgaccatgtaccagaagaatgaccggaactcgtctgatgaaaccttcctcagagacctccccattttcttcgatgtcctcattcacgatcccagtcatttcctcaactactctgccatttcctacaagtggaactttggggacaacactggcctgtttgtctccaacaatcacactttgaatcacacgtatgtgctcaatggaaccttcaactttaacctcaccgtgcaaactgcagtgccgggaccatgcccctcacccacaccttcgccttcttcttcgacttctccttcgcctgcatcttcgccttcacccacattatcaacacctagtccctctttaatgcctactggctacaaatccatggagctgagtgacatttccaatgaaaactgccgaataaacagatatggttacttcagagccaccatcacaattgtagatggaatcctagaagtcaacatcatccaggtagcagatgtcccaatccccacactgcagcctgacaactcactgatggacttcattgtgacctgcaaaggggccactcccacggaagcctgtacgatcatctctgaccccacctgccagatcgcccagaacagggtgtgcagcccggtggctgtggatgagctgtgcctcctgtccgtgaggagagccttcaatgggtccggcacgtactgtgtgaatttcactctgggagacgatgcaagcctggccctcaccagcgccctgatctctatccctggcaaagacctaggctcccctctgagaacagtgaatggtgtcctgatctccattggctgcctggccatgtttgtcaccatggttaccatcttgctgtacaaaaaacacaagacgtacaagccaataggaaactgcaccaggaacgtggtcaagggcaaaggcctgagtgtttttctcagccatgcaaaagccccgttctcccgaggagaccgggagaaggatccactgctccaggacaagccatggatgctctaagtcttcactctcacttctgactgggaacccactcttctgtgcatgtatgtgagctgtgcagaagtacatgactggtagctgttgttttctacggattattgtaaaatgtatatcatggtttagggagtgtagttaattggcattttagtgaagggatgggaagacagtatttcttcgcatctgtattgtggtttttatactgttaatagggtgggcacattgtgtctgaagggggagggggaggtcactgctacttaaggtcctaggttaactgggagaggatgccccaggctccttagatttctacacaagatgtgcctgaacccagctagtcctgacctaaaggccatgcttcatcaactctatctcagctcattgaacatacctgagcgcctgatggaattataatggaaccaagcttgttgtatggtgtgtgtgtgtacataagatactcattaaaaagacagtctattaaaaaaaaaaaaaa

SEQ ID NO: 20 - DNA, 2775 bp, Homo sapiens
  misc_feature (1)..(2775): cDNA for glycoprotein  nmb
tgccgcttaataccatcacatgatcctccccgaggccctgtatttaattaaaatagagagggaggcaccacagatgccagaagaacactgttgctcttggtggacgggcccagaggaattcagagttaaaccttgagtgcctgcgtccgtgagaattcagcatggaatgtctctactatttcctgggatttctgctcctggctgcaagattgccacttgatgccgccaaacgatttcatgatgtgctgggcaatgaaagaccttctgcttacatgagggagcacaatcaattaaatggctggtcttctgatgaaaatgactggaatgaaaaactctacccagtgtggaagcggggagacatgaggtggaaaaactcctggaagggaggccgtgtgcaggcggtcctgaccagtgactcaccagccctcgtgggctcaaatataacatttgcggtgaacctgatattccctagatgccaaaaggaagatgccaatggcaacatagtctatgagaagaactgcagaaatgaggctggtttatctgctgatccgtatgtttacaactggacagcatggtcagaggacagtgacggggaaaatggcaccggccaaagccatcataacgtcttccctgatgggaaaccttttcctcaccaccccggatggagaagatggaatttcatctacgtcttccacacacttggtcagtatttccagaaattgggacgatgttcagtgagagtttctgtgaacacagccaatgtgacacttgggcctcaactcatggaagtgactgtctacagaagacatggacgggcatatgttcccatcgcacaagtgaaagatgtgtacgtggtaacagatcagattcctgtgtttgtgactatgttccagaagaacgatcgaaattcatccgacgaaaccttcctcaaagatctccccattatgtttgatgtcctgattcatgatcctagccacttcctcaattattctaccattaactacaagtggagcttcggggataatactggcctgtttgtttccaccaatcatactgtgaatcacacgtatgtgctcaatggaaccttcagccttaacctcactgtgaaagctgcagcaccaggaccttgtccgccaccgccaccaccacccagaccttcaaaacccaccccttctttagcaactactctaaaatcttatgattcaaacaccccaggacctgctggtgacaaccccctggagctgagtaggattcctgatgaaaactgccagattaacagatatggccactttcaagccaccatcacaattgtagagggaatcttagaggttaacatcatccagatgacagacgtcctgatgccggtgccatggcctgaaagctccctaatagactttgtcgtgacctgccaagggagcattcccacggaggtctgtaccatcatttctgaccccacctgcgagatcacccagaacacagtctgcagccctgtggatgtggatgagatgtgtctgctgactgtgagacgaaccttcaatgggtctgggacgtactgtgtgaacctcaccctgggggatgacacaagcctggctctcacgagcaccctgatttctgttcctgacagagacccagcctcgcctttaaggatggcaaacagtgccctgatctccgttggctgcttggccatatttgtcactgtgatctccctcttggtgtacaaaaaacacaaggaatacaacccaatagaaaatagtcctgggaatgtggtcagaagcaaaggcctgagtgtctttctcaaccgtgcaaaagccgtgttcttcccgggaaaccaggaaaaggatccgctactcaaaaaccaagaatttaaaggagtttcttaaatttcgaccttgtttctgaagctcacttttcagtgccattgatgtgagatgtgctggagtggctattaacctttttttcctaaagattattgttaaatagatattgtggtttggggaagttgaattttttataggttaaatgtcattttagagatggggagagggattatactgcaggcagcttcagccatgttgtgaaactgataaaagcaacttagcaaggcttcttttcattattttttatgtttcacttataaagtcttaggtaactagtaggatagaaacactgtgtcccgagagtaaggagagaagctactattgattagagcctaacccaggttaactgcaagaagaggcgggatactttcagctttccatgtaactgtatgcataaagccaatgtagtccagtttctaagatcatgttccaagctaactgaatcccacttcaatacacactcatgaactcctgatggaacaataacaggcccaagcctgtggtatgatgtgcacacttgctagactcagaaaaaatactactctcataaatgggtgggagtattttggtgacaacctactttgcttggctgagtgaaggaatgatattcatatattcatttattccatggacatttagttagtgctttttatataccaggcatgatgctgagtgacactcttgtgtatatttccaaatttttgtacagtcgctgcacatatttgaaatcatatattaagactttccaaagatgaggtccctggtttttcatggcaacttgatcagtaaggatttcacctctgtttgtaactaaaaccatctactatatgttagacatgacattctttttctctccttcctgaaaaataaagtgtgggaagagacaagaaaaaaaaaa

SEQ ID NO: 21 - DNA, 546 bp, Rattus norvegicus
  misc_feature (1)..(546): cDNA for alpha2u globulin
atgaagctgttgctgctgctgctgtgtctgggcctgaccctggtctgtggccatgcagaagaggctaattccgagagagggaacctcgatgtggacaagctcaatggggattggttttctattgtcgtggcctctaacaaacgagaaaagattgaagagaatggcagcatgagagtttttatgcagcacatcgatgtcttggagaattccttaggcttcaagttgtgtattaaggaaaatggagagtgcaggaaactatattcagttgcctacaaaacgccaaagattggcgaatattttcttgagtatgacggagggaatacatttactatacttaagacagactatgaaagatatgtcatgtttcatctcgttaatgtcaacaatggggaagccttccagctgatggagctctatggtagaacaaaggatctgagttcagacatcaaggaaaagtttgcaaaactatgtgaggcgcatggaatcactagggacaatatcattgacctaaccaagactgatcgctgtctccaggcccgaggatga

SEQ ID NO: 22 - DNA, 1296 bp, Rattus norvegicus
  misc_feature (1)..(1296): cDNA for actin, beta
ggggtcgagtccgcgtccacccgcgagtacaaccttcttgcagctcctccgtcgccggtccacacccgccaccagttcgccatggatgacgatatcgctgcgctcgtcgtcgacaacggctccggcatgtgcaaggccggcttcgcgggcgacgatgctccccgggccgtcttcccctccatcgtgggccgccctaggcaccagggtgtgatggtgggtatgggtcagaaggactcctacgtgggcgacgaggcccagagcaagagaggcatcctgaccctgaagtaccccattgaacacggcattgtcaccaactgggacgatatggagaagatttggcaccacactttctacaatgagctgcgtgtggcccctgaggagcaccctgtgctgctcaccgaggcccctctgaaccctaaggccaaccgtgaaaagatgacccagatcatgtttgagaccttcaacaccccagccatgtacgtagccatccaggctgtgttgtccctgtatgcctctggtcgtaccactggcattgtgatggactccggagacggggtcacccacactgtgcccatctatgagggttacgcgctccctcatgccatcctgcgtctggacctggctggccgggacctgacagactacctcatgaagatcctgaccgagcgtggctacagcttcaccaccacagctgagagggaaatcgtgcgtgacattaaagagaagctgtgctatgttgccctagacttcgagcaagagatggccactgccgcatcctcttcctccctggagaagagctatgagctgcctgacggtcaggtcatcactatcggcaatgagcggttccgatgccccgaggctctcttccagccttccttcctgggtatggaatcctgtggcatccatgaaactacattcaattccatcatgaagtgtgacgttgacatccgtaaagacctctatgccaacacagtgctgtctggtggcaccaccatgtacccaggcattgctgacaggatgcagaaggagattactgccctggctcctagcaccatgaagatcaagatcattgctcctcctgagcgcaagtactctgtgtggattggtggctctatcctggcctcactgtccaccttccagcagatgtggatcagcaagcaggagtacgatgagtccggcccctccatcgtgcaccgcaaatgcttctaggcggactgttactgagctgcgttttacaccctttctttgacaaaacctaacttgcgcaaaaaaaaaaaaaaaaaaaaaaaaaaaaaa

SEQ ID NO: 23 - DNA, 1477 bp, Rattus norvegicus
  misc_feature (1)..(1477): cDNA for tubulin, alpha 4A
cgactctacgtgagacgtacatcccaaactcatcatgcgtgagtgcatttcagtccatgtgggccaggcaggtgtccagatgggcaatgcctgctgggagctctactgcctggaacatgggattcagcctgatgggcagatgcctagcgataagaccattggtggaggggatgactccttcaccaccttcttctgtgaaactggagctggaaaacatgtgcctcgggcagtctttgtggatctggagcctactgtaattgatgagatccgaaatggcccgtaccgccagctcttccacccagaacagcttatcactgggaaggaagatgcagccaacaactatgcccgtggtcactacaccattggcaaggagatcatcgacccagtgctggaccggatccgcaagctgtctgatcagtgtacaggactccagggctttctggtgttccacagctttggaggaggcactggctctggcttcacctctctgctgatggaacggctttctgttgactatggcaagaagtccaagctggagttctccatctacccagccccacaagtgtccacggctgtagtggagccctataactccatcctgaccacccataccaccctggaacactcggactgtgccttcatggtggacaacgaggccatttatgacatctgtcgccgtaacctggatattgagcgcccaacctataccaacctcaaccgcctcatcagccaaattgtctcctccatcacagcttccttgcgctttgatggggccctcaatgtggacctgacagagttccagaccaacctggtaccctaccctcgcatccacttccccttggctacctacgcaccagtcatttctgcagagaaggcctaccatgagcagctgtcagtggcagagatcaccaatgcctgttttgagcctgccaaccagatggtgaaatgtgaccctcggcatggcaagtacatggcctgctgcctgctgtaccggggagatgtggtgcccaaggatgtgaacgctgccatcgctgccatcaagacgaagcgcagcatccagtttgtggactggtgccccacaggcttcaaggtcggtatcaactaccagcctcccaccgtggtgcctgggggcgacctggccaaggtgcagcgtgcagtgtgcatgctgagcaacacaaccgccattgctgaagcctgggcccgcctggaccacaagtttgacttgatgtatgccaagagggcttttgtacactggtatgtgggtgagggaatggaggagggggagttctccgaggctcgagaggatatggctgccctggagaaagattatgaggaagtaggcatcgactcctatgaggacgaggatgagggagaagaatagacagctacttggagccctgttcactgtgtttattgcaaaatcctttcgaaataaacagtctccttgcaaaaaaaaaaaaaaaaaaaaaaaaaaaaa

SEQ ID NO: 24 - DNA, 1514 bp, Homo sapiens
  misc_feature (1)..(1514): cDNA for tubulin alpha 4a
agttctcactgagacctgtcaccccgactcaacgtgagacgcaccgcccggactcaccatgcgtgaatgcatctcagtccacgtggggcaggcaggtgtccagatgggcaatgcctgctgggagctctattgcttggaacatgggattcagcctgatgggcagatgcccagtgacaagaccattggtggaggggacgactccttcaccaccttcttctgtgaaactggtgctggaaaacacgtaccccgggcagtttttgtggatctggagcctacggtcattgatgagatccgaaatggcccataccgacagctcttccacccagagcagctcatcactgggaaagaggatgctgccaacaactatgcccgtggtcactataccattggcaaggagatcattgacccagtgctggatcggatccgcaagctgtctgaccagtgcacaggacttcagggcttcctggtgttccacagctttggtgggggcactggctctggcttcacctcactcctgatggagcggctctctgttgactatggcaagaaatccaagctggaattctccatctacccagccccccaggtgtctacagccgtggtcgagccctacaactctatcctgaccacccacaccaccctggagcactcagactgtgccttcatggtggacaacgaagcaatctatgacatctgccgccgcaacctagacatcgagcgcccaacctacaccaacctcaatcgcctcattagccaaattgtctcctccatcacagcttctctgcgctttgacggggccctcaatgtggacctgacagagttccagaccaacctggtgccctaccctcgcatccacttccccctggccacctatgcaccagtcatctctgcagaaaaggcataccacgagcagctgtcggtggcagagatcaccaatgcctgctttgagcctgccaaccagatggtaaagtgtgatccccggcacggcaagtacatggcctgctgcctgctgtaccgtggagatgtggtgcccaaggatgtcaacgctgccattgccgccatcaagaccaagcgcagcattcagtttgtggactggtgccccacaggcttcaaggttggtatcaactaccagcctcccactgtggtgcctgggggtgacctggccaaggtgcagcgtgccgtgtgcatgctgagcaacacgaccgccatcgccgaggcctgggcccgcctggaccacaagttcgacctgatgtatgccaagagggcgtttgtgcactggtatgtgggtgagggcatggaggagggtgagttctccgaggcccgtgaggatatggctgccctggagaaggattatgaggaggtgggcatcgactcctatgaggacgaggatgagggagaagaataaagcagctgcctggagcctattcactatgtttattgcaaaatcctttcgaaataaacagtttccttgcacggttaaaaaaaaaaaaaaaaaaaaaaaaaaaaaaaaaaaa

SEQ ID NO: 25 - DNA, 1885 bp, Rattus norvegicus
  misc_feature (1)..(1885): cDNA for progesterone receptor membrane component 1
gcaaaattaatttattggccactgttcgcttagagggcggaggaagccgactgtttcggtctctgcataacaggcccaaacctttgctccagagatcatggctgccgaggatgtggtggcgactggcgccgaccccagcgagctggagggcggcgggctgcttcaagagattttcacgtcgcctctcaacctgctgctccttggcctctgcatcttcctgctctacaagatcgttcgcggggaccagcccggtgccagtggggacaacgacgacgacgagccgcccccgctgcctcgcctcaagccgcgtgacttcacccctgccgaactaaggcgatacgatggagtccaggacccgcgcattcttatggccatcaacggcaaggtgttcgacgtgaccaaaggccgcaagttctatgggccggagggaccatacggggtctttgctggaagagatgcatccaggggccttgccacattttgcctggacaaagaagcactgaaggatgagtatgatgacctttctgacctcactcctgcccagcaggagaccctgaatgactgggactctcagttcagttcaccttcaagtaccatcacgtggggaaaactgcttgaaggagcggaggagccgattgtgtactcggatgatgaagaacaaaagatgaggctgctcggaagagtgactgaagcagtcagtggagcatatctatttttgtattttgcaaaatcatttgtaacattccagtctgtctttacaacatggtgatttcaatatttagagaagttttgaacttgtaagttttttaagtaacatcactagtgacaccgataaaatcaactcgtcttagaatgcatgatgtgtttgtacgtcacaaatccagaaagtgaactttagttctctagatccaaatggtaaagctgtttctcttctatctgtagttaaaactgaattacaattttattttcctgagagggaaaacttgggttatttccacaaactggccttttttcaagtgcacacacacacagtggccccaaatggtcaatgtactaagaatgaagagagaaggtcttagccatgctatagtctgaaaacaagcccattttacccaacagacttaactgcatgatttttgttttatctacctctaaagcaaaactgcagtgttccaaagtctgtggtattgattcaaaacagaagtccagtaacaaaatgaaaactcaataatgggtttagttggggcaaacacattgcctgtgttcatggactgatttatcatccctgtccccacgtggacactcccacacacagtaactctcacacacctggtaattggcagttggaactacaacagaaatctgaaaattcaaggtagaactttgcaaaagaaaaatctgttgcttgtagcagggcaatggttatgtggttattggccaaatgtaaaatttgagagcaatatacaggacaggaacaggtgtgcgcttttcaaaagcttccactgacatctctgaagaatgagaaaacactacatggcactagctgggtgattttgaaaggaaaatacattcctaaggtaacagttaatcctctgttgttacaaaactcgtagtcgcttcaaaaatagtagtcaggtgtctaggtcttggataatactcttggttaatactaaacctagcttaagtagactctgcagttgtatacatttgttaaaagtattatctgaacaactagtgaggtttcagactcttgtaattgtagttaaatagtcattgtattttcttgtgagctgtgtttaatggttttacctcaaatcagaaaaaaatgaagtgcttgggtcagttaataaaacggttttgcccagtaaaaaaaaaaaaaaaaaaa

SEQ ID NO: 26 - DNA, 1928 bp, Homo sapiens
  misc_feature (1)..(1928): cDNA for progesterone receptor membrane component 1
ccggctagtctttggccgccgccgaaccccgcgcgccactcgctcgctcagagggaggagaaagtggcgagttccggatccctgcctagcgcggcccaacctttactccagagatcatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattaaagcattcagtggaagtatatctatttttgtattttgcaaaatcatttgtaacagtccactctgtctttaaaacatagtgattacaatatttagaaagttttgagcacttgctataagttttttaattaacatcactagtgacactaataaaattaacttcttagaatgcatgatgtgtttgtgtgtcacaaatccagaaagtgaactgcagtgctgtaatacacatgttaatactgtttttcttctatctgtagttagtacaggatgaatttaaatgtgtttttcctgagagacaaggaagacttgggtatttcccaaaacaggtaaaaatcttaaatgtgcaccaagagcaaaggatcaacttttagtcatgatgttctgtaaagacaacaaatccctttttttttctcaattgacttaactgcatgatttctgttttatctacctctaaagcaaatctgcagtgttccaaagactttggtatggattaagcgctgtccagtaacaaaatgaaatctcaaaacagagctcagctgcaaaaaagcatattttctgtgtttctggactgcactgttgtccttgccctcacatagacactcagacaccctcacaaacacagtagtctatagttaggattaaaataggatctgaacattcaaaagaaagctttggaaaaaaagagctggctggcctaaaaacctaaatatatgatgaagattgtaggactgtcttcccaagccccatgttcatggtggggcaatggttatttggttattttactcaattggttactctcatttgaaatgagggagggacatacagaataggaacaggtgtttgctctcctaagagccttcatgcacacccctgaaccacgaggaaacagtacagtcgctagtcaagtggtttttaaagtaaagtatattcataaggtaacagttattctgttgttataaaactatacccactgcaaaagtagtagtcaagtgtctaggtctttgatattgctcttttggttaacactaagcttaagtagactatacagttgtatgaatttgtaaaagtatatgaacacctagtgagatttcaaacttgtaattgtggttaaatagtcattgtattttcttgtgaactgtgttttatgattttacctcaaatcagaaaacaaaatgatgtgctttggtcagttaataaaaatggttttacccactaaaaaaaaaaaaa

SEQ ID NO: 27 - DNA, 837 bp, Rattus norvegicus
  misc_feature (1)..(837): cDNA for peroxiredoxin 5
cagaatcatctgcgggtgggctgcggcggcggggcgggcatggtccagctgaggttttgcgtcctaggcagcatagccggatcggtgctccgtgcatcggctacttggacgtgcgtggcaggcagagcaggccggaaaggagcaggttgggagtgtggtggggcccgcagcttcagcagtgccgcggtgactatggccccgatcaaggtgggagacaccattccctcagtggaggtatttgaaggggaacctggaaagaaggtgaacttggcagagctgttcaaggacaagaaaggtgttttgtttggagtccctggggcatttacacctggctgttccaagacccatctgcctgggtttgtggagcaagccggagctctgaaggccaagggagcacaagtggtggcctgtctgagtgttaatgatgccttcgtgactgcagagtggggtcgagcccaccaggcagaaggcaaggttcagctcctggctgaccccactggagcttttggaaaggagacagatttactactagatgattctttggtgtctctctttgggaatcgtcggctaaaaaggttctccatggtgatagacaagggcgtagtaaaggcactgaacgtggagccggatggcacaggcctcacctgcagcctggcccccaacatcctctcacaactctgaggccctgaccagaatgtcctctgactctcccatctcctccacccagctctgggccaaaggcccagtacctccttacctgagggccactggaatggaaccttgacaatatttctgcaataaacagtttaatttgtgaaaaaaaaaaaaaaaaaaaaa

SEQ ID NO: 28 - DNA, 959 bp, Homo sapiens
  misc_feature (1)..(959): cDNA for peroxiredoxin 5, variant 1
gcagtggaggcggcccaggcccgccttccgcagggtgtcgccgctgtgccgctagcggtgccccgcctgctgcggtggcaccagccaggaggcggagtggaagtggccgtggggcgggtatgggactagctggcgtgtgcgccctgagacgctcagcgggctatatactcgtcggtggggccggcggtcagtctgcggcagcggcagcaagacggtgcagtgaaggagagtgggcgtctggcggggtccgcagtttcagcagagccgctgcagccatggccccaatcaaggtgggagatgccatcccagcagtggaggtgtttgaaggggagccagggaacaaggtgaacctggcagagctgttcaagggcaagaagggtgtgctgtttggagttcctggggccttcacccctggatgttccaagacacacctgccagggtttgtggagcaggctgaggctctgaaggccaagggagtccaggtggtggcctgtctgagtgttaatgatgcctttgtgactggcgagtggggccgagcccacaaggcggaaggcaaggttcggctcctggctgatcccactggggcctttgggaaggagacagacttattactagatgattcgctggtgtccatctttgggaatcgacgtctcaagaggttctccatggtggtacaggatggcatagtgaaggccctgaatgtggaaccagatggcacaggcctcacctgcagcctggcacccaatatcatctcacagctctgaggccctgggccagattacttcctccacccctccctatctcacctgcccagccctgtgctggggccctgcaattggaatgttggccagatttctgcaataaacacttgtggtttgcggccaaaaaaaaaaaaaaaaaaaaaaaaaaaaaaaaaaaaaaaaaaaaaaaaaaaaaaaaaaaaaaaaaaaaaaaaaaaa

SEQ ID NO: 29 - DNA, 5270 bp, Rattus norvegicus
  misc_feature (1)..(5270): cDNA for vinculin
ctccgagtcagtctctcgccgccggctcccggccctacggttcccggttttctgttgcctgcggttcgccgccccgccagccgccgcgatgccggtgtttcacacgcgcacgatcgagagcatcctggagccggtggcgcagcagatctctcacctggtgattatgcacgaggagggcgaggtggacggcaaagccattcctgacctcaccgcgcccgtggccgctgtgcaggcggccgtcagcaacctcgtccgggttgggaaagagaccgttcagaccactgaggatcagattttgaagagagatatgccgccagcatttattaaggtcgagaatgcttgcacgaagcttgttcaggcagctcagatgcttcagtcagacccatactctgttcctgcgcgggattacctcatcgacggctccaggggaatcctctctggaacgtctgacctcctgcttacctttgatgaggctgaggttcgaaaaattataagagtttgcaaagggattttggaatacctcacagtggcagaggtagtggagactatggaagacttggtcacttacacaaagaaccttgggcccggaatgacgaagatggccaaaatgatcgatgagagacagcaggagttgacgcaccaggaacaccgtgtgatgctggtgaactcaatgaacactgtcaaagagctgcttcctgttctcatttcagctatgaagatttttgttacaaccaaaaactcaaaaaaccaaggaatagaagaagctctgaaaaatcgaaattttactgtaggaaagatgagtgctgagattaacgagatcattcgtgtgttacaactcacctcctgggatgaggacgcctgggccagcaaggacactgaagccatgaagagagcgctggcctccatagactcgaaactgaaccaggccaaaggttggctccgtgaccccaacgcttccccaggggacgccggtgagcaggccatcaggcagatcttggacgaagctggaaaagttggtgaactttgtgcaggcaaggaacgcagggagatcctaggaacctgcaagatgctagggcagatgactgaccaagtggctgacctccgagccagaggacaaggagcctcgccagtggccatgcagaaggcccagcaagtgtctcaggggctcgacgtgcttactgcgaaagtggaaaatgcagctcgaaagctggaagccatgacgaactcaaagcagagtattgcgaagaagattgacgctgcgcagaattggctggcagatcccaatggtggacctgaaggagaagaacagatccgaggggctttggctgaagctcggaagattgctgagttatgtgatgatcctaaggagagggacgacatcctccgctctcttggagagatagcggctctgacctctaaattaggagacttgcgaagacaggggaaaggagactccccagaggctcgagccttggctaaacaagtggcaaccgcgctacagaacctgcagaccaaaaccaatagggctgtggccaacagcaggcctgccaaggcagccgtccacctggaaggcaagattgagcaggcacagcggtggattgataaccctacagtggatgaccgaggagtcggtcaggctgccatccgtggacttgtggctgaagggcatcggctggccaatgtcatgatgggaccttatcgccaagatcttctcgccaagtgtgaccgtgtagaccagctagcagctcagctggctgacctggctgcccgaggggagggggagagtcctcaggcgagagcgcttgcttctcagcttcaggactccttaaaggatcttaaaacccagatgcaggaagctatgactcaagaggtgtccgacgttttcagcgatactacaactcccatcaagctgttggcagtggcggccactgctcctcctgatgcgcccaatagggaagaggtatttgacgagagagcagccaactttgaaaaccattcaggaaggcttggggccacggcagagaaggcagctgctgttggtgctgccaataaatcgacagtggaaggcattcaggcatccgtgaagacagcccgagaactcactccccaggtgatctcagctgctcgaatcttactgaggaaccctggtaaccaagctgcgtatgaacattttgagaccatgaagaaccagtggattgataatgttgagaaaatgacagggctggtggacgaggccattgataccaaatctctgttggatgcttctgaagaagcaattaaaaaagacctggacaagtgtaaggtagccatggccaatattcagcctcagatgctggtcgctggagcaaccagtattgctcgtcgggccaaccggattctgctggttgctaagagggaggtagagaactctgaggatccgaagttccgagaggctgtgaaagctgcctctgatgaactgagcaaaacaatctcccccatggttatggatgccaaggctgtggctggaaacatctctgaccctggcctgcaaaagagcttcctggactcaggatatcggatcctgggagctgtggccaaagttagagaagccttccaacctcaggagcctgacttcccgcctcctccaccagaccttgaacagctacgtctaactgatgagctggctcctcccaaaccacctctgcctgagggtgaagtccctccacccaggccacccccaccagaagagaaggatgaagagttccctgagcagaaagccggggaggtgattaaccagccgatgatgatggccgccaggcagctccacgatgaagctcggaagtggtctagcaagggcaatgacatcatcgcagcagccaagcgcatggctctgctgatggcggagatgtctcggctggtaagagggggcagtggtaccaagcgggcacttattcagtgtgccaaggatatcgccaaggcctctgatgaggtgacgaggttggccaaggaggttgccaagcagtgcacagataagcggattagaaccaatctcttacaggtctgtgagcgaatcccaactataagcacccagctcaaaatcctgtccacagtgaaggccaccatgctgggccggaccaacatcagtgacgaggagtctgagcaggccacagagatgctggttcataatgcccagaacctcatgcagtctgtgaaagagactgtgcgagaggccgaagctgcttcaatcaagattcgaacagacgccggatttactctgcgctgggtcagaaagactccctggtaccagtaggcacctggtcagacctggctggtacacagacctctgctaatgaggaggtgaccatcttgagcttcagaagccattcagagttgccaggggtggaaaatcaatccctggtttcacacaccagaaaggaatggggcctcttcacatagaaaaaacatttatactcttgtcatggacactttgaagtgtctctctacaaaccctgtactttcagttactctgtgcactctcccaaggattttacataagctcagaataagcgtgaacactagcaggaagccctgctctgccctgggacatttctctctgtgtttatggttcactggctgcctttcccccctttaggctctcgagacctagaactgaggcgagtcagtaaataactgtgcttccaaagtgattttgggtttcccctttgctgctgaccctgcccatcttccctgggctgtgcatcactgctgcaggaaaaggtggtctctggggagttccagcttgcacagcctggatccatttcccactttctccccatgtattcctagttccgcacaatggaaaaccaactttcactgttaggaggaaatcactcttctgtctttaccatgcccctcactagtatgtgttatggaaacttcccttctcacatcctgccttgcttggttgccaaggtaaccatcatgtaaacacactggtgctctctgccactgagggggcagagtagctgggtgtgatcctgcccatcctcccagtggggccactcccaggttttctatgcttgtcactgccagtagaggtctgtgctgaggccttagcattcatccttagcttttactttcttttttaatttctgagctgaaatgctgcatttacttttaaccaaatcatcctaggcttgggcagtcttcctctatgtgcttcttaaacaagactttaaagatcataggtgtgtgtgtctgcaatctctccatcccccactttttttttttttttttttttaattttgagactgggtctctctatgttgtccaggctagtcttgaacttctggattcaagtcatctacttgtgtcagcctcttagctcctaccaccacacttgactttgcttgtaactttgaaaagtccattcaaaattaagctcttaagagactgaatggaaaggcaattttgtctgaaggatatttcctatgtaagggagaatagcatttgcagaatataattctggtgctgctagggaaaaatcagtaggaagttatagttcccagttgcttttaaccaactacaaccttctctcaatataaagtattcaagaataaagagtatggtatctacttatcagaaaggcatgtttcctattgggcaaagttagtgaaaaagtgactttactcatttttgcatttacctcggctgtataagcatttcctagcgcatggatgcttcttccagaaatcaagaaccaggtgaatacaggactaagaccttcctggatgttcttcccacatctagtatgttgaccccaacactgaacttgcaaatcttaagttgaccctgaatactcaggcttcccctatttcctttcagctgataacagattcttttgaaagctctcagcagatctgtatagttgcttacctgatataaatgcatatcaatgcctttaaagtatgaatctatgccaaagatcatcttttgttttactaaagattacttagaggaattaagaaaaatgtttgctctccaagttcttccagttctggagacatatgtccgccagatgtgcttttctcaagtgtcagtgctcacagtacaaggaggcaaaaggaaacagtccctgaacgacgtgttagagatgccaccactaaaaactacatttataagctaggacttacatgcaaatgttttctgcctcttctttcgttctgtttaaaacaataaagtacatttatatgaataatgctctaaatgaggttattttactaacgacttta

SEQ ID NO: 30 - DNA, 5647 bp, Homo sapiens
  misc_feature (1)..(5647): cDNA for vinculin
gtagtcgctgcacagtctgtctcttcgccggttcccggccccgtggatcctacttctctgtcgcccgcggttcgccgccccgctcgccgccgcgatgccagtgtttcatacgcgcacgatcgagagcatcctggagccggtggcacagcagatctcccacctggtgataatgcacgaggagggcgaggtggacggcaaagccattcctgacctcaccgcgcccgtggccgccgtgcaggcggccgtcagcaacctcgtccgggttggaaaagagactgttcaaaccactgaggatcagattttgaagagagatatgccaccagcatttattaaggttgagaatgcttgcaccaagcttgtccaggcagctcagatgcttcagtcagacccttactcagtgcctgctcgagattatctaattgatgggtcaaggggcatcctctctggaacatcagacctgctccttaccttcgatgaggctgaggtccgtaaaattattagagtttgcaaaggaattttggaatatcttacagtggcagaggtggtggagactatggaagatttggtcacttacacaaagaatcttgggccaggaatgactaagatggccaagatgattgacgagagacagcaggagctcactcaccaggagcaccgagtgatgttggtgaactcgatgaacaccgtgaaagagttgctgccagttctcatttcagctatgaagatttttgtaacaactaaaaactcaaaaaaccaaggcatagaggaagctttaaaaaatcgcaattttactgtagaaaaaatgagtgctgaaattaatgagataattcgtgtgttacaactcacctcttgggatgaagatgcctgggccagcaaggacactgaagccatgaagagagcattggcctccatagactccaaactgaaccaggccaaaggttggctccgtgaccctagtgcctccccaggggatgctggtgagcaggccatcagacagatcttagatgaagctggaaaagttggtgaactctgtgcaggcaaagaacgcagggagattctgggaacttgcaaaatgctagggcagatgactgatcaagtggctgacctccgtgccagaggacaaggatcctcaccggtggccatgcagaaagctcagcaggtatctcagggtctggatgtgctcacagcaaaagtggaaaatgcagctcgcaagctggaagccatgaccaactcaaagcagagcattgcaaagaagatcgatgctgctcagaactggcttgcagatccaaatggtggaccggaaggagaagagcagattcgaggtgctttggctgaagctcggaaaatagcagaattatgtgatgatcctaaagaaagagatgacattctacgttcccttggggaaatatctgctctgacttctaaattagcagatctacgaagacaggggaaaggagattctccagaggctcgagccttggccaaacaggtggccacggccctgcagaacctgcagaccaaaaccaaccgggctgtggccaacagcagaccggccaaagcagctgtacaccttgagggcaagattgagcaagcacagcggtggattgataatcccacagtggatgaccgtggagtcggtcaggctgccatccgggggcttgtggccgaagggcatcgtctggctaatgttatgatggggccttatcggcaagatcttctcgccaagtgtgaccgagtggaccagctgacagcccagctggctgacctggctgccagaggggaaggggagagtcctcaggcacgagcacttgcatctcagctccaagactccttaaaggatctaaaagctcggatgcaggaggccatgactcaggaagtgtcagatgttttcagcgataccacaactcccatcaagctgttggcagtggcagccacggcgcctcctgatgcgcctaacagggaagaggtatttgatgagagggcagctaactttgaaaaccattcaggaaagcttggtgctacggccgagaaggcggctgcggttggtactgctaataaatcaacagtggaaggcattcaggcctcagtgaagacggcccgagaactcacaccccaggtggtctcggctgctcgtatcttacttaggaaccctggaaatcaagctgcttatgaacattttgagaccatgaagaaccagtggatcgataatgttgaaaaaatgacagggctggtggacgaagccattgataccaaatctctgttggatgcttcagaagaagcaattaaaaaagacctggacaagtgcaaggtagctatggccaacattcagcctcagatgctggttgctggggcaaccagtattgctcgtcgggccaaccggatcctgctggtggctaagagggaggtggagaattccgaggatcccaagttccgtgaggctgtgaaagctgcctctgatgaattgagcaaaaccatctccccgatggtgatggatgcaaaagctgtggctggaaacatttccgaccctggactgcaaaagagcttcctggactcaggatatcggatcctgggagctgtggccaaggtcagagaagccttccaacctcaggagcctgacttcccgccgcctccaccagaccttgaacaactccgactaacagatgagcttgctcctcccaaaccacctctgcctgaaggtgaggtccctccacctaggcctccaccaccagaggaaaaggatgaagagttccctgagcagaaggccggggaggtgattaaccagccaatgatgatggctgccagacagctccatgatgaagctcgcaaatggtccagcaagccgggcatcccagccgctgaggtgggtataggtgttgtagctgaggcagatgcggccgatgctgctggcttccctgtcccccctgacatggaagacgattacgaacctgagctgctgttaatgccatccaatcagccggtcaaccagcccattctggccgcggctcagtccttgcatcgggaagctaccaagtggtctagtaagggcaatgacatcattgcagcagccaagcgcatggctctgctgatggctgagatgtctcggctggtaagagggggcagtggtaccaagcgggcactcattcagtgtgccaaggacatcgccaaggcctcagatgaggtgactcggttggccaaggaggttgccaagcagtgcacagataaacggattagaaccaacctcttacaggtatgtgagcgaatcccaaccataagcacccagctcaaaatcctgtccacagtgaaggccaccatgctgggccggaccaacatcagtgatgaggagtctgagcaggccacagagatgctggttcacaatgcccagaacctcatgcagtctgtgaaggagactgtgcgggaagctgaagctgcttcaatcaaaattcgaacagatgctggatttacactgcgctgggttagaaagactccctggtaccagtaggcacctggctgagcctggctggcacagaaacctctactaaaaagaaggaaaatgatctgagtcccaggagctgcccagagttgctgggagctgaaaaatcacatcctggcctggcacatcagaaaggaatgggggcctcttcaaattagaagacatttatactcttttttcatggacactttgaaatgtgtttctgtataaagcctgtattctcaaacacagttacacttgtgcaccctctatcccaataggcagactgggtttctagcccatggacttcacataagctcagaatccaagtgaacactagccagacactctgctctgcccttgttccctaggggacacttccctctgtttctctttccttggctcccattcactcttccagaatcccaagacccagggcccaggcaaatcagttactaagaagaaaattgctgtgcctcccaaaattgttttgagctttccatgttgctgccaaccataccttccttccctgggctgtgctacctgggtccttttcagaagtgagctttgctgctacaggggaaggtggcctctgtggagccccagcatatgggggcctggattcatttcctgcccttcctcagtttaatccttctagtttcccacaatataaaactgtacttcactgtcaggaagaaatcacagaatcatatgattctgcttttaccatgcccctgagcaatgtctgtgctagggaaacttcccgtcccatatcctgcctcagcccgccaaggtagccatcccatgaacacactgtgtcctggtgctctctgccactggaagggcagagtagccagggtgtggccctgccatcttcccagcagggccactcccggcactccatgcttagtcactgcctgcagaggtctgtgctgaggccttatcattcattcttagctcttaattgttcattttgagctgaaatgctgcattttaattttaaccaaaacatgtctcctatcctggtttttgtagccttcctccacatcctttctaaacaagattttaaagacatgtaggtgtttgttcatctgtaactctaaaagatcctttttaaattcagtcctaagaaagaggagtgcttgtcccctaagagtgtttaatggcaaggcagccctgtctgaaggacacttcctgcctaagggagagtggtatttgcagactagaattctagtgctgctgaagatgaatcaatgggaaatactactcctgtaattcctacctccctgcaaccaactacaaccaagctctctgcatctactcccaagtatggggttcaagagagtaatgggtttcatatttcttatcaccacagtaagttcctactaggcaaaatgagagggcagtgtttcctttttggtacttattactgctaagtatttcccagcacatgaaaccttattttttcccaaagccagaaccagatgagtaaaggagtaagaaccttgcctgaacatccttccttcccacccatcgctgtgtgttagttcccaacatcgaatgtgtacaacttaagttggtcctttacactcaggctttcactatttcctttataatgaggatgattattttcaaggccctcagcatatttgtatagttgcttgcctgatataaatgcaatattaatgcctttaaagtatgaatctatgccaaagatcacttgttgttttactaaagaaagattacttagaggaaataagaaaaatcatgtttgctctcccggttcttccagtggtttgagacactggtttacactttatgccggatgtgcttttctccaatatcagtgctcgagacacagtgaagcaaattaaaaaaaaaaaaaaaaaaaatccctgaatgatgattagagacatcaccgctaaaaaactacatttataagctaggatttgttatatgcaaatattttctgcctcttcttttgttctgtttaaaacaataaaatgcatttgtataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 1101 bp, Rattus norvegicus
  misc_feature (1)..(1101): cDNA for chemokine (C-X-C motif) ligand 2
actgcacctctgggcctccagcaagctccctcctgtgctcaagactccaaccactctttggtccagagccatggcccctcccactcgccagctcctcaatgctgtactggtcctgctcctcctgctggccaccaaccatcagggtacaggggttgttgtggccagtgagctgcgctgtcaatgcctgacgaccctaccaagggttgacttcaagaacatccagagcttgacggtgacccctccaggaccccactgcgcccagacagaagtcatagccactcttaaggatggtcatgaagtttgtctcaaccctgaagcccccttggttcagaggatcgtccaaaagatactgaacaaaggcaaggctaactgacctggaaaggaagaacatgggctcctgtacctcaacgggcagaatcaaagagaaaagaaacaaactgcacccaggaagcctggatcgtacctgatgtgcctcgctgtctgagtttatctatttatttatatatgtatttatttatttattttcagtgcctagatgttgttacatttactatgatatttaaagatatgcattggccagctcactgtagtatcttaagaggtcattttaatatgttgaagtttattgtaataatgttcaatgtgttcagtcagcattattttacttatgtagttggaaggtgatgcatttttaaatctatatttattactttctggggggggagggggagttgggtactgactacaccacctccacactgtgatagagattggggatgaggggggtgggggggcaaacagacgcagtcagagggctttcaaggcaggactgtgcctgtccacgtcattttctgtaagccccgagaagggcgggacgactgttatttctgtctccgtgtttctacactatgtgtacaacatttctgatgctgaatgttcaacaatcgtaatgtgaatatcccctggacattctatgtcttctctgtaaggcacagtgcctcgtttagcaattgttttgtcatgctttctcatgtcttgaagtggggacatttatttattcatgtacttttacaaataacaaaaaaaataaaaatttttact

SEQ ID NO: 32 - DNA, 1234 bp, Homo sapiens
  misc_feature (1)..(1234): cDNA for chemokine (C-X-C motif) ligand 2
gagctccgggaatttccctggcccgggactccgggctttccagccccaaccatgcataaaaggggttcgccgttctcggagagccacagagcccgggccacaggcagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgcccttacaggaacagaagaggaaagagagacacagctgcagaggccacctggattgcgcctaatgtgtttgagcatcacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtgagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatataaaaaaaaaaaaaaaaaaaaaaaaaaaaaaaaa

SEQ ID NO: 33 - DNA, 7500 bp, Rattus norvegicus
  misc_feature (1)..(7500): cDNA for protein tyrosine phosphatase, receptor type J
cggccgctccgcgttaggagcgctaggctccgccgtgtggccgccgccgccgccgctgccatgtccccggggaagcccggggcgggcggagcggggactaggcggaccggctggcggagaaggaggcggaggcgtcggctggaggcggagaccagggcgcccggcttcgggcacacggcggggcgcgtcccgggcacgttccagggcgcgcagggcatgaagcccgcggcgcgggagacgcggacacccccgcgctcgcccgggctccgctgggcgctgctgccgctgctgctgttgctacgccagggccaggttgtgtgcgcaggtgcggcgcccagtcctgtatttgacgttgaagctgtcaccagccctaccagtgtggtattaacgtggaaacacaatgattcagccacttcagaatataagataaatgagggaaacacgctgcggtatactgttaaaaaccagacatcatttaacatcacaggcttaagcccagctacttcctacaaattctccatcaccttgggaacagtcaatgagacctcaggaaagcccacatacaaaaatatcacaacagagccctggccagtgtcggatctccaagtcgcctacatcggtgtgacacaggctcttctcgcctggagcaatgcaaacggcaccgcttcttaccggatgcagattgtagagctgaccacaaattcatcaggtggtatttcagatctgaagccggggacccataagagcttggctgtccaaggatcgaatgagacccagcatgacttatgggtcacagaaggcgtctcggatgccaatggcaccgagggaagcccagtggccaacatatcccaactacataagaattctcttgcctctgcagacccaccctctgcccgggatccctccctcacagaggtcttgctcactgagctaaagcctgatactcagtacaaagtcaccatttattctcaagcagcagatggcacagaaggacagcctgggaacaaagtgtttaaaacaaatcccatccaagtttctgacatccgagctgtgaacatcagtgactcgaacatgaccctgacctggaaaagcaacaataatgagtcccatgcttcatttacctacaaaatatacgtggctgggggttccgactccatcaacgaaactgtcaatgagactcaggccgtcatccgtggactcagctccagtaccttgtacaacatcacagtgcttcctttcctgggtcagaccgcgggtataccaggcttcctccaagtgtacacttccccccgtcctgtttctgacttccgagtgacaaatgtcagcctgagggaaatcggtttggcttggagaagcaatgactcggagtcattcgagattttcatcacgcaggagggaagtgagaaacgttggaatgcttcgacgggagacctgagctatatcgttgaaaatttaaagcctgggaccagttaccaatttgaaatattcccacgaggaccaaatgggactgaagggccatcccagacagttgttggtagaactgactgcagtgctgtgactgacatccgcgtggtcagcgttagcaccactgaaatacagctggagtggcagaatacagacagtgcttctggatacacttaccatttagttctagagtctgagaatggctccatcaagaccaacagttctcagaaatggatcacagtggggggccttaccccaggcaccttatacaatgttacaatctttccagaagtggaccagatggagggcaactccagctctattacccagtacacaaggcccagcaatgtgtcctacattgaagtaaacaccaacaccaccgtggcagccatccagtggaagaacctggacgcagcctctgcttcctactcctactccgttcttatcttgaaggctggagatggcagcaatgtaaccagcagggtcagagacatcccttctgtcaccatccctgggctaatccccggcgtctcttacgaagtgaagatctttacaaagattaggaatacagaggttgggaatgaggtacctggtcagaagctgttctgtatggaacctgcacaagtggattccttacactgtgaagtggtcccgaaggagccagcgctggttctgaagtgggcctgcccccctggcatgaactcaggctttgaactgggggtcaggagtgatgcttgggacaatatgacacacttggagaactgcactttggacaatgacacagagtgcaggacagaagtcacctatttgaatttttctacctcgtacaacatcagcattgccaccttgtcatgtggaaagatggcattgcccacccagagcacctgcaccactggcatcacagacccacctcctccagatggatcccctaatattacatcggtcagtcacaattcagtaaaggtgaagttcagtggttttgaagccagccatggacctatcaaagcctatgctgtccttctcaccacaggggaagctggccaaccctccacagatgttttgaagtacacatatgaagatttcaaaaagggagcctccgatacttatgtgacatacctcataaggatagaagagaagggacagtctcagggcttgtctgaagcattgaactatgagattgatgtggggaaccaatccactaccctcggctactacaacgggaggctagagcctctgggctcctaccgggcttgcgttgctggctttaccaatattacctacaaccttcagaacgatggccttattaatggggatgagagctatgtatctttcagtccgtattcagaggccgtgtccttgcctcaagatccaggtgtcatctgcggagcggtgttcggatgtatctttggtgccctggccattgtggctgtgggaggcttcatcttctggagaaagaaaaggaaagatgccaagaataatgaagtgtccttttctcaaattaaatccaagttaatccgagtggagaattttgaggcctactttaagaaacagcaagccgactccaactgtgggtttgcagaggaatatgaggatctgaagctgattggaataagtttacctaaatatgcagcggaaatagctgaaaacagggggaagaaccgctacaacaacgtcctgccctatgatatttctcgagtcaaactttcagtccagacccattcgacagacgactacatcaatgccaactatatgcctggctaccattccaagaaagatttcattgccacacaaggacctttacccaacactttgaaagatttttggcgtatggtttgggagaaaaacgtatatgccattgttatgctgaccaaatgtgtggagcagggaaggaccaaatgtgaagagtactggccttccaagcaggctcaggactacggggacataactgtggcaatgacatcagaagttgttcttccggaatggaccatcagagattttgtggtgaaaaatatgcagagtagtgagagtcatcctctgcggcagttccatttcacctcctggcctgaccatggtgttcctgacaccaccgacctgctcatcaactttcggtacctggtccgggattacatgaagcagatcccccctgagtcaccaatcctggtgcattgcagtgctggggttggaaggacgggcactttcattgctattgatcgcctgatctatcagatagagaacgagaacaccgtggatgtgtatggcattgtctacgatcttcggatgcacaggcctctgatggtacagacagaggaccagtatgttttcctcaatcagtgtgtgttggatattatcagagcccagaaagactcaaaagttgacctcatctatcagaacacaaccgcaatgacaatctatgaaaacctcgagccagtgagcatgtttggaaagactaatggttacatcgcctagttccaaagcaaacccttctgaagctaaccagaccgtcacacccacagtgaaggagaacgccctgacggggacatgttttatgtgtctcatttctcaattctttgttctgttctgttagaactacttggagggcatggggctgtgcatgtaaaacatggcagataggcagttggggctctcaggggctgtgggcaggtggtgtgtaaatcagctgcattcctgattaccaatgggatgggttcgctttctgtttttctttccttgagaattgtggaaaaccaggaaaagtgagctatgtttttgttttccaagcaggtttgttctcctctctttctattcctcccccttctctctctccctcccctgcctctctctgtttttttttaagacttttatatgattcacatgctaaagccaggattatgttgggttggatatattttaagtatcagaggtctatttttacctactgtatctcggaatctagccgatggaaaagcatggctgtgggtgtttctcatgcagggaggggcacccggtgcctcttctgcgtgggtttctatttgaacatgtgccttttctgaattatgcttccacgggcaaaactcagtagatatctctatttttatattgaatctcagaattggaatatacagaatatttaaacagtagcttagtatcagaggttcgcctcctcagtaacattcctgtcccatcccattagaggaggctttctctgcggcccgcccttgacacacatttgtgctccatgttccttttccttcctttctttttttttttctcccctcccagttttcgccaaagaacttctgttggtgagattttcatcccattggttgggtgagaatagaagtttaaaatatcaccatgagactctctgtgggtgggagtggggaggaacagaggaatcaacataccatggtgtgtgcaccccttttcatcccatctcctatgttctctcatttctgtctttggtaagaaggatttttttaaaggtgcaatatgtgacgtagtggttcacgatgctctgggttcttagtaatgttttactcaggttgtcatttatgtgtgtatgtgtgtgtgtgtgtgtgtgtgtgtgtatgtatgtatgtatgtgtgttttatagcgtggtgatctgtgaacatttcagtgtgaactcagattaacccctttccccccaaagtccactggctttagtctctctgcagtgacacacatcagtatctactgtatatataaatactaaactcttacacaccagtcattcctatgaattgaggtgtacaaagtttgatttgtatcaaagatgtaattattatttttaaaacctctttcactacactgctgctgtaattactttgaatttttaaagaaaaatgtagattttttttgttttgtttttggcttcaggtgtgtattcaccaagagtttcagaatttatgtggttttattttattggtacataatctgcaaacttctcaaggcattaacatgacaagatttgtatctcttatttttatcttgtggctcaggttatatttgaagaagtttcaagtttattttctagtaggcagtgaagtatacataaggtcaatgaaggaatcaccaggtccctggagtcctgtggctgttttccacagttacacatcctgctgtatgtttgggaccatgggtattgtgttgaagatgagggcaggggtcttctccgaggacctggccactgtgagccctggggtaacctgtcctctgatgttctgcactctgttcctcgagtcaccggcactttgatggctgactggacctgatcatgagtgtttgtagggttagagaggctgaaccatgatgtactgaaagccctcgctgtggttcagcactgtagtgtgcgattcttgctgtgacccagcgctgctgtagcactgttgctgccgtggctcggcgctggggcacaacccctttccctttgcggtactgaaaggattgaaagaagaggtcatggcgctgtcatgctctgggggagaattttacttctcacatggaaactgcactttttgctaaatcctttgcattttgatgttcacttagtgtccagccctttaggatgggctggccagggtaggttggccccctggactcaacttcactggggaacttgacgctgcagttgtgaatcagggaagccatgctgtctccaggtatggggtgaagctgaacatgggctcattatttccaaatgccctgtctggtctgctcactaggatgtgctgactttgggcactatacagaggggcctgttctggcagtagttctgtgcaccgcctatacacctcagtctgtcaggaacggttgtagtgggtggtaaggggtccaatcccacgcagcgcctatgccctgcctctgctgccatggaaagtgcgaggaaaggacccgcccctaaaacagccctgtagaagattagagtccctcaaaaaaaaaaaagcaacagaatcatgaacacatcatatacaattgcaaaacacagaaaccttctccactcctacctgtatgggtctagaaagtcattaggcatcagccccagcaagccattcccagtcccaagaggaaggctagtgacaggtctggagttccaaatgggcaggtcacaagaagctcaccatgccattctacatccggaggaggactgcgtggtggagtttgcatagcaaagccattggaaagggaaaattgacaacagagggtaaggttgggcagagagagggggaaggtccttctcactgtttgccccaagccctcagtggaactgtgaatgaaagttggaggagtctggtgaaatcggaatgatggaactccctgctcagatccgatggcagcaccgagaaaagcagccagtgtggggtgtggtcagtgaaagggctacagactggtggccaactgctttgaaggacttaggccattgactgatgacttctaaggagaaagatttttccaccagagctgaatctagcaacaattggcaccactgggtgtctgttctctgaggccattttgccatctccagccagactttggcacaatggacctgctggctttgtgctttgtgaagttggaggctctggggggtgggagtggggaggagtgggggagggtcaactatgcagttggttcattcatacttgttttctgggatggagaagcctgggttttttgtttgtttgtttgtttttttgcaatgcttgattgcggcttgttgagctaaacaagcctggagactgaagctgattcacctcaaaacaaagttggaattctgcattttccagattgctgtgtagcaactgtgtattcaacacctgtagctttttctccctggggcacagaatagaagttgatgtggactctaaactgtacttttcttgtaactattttggaatgatgcacatttctaatgtctgttgtacttgtacagagtattgatgtaggttgctttttttttttaaaggaaaaatggcctgaaaaactttttttaaagacttacaaataccaaatataaaata

SEQ ID NO: 34 - DNA, 3193 bp, Homo sapiens
  misc_feature (1)..(3193): cDNA for protein tyrosine phosphatase, receptor type J
cagccccagccgcatgacgcgcggaggaggcagcgggagcagccgcgggagccgggaccgggtagccgcgcgctgggggtgggcgccgctcgctccgccccgcgaagcccctgcgcgctcagggacgcggcccccccgcggcagccgcgctaggctccggcgtgtggccgcggccgccgccgccgctgccatgtctccggggaagcccggggcgggcggagcggggacgaggcggaccggctggcggaggaggaggcgaaggagacggcaggaggcggcgacgacggtgcccgggctcgggcgcacggcggggcccgattcgcgcgtccggggcacgttccagggcgcgcggggcatgaagccggcggcgcgggaggcgcggctgcctccgcgctcgcccgggctgcgctgggcgctgccgctgctgctgctgctgctgcgcctgggccagatcctgtgcgcaggtggcacccctagtccaattcctgacccttcagtagcaactgttgccacaggggaaaatggcataacgcagatcagcagtacagcagaatcctttcataaacagaatggaactggaacacctcaggtggaaacaaacaccagtgaggatggtgaaagctctggagccaacgatagtttaagaacacctgaacaaggatctaatgggactgatggggcatctcaaaaaactcccagtagcactgggcccagtcctgtgtttgacattaaagctgtttccatcagtccaaccaatgtgatcttaacttggaaaagtaatgacacagctgcttctgagtacaagtatgtagtaaagcataagatggaaaatgagaagacaattactgttgtgcatcaaccatggtgtaacatcacaggcttacgtccagcgacttcatatgtattctccatcactccaggaataggcaatgagacttggggagatcccagagtcataaaagtcatcacagagccgatcccagtttctgatctccgtgttgccctcacgggtgtgaggaaggctgctctctcctggagcaatggcaatggcactgcctcctgccgggttcttcttgaaagcattggaagccatgaggagttgactcaagactcaagacttcaggtcaatatctcgggcctgaagccaggggttcaatacaacatcaacccgtatcttctacaatcaaataagacaaagggagaccccttgggcacagaaggtggcttggatgccagcaatacagagagaagccgggcagggagccccaccgcccctgtgcatgatgagtccctcgtgggacctgtggacccatcctccggccagcagtcccgagacacggaagtcctgcttgtcgggttagagcctggcacccgatacaatgccaccgtttattcccaagcagcgaatggcacagaaggacagccccaggccatagagttcaggacaaatgctattcaggtttttgacgtcaccgctgtgaacatcagtgccacaagcctgaccctgatctggaaagtcagcgataacgagtcgtcatctaactatacctacaagatacatgtggcgggggagacagattcttccaatctcaacgtcagtgagcctcgcgctgtcatccccggactccgctccagcaccttctacaacatcacagtgtgtcctgtcctaggtgacatcgagggcacgccgggcttcctccaagtgcacaccccccctgttccagtttctgacttccgagtgacagtggtcagcacgacggagatcggcttagcatggagcagccatgatgcagaatcatttcagatgcatatcacacaggagggagctggcaattctcgggtagaaataaccaccaaccaaagtattatcattggtggcttgttccctggaaccaagtattgctttgaaatagttccaaaaggaccaaatgggactgaaggggcatctcggacagtttgcaatagaactggatgatttgaacacctgcctggaattccatcatctgaaacagagttggcagataagaatggcccctatgccaaatttggctcattgtctgtttttgtaaataaagttttattggatcacagccatgttcatttgcttatgtactgtctatggctactttggtgctagaattgagtagtggcagcagagaacgtgtggcttgtgaagcctgagatatttattttctggccattacagagaatgtttgctgacctctgggctagatcagtggttttcaaattgtggtccagggctctctagggatccctaggttctttcaaggggtctccaaggtagtattatcataacaatactaaaatgttatttggctttttcattctcattctcttttggagttttccaactttgatccgatatgggatgacattgtctctcaggtctaatggaatatatgctgtggattcttacattttaaaaatttcttcattttaaatccctgatatagtaaatattaataaatataacctatgtaaatacaaattctctgagagccttaataacttttaagatggtaaagaagtcctgagagcaaaaagtttaagaacctctggtcaagatgtgttagaaattaactactgtgctgtgttgggcaggtggactgttcacagtctttcctaaacaacactgtgatgagatactttgttagttctagaaggctaattgaaattgataaaatcattttatagtcagctacggtggcctgcacccatcgtcccagctacttgagaggctgcggcaggaggattgcttggcccaacagttgcagactagcaccagcctgggagaccctgtctcaaaaaaaaatttttttttatagaaacagtttaaattattaaattatttctaggcctacaggagaaacttctgggaaatcctagagagtaatgttcattattgttgttttaaaattttgaaataattgttttttggaaaggttgaaaaacatacagtcttccttgatctaagattcaagatagttgaaatcttttatacagagggtagaataaagatatacctaatgtaaatgatgagttaatgggtgcagcacaccaacatggcacatgtatacatatgtaacaaacctgcacgttgtgcacatgtaccctagaacttaaagtataataaaaacatatatatatatataaaaaaaaaaaaaaaaaaaaa

SEQ ID NO: 35 - DNA, 4045 bp, Rattus norvegicus
  misc_feature (1)..(4045): cDNA for heat shock 70kD protein 1A
ccaggcatccttctctgagtttctatctttctctcgattgtaactcctctcttcgaccatcaataccatattcaaaaaggtcttgtctgcctccgatttcagctcagactaatttcagccactaatgtccctcaagagcccaaccccattttttcttggttgccaacacccaaatccagaattaaaactggggttcctgtgtggagagccaagaggagacctaaggatggtggcttccactagaccacgtctgtaaatcaatcaaacctaagaaaattcttggcccattaaataagaaccaactggaattccaggctcacctggaatctctacgccttcgatccagtttggaaaatttgaagtcgctgagcccctacgagtggggagctccaggaacatccaaactgagcaactggggttccccccaccccccaccccgcccctcccggcaactttgagcctcttctgggacagatcctctgattcctaaataagtccatgaggtcagagttagcactgccattgtaacgcggctggaggggggtcacgtcaccggacacgcccccaggcacctcccttgggtctcccaaggctaggaaggggaagttataacccttaactcgagccccataatcagaactgtgcgagtctgcgaacccccacaaatcacaaccaactgtccacaacacggagctagcagtgacctttcctgtccattccactcaggcctcagtaatgcgtcgccatagcaacagtgtcaacagcagcaccagcaggtcccgccaccctcccccacaggaatccgtcctttccagctaaccccagatctgtctggagagttctgaacaggggcggaaccctcaactcctattactcaaaggaggcggggaagctccacccgacgcgaaactgctggaagattcctggccccaaggcctcctcccgctcgctgattggcccatgggagggtgggcggggccggaggaggctccttaaaggcgcagggcggcgcgcaggacaccagattcctcctcttaatctgacagaatcagtttctggttccactcgcagagaagcagagaagcggagcaagcggcgcgttccagaacctcgggcaagaccagcctctcccagagcatccccaccgcgaagcgcaaccttctccagagcatcccccagcggagcgcacccttccccagagcatccccgccgccaagcgcaaccttccagaagcagagagcggcgacatggccaagaaaacagcgatcggcatcgacctgggcaccacctactcgtgcgtgggcgtgttccagcacggcaaggtggagatcatcgccaacgaccagggcaaccgcacgacccccagctacgtggccttcaccgacaccgagcggctcatcggggacgccgccaagaaccaggtggcgctgaacccgcagaacaccgtgttcgacgcgaagcggctgatcggccgcaagttcggcgacccggtggtgcagtcggacatgaagcactggcccttccaggtggtgaacgacggcgacaagcccaaggtgcaggtgaactacaagggcgagaaccggtcgttctacccggaggagatctcgtccatggtgctgaccaagatgaaggagatcgccgaggcgtacctgggccacccggtgaccaacgcggtgatcaccgtgcccgcctacttcaacgactcgcagcggcaggccaccaaggacgcgggcgtgatcgcgggtctgaacgtgctgcggatcatcaacgagcccacggcggccgccatcgcctacgggctggaccggaccggcaagggcgagcgcaacgtgctcatcttcgacctggggggcggcacgttcgacgtgtccatcctgacgatcgacgacggcatcttcgaggtgaaggccacggcgggcgacacgcacctgggcggggaggacttcgacaaccggctggtgagccacttcgtggaggagttcaagaggaagcacaagaaggacatcagccagaacaagcgcgcggtgcggcgactgcgcacggcgtgcgagagggccaagaggacgctgtcgtccagcacccaggccagcctggagatcgactctctgttcgagggcatcgacttctacacgtccatcacgcgggcgcggttcgaggagctgtgctcggacctgttccgcggcacgctggagcccgtggagaaggccctgcgcgacgccaagctggacaaggcgcagatccacgacctggtgctggtgggcggctcgacgcgcatccccaaggtgcagaagctgctgcaggacttcttcaacgggcgcgacctgaacaagagcatcaatccggacgaggcggtggcctacggggcggcggtgcaggcggccatcctgatgggggacaagtcggagaacgtgcaggacctgctgctgctggacgtggcgccgctgtcgctgggtctggagaccgcgggcggcgtgatgacggcgctcatcaagcgcaactccaccatccccaccaagcagacgcagaccttcaccacctactcggacaaccagcccggggtgctgatccaggtgtacgagggcgagagggccatgacgcgcgacaacaacctgctggggcgcttcgagttgagcggcatcccgccggctcccaggggcgtgccccagatcgaggtgaccttcgacatcgacgccaacggcatcctgaacgtcacggccactgacaagagcaccggcaaggccaacaagatcaccatcaccaacgacaagggccgcctgagcaaggaggagatcgagcgcatggtgcaggaggccgagcgctacaaggcggaggacgaggtgcagcgcgagagggtggctgccaagaatgcgctcgagtcctatgccttcaacatgaagagcgccgtggaggacgagggtctcaagggcaagatcagcgaggctgacaagaagaaggtgctggacaagtgccaggaggtcatctcctggctggactctaacacgctggctgagaaagaggagttcgtgcacaagcgggaggagctggagcgggtgtgcaacccgatcatcagcgggctgtatcagggtgcgggtgctcccggggctgggggcttcggggcccaggcgcccaagggaggctctgggtcggggcccaccatcgaggaggtggattagaggctctttctggcgctccaggtgtgatctaggagacagatgggtggccttgaggactttgggttattgtcgtttaggacattaactccttcgttcggtctgcaatcaagtcctaggtttaagcaaactgccttccatttactctgtggaatttcacgtgtgctttgcattcccagtaaattagtactgggagtgtgtctttgcaatagatataatttcctgccttcaagtcagcactgcccccccccccgaagttatttcttttgcaggacagtcagagctatattgatatagcaagaggtgtgttacaaaaacaccaggacactgttgagttcctttgtgtttggactctcccctgggcgacagtgttgaggcactgttaagtcaggagctcaggggccaccggtggatcactgaaagctgagactctgttgcttctcccgtttgacactctgttgctttccttgcatggtggctcacctaaggctgagactcttgttctccttccctgtataatcttgcctggccgttgcacttgttccccagtgtgtgaactcggagatgagtttacaccaccactgttagttcacgttttttgtttttacataaccatcctgaactcaggtcaatttttactggctatttgaaaataaacttcaaaagaacttgccaggtcttgtgtctgttgtcttttgaggtcaggaattgctgtgtatgtcttacagattggactacgcttgactgacattttatggtaagatggggtggggagcacccagggactttgcattgggatctgcttgtgtgagaaaaatgaaagcagtcagagcaaataacaaccagtccggggctgaggtctagtacagtgcaga

SEQ ID NO: 36 - DNA, 2445 bp, Homo sapiens
  misc_feature (1)..(2445): cDNA for heat shock 70kDa protein 1A
ataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgattggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattaggggcctttccaagattgctgtttttgttttggagcttcaagactttgcatttcctagtatttctgtttgtcagttctcaatttcctgtgtttgcaatgttgaaattttttggtgaagtactgaacttgctttttttccggtttctacatgcagagatgaatttatactgccatcttacgactatttcttctttttaatacacttaactcaggccattttttaagttggttacttcaaagtaaataaactttaaaattcaaaaaaaaaaaaaaaaaa

SEQ ID NO: 37 - DNA, 352 bp, Rattus norvegicus
  misc_feature (1)..(352): cDNA for chemokine (C-X-C motif) ligand 7
caccatgggcttcagactcagacctacatcctcctgcaccagggccagctctcttcgtaacctacaggtgctgctgagcctgcttctcgttgcgctggttccccttgcagttggaaaacctgataacaacgagccatttatcgaattgcgctgcagatgtacgaatacactctctggaatcccactgaactctatctcccgtgtgaatgtgttccggccaggagcgcactgtgacaatgtggaagtgatagccacactgaagaatggaaaggaagtgtgcctggacccgactgcccctatgatcaagaaaatcgtcaagaagatattggaaggttattgaccagctgcttcg

SEQ ID NO: 38 - DNA, 1307 bp, Homo sapiens
  misc_feature (1)..(1307): cDNA for chemokine (C-X-C motif) ligand 7
acttatctgcagacttgtaggcagcaactcaccctcactcagaggtcttctggttctggaaacaactctagctcagccttctccaccatgagcctcagacttgataccaccccttcctgtaacagtgcgagaccacttcatgccttgcaggtgctgctgcttctgtcattgctgctgactgctctggcttcctccaccaaaggacaaactaagagaaacttggcgaaaggcaaagaggaaagtctagacagtgacttgtatgctgaactccgctgcatgtgtataaagacaacctctggaattcatcccaaaaacatccaaagtttggaagtgatcgggaaaggaacccattgcaaccaagtcgaagtgatagccacactgaaggatgggaggaaaatctgcctggacccagatgctcccagaatcaagaaaattgtacagaaaaaattggcaggtgatgaatctgctgattaatttgttctgtttctgccaaacttctttaactcccaggaagggtagaattttgaaaccttgattttctagagttctcatttattcaggatacctattcttactgtattaaaatttggatatgtgtttcattctgtctcaaaaatcacattttattctgagaaggttggttaaaagatggcagaaagaagatgaaaataaataagcctggtttcaaccctctaattcttgcctaaacattggactgtactttgcatttttttctttaaaaatttctattctaacacaacttggttgatttttcctggtctactttatggttattagacatactcatgggtattattagatttcataatggtcaatgataataggaattacatggagcccaacagagaatatttgctcaatacatttttgttaatatatttaggaacttaatggagtctctcagtgtcttagtcctaggatgtcttatttaaaatactccctgaaagtttattctgatgtttattttagccatcaaacactaaaataataaattggtgaatatgaatcttataaactgtggttagctggtttaaagtgaatatatttgccactagtagaacaaaaatagatgatgaaaatgaattaacatatctacatagttataattctatcattagaatgagccttataaataagtacaatataggacttcaaccttactagactcctaattctaaattctacttttttcatcaacagaactttcattcattttttaaaccctaaaacttatacccacactattcttacaaaaatattcacatgaaataaaaatttgctattga

SEQ ID NO: 39 - DNA, 2850 bp, Rattus norvegicus
  misc_feature (1)..(2850): cDNA for chemokine (C-X-C motif) ligand 12
ccgcactttcactctccgtccacctcggtgtcctcttgctgcccagctctgcagcctccggcgcgccctcccgcccacgccatggacgccaaggtcgtcgccgtgctggccctggtgctggccgcgctctgcatcagtgacggtaagccagtcagcctgagctacagatgcccctgccgattctttgagagccatgtcgccagagccaacgtcaaacatctgaaaatcctcaacactccaaactgtgcccttcagattgttgcaaggctgaaaagcaacaacagacaagtgtgcattgacccgaaattaaagtggatccaagagtacctggacaaagccttaaacaagaggctcaagatgtgagaggtgtgagtcagacgcctgaggaacttacagaaggagcctaggtctgaagtcagtgttagggaagggcccatagccacttcctctgctcctgagcagggctgaagccatttccaagggacttgctttgcagtttgctacactttcaccatttgattatatagcaagatacatggtaattattttattttcatttagtctgattctccaatgtcattggtgacaggccaaggccactatgtcatctcctttgttctagtatctttcccatgaaggaccttttctgaatagtggctcccaaggtttgtctctttgagctgaggcaggaggctcacctttttctgattagaaactgggtgttcctacccccaaggattgcagggctttccccaagctgaggcaggagtgtgaggtcagggaagagcgagatccaccctcatcccatgctctcctcttcatcccaccatgctcatctctgtctcatccatcaccgtgtgtctgcaagactgtctccatgaccctgaaaaaggactctctcaagatgaactcctttactcaaatgggacagcaagaaggaaaaggaagtgtctgttgttccgcccaaacccttcgcgcgtctatgtcttgtttggaatattgtctcttcaaccccctgcttctgttgacctccatgaccaatgtctcatctgtgcactgtctctaacccaaatgcaaaggctgtgtatgaggtaatggccctgaggtccaggttttcatggaaacagcgcactgtctccctgttcacaggctcattttggacacacaaagcccaaagaaaggtggtttgcaacagagctcagctctaagaactgtagatccttcatattttcgtaactgttagctctaaattgtgggttcttagcttcctggaaccgaatgcattcttctattgaggactagcacgtctcagttctttccaattatttttaaaagccaatgaataaaagcatcagcattgggccccactgggcgggcatttctctagaaaggggagaaccacctacctttccttaggacagccgaccagcacggcccaggaagtgttttgtcttctgcagtttttatacaagctccctgccacctttgacaaacgcacagttaagagtcagtatctagttcttcagagacaaagatggagggagtaagaaggggaagggaaacggagaaagctaccaaaagatcatcctcaaaagcaggtgtttgagagtgaacgagctgtagaattgttagtgatgtgtgtgtggtgagggatttctataaatagtcattcaagttgatttcacagcagatgaaaaatccaaccagcaagattttgatcaaatttggacaacagcaacaaaactaaaaatgtgaagccagttgggataaggggcatatggtttgctgcagactgggtccccatgtggattcagaattattttaaactctcttgacatccggggcccccacaagagaaatctggattgctgtgcaatggccacttagcatctaatccaagctttgaaggaaacaaatacagccttgcaccttctctccagttagggatccttttaaagtctccttcacagggaggataaagagactgggtagaaactggagggagatgaatttgtgtatcaattccgctgctgacagtcagtttctagatggagacagcctgcctagagcaaatgtgcatttaaataggacatttacatgggaaaagcctctccccaccttaatcccccatgctcccgctttcaaaatacaagcacagcagtccttgaatggttgttgaccccggacacctaactgtccctgatggtcctggtgctgcccgaattcccttgggcgccaagtaacctgccaggcagccagtccctctgtcaccagcctttgcatctggatagggaaaagggggttggagacatacagtctgctttgtgttgaaacccagatttgtaccctgtgtttacactgtgctgcctgctctcggggacagtgggaaggaagtgcagccgaggtggcagacccctctgattcattgcctggtcggctttgagggagggtttggagagcaaaaggctgcattcctctgtgggacttggcctgaggcctttaggcctctccatcgagttctgtttatcttctcatgggtgattatctcggcggcgtcaccaggggcttcctcacagaagtcattcctccgcagagcttgcagtgtctacgcagcgatggtttcagtgttgcatgtggtgaatactgtattttgtttcagttctgtctcccagataatgtgaaaatggtccaggagaaggcagcttcctatacgcagtgtgtgctttcttattctcgtttttaatatatgacagttatttgagaggccatttctactttgaagtcatatcaatgaaaaatgatgtatcttcacctacaatttttcctaataaagttctgtattcgaatgta

SEQ ID NO: 40 - DNA, 3545 bp, Homo sapiens
  misc_feature (1)..(3545): cDNA for chemokine (C-X-C motif) ligand 12
gccgcactttcactctccgtcagccgcattgcccgctcggcgtccggcccccgacccgcgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtttggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttccccttccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcttgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtttgtgctgtggtgtgtccccctctgtccaggcactgagataccagcgaggaggctccagagggcactctgcttgttattagagattacctcctgagaaaaaaggttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaattgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgtagccaccaa

SEQ ID NO: 41 - DNA, 4927 bp, Rattus norvegicus
  misc_feature (1)..(4927): cDNA for exportin 5
ggtcccgccaggggaaccgtgcgtacctcgtgctcctgggacgccggtggttttcccggcactagccatggagatggagcaagtgaacgcgctgtgcgaggagctagtgaaagcggtgacggtcatgatggaccccagctctacccagcgctaccggctggaagccctcaagttttgtgaagagtttaaagaaaagtgccctatctgtgtcccatgtggcttgaagttggccgaaaaaacacaaattgccatcgtcagacattttggccttcagatcctggaacacgttgtcaagtttcggtggaacagcatgtctcgattggagaaggtctacctgaagaacagtgtcatggagctgattgcaaatggaacactgagcattttggaggaggagaaccatattaaagatgttctgtctagaattgtggtggagatgatcaagcgagagtggcctcagcactggcctgacatgctcatggagttggacactctctccaggcaaggggaaacacagagggaattggtgatgttcatccttttgcgactggctgaggatgtagtgacctttcagacactgcccactcaaagaagaagggatattcagcaaacgttaacacagaacatggaaagaatcttgaattttctactcagtacacttcaggaaaatgtaaacaagtaccagcaaatgaagacagatgcctctcaggaagcagaggctcaagccaactgtcgagtgagcatcgcggcactgaacacgctagcaggctacattgactgggtgtctttgaatcacattactgctgagaactgtaagctggtggaaactctgtgcctgcttctcaatgagcaggagctgcagttgggagccgctgagtgtcttctcattgcagtcagcagaaaaggcaagctggaggaccggaagcgcttgatgatcttgtttggggacgtggccatgcactatgttctctccgccgcacagactgctgatggaggaggcttggtggaaaaacactacctcttcctgaagagactttgtcaggtgttgtgtgctctgggaaatctgctgtgtgcactgctggccttagatgctaacatacaaacaccggtaaactttggaatgtacctggagtcttttcttgctttcacaacccacccaagtcagtttctacgctcttcaacccatatgacgtggggagccctcttcaggcatgaagttctgtcccgggatccgtcgctgttagcggtgataccaaaatatcttcgtgcttctatgacgaacttggtcaagatgggttttccttccaaaacagacagccccagctgtgaatattcccggtttgactttgacagtgatgaggacttcaatgctttcttcaactcctcccgagcccagcatggagaggtggtgaggtgcgtgtgtcgactggatcctaagactagcttccagatggcggcagagtggctcaagtatcagctgtcagctgctattgacactggacctgtgaactcatgttctacagctggaactggagagggaggcttctgctccatcttctcaccttcatacgtgcagtgggaagccatgactttttttttggaaagtgtgatcaaccagatgtttcgaacactagagaaagaagagctcccggtaaatgaggggatagagctgctgcagctggttttgaactttgagatgaaggatcccctcatcctgtcctgtgtcctcaccaatgtctcagcactctttccatttgtcacatacaagcctgccttcttgccccaggtcttctcgaagctcttttcatttgtcacttttgaatctgttggggaaagtaaggcccccaggactcgggcagtcaggaatgtaaggagacatgcgtgttcctccatcaacaagatgtgtcgagactacccagaccttgttctgcccaattttgacatgctttatagccatgtgaagcaactcctctccaatgagctcctcctgacccagatggagaagtgtgctctcatggaagccctggttctcgttagcaaccagtttaaggattatgagcggcagaagttattcctagaggagctgatggcaccggtggtcaacatctggctctctgaagaaatgtgcagagcactgtcagatattgacgccttcattgcctatgtgggtgctgatctgaagagctgtgacccggctgcgcaggacccgtgtggcttgaaccgtgcacgaatgagcttttgtgtgtacagcatcctgggggttatgagacgaactacctggccttccgacctagaagaagccaaagctggggggttcgtggtgggttatacacccagtgggaatccagtcttccgtaacccctgcacagagcagatcctcagacttctcgacaatttgcttgccctcgtaagaactcacaatactttatatacaccggagatgctaaccaaaatggcagagcctttcaccaaggctctggatattgttgaatctgaaaaaacggcaatattaggattacctcagcctctcttggaaatcaatgaccaccctgtctatagaaccattttggaaagaatgcagcggttcttcggcatcctctatgaaaactgttaccacatcctagggaaggcaggcccttccatgcaacaagatttctacactgtggaggaccttgctacccagctccttggttcagctttcgtcaacttgaacaatattcctgacttccggctcagatctatgcttcgtgtcttcgtgaagcccctcgtgctcttctgccccccagaacactatgagaccctgatatctcccatccttggaccgctcttcacctaccttcacatgaggctttctcagaagtggcatgtcatcaaccagaggagtgtcctgtgtggagaagatgagattgcagaggacaaccccgaatctcaagaaatgctggaggaacagctggtgaggatgttaacccgagaagtcatggacctaatcatggcttgttgtgtgtcaaagaagactgccgagcacactgccactcccactgcggatggagacgacgaggagatgatggccaccgaagtagccccttcgtctgttgtggagctcacagacctgggcaaatgcctgatgaagcatgaggatgtttgcacggcactgttgatcacagcatttaattccctgacctggaaggataccctgtcttgccagagggcgaccacacagctctgttggcctctcctcaaacaggtgatgtctgggaccctgctcgcagatgccgtcacgtggctcttcaccagtgtgctgaaagggctgcagatgcatgggcaacacgacgggtgcatggcttccctggtccacttggccttccagatatacgaggcactgcgccccaggtacctagagataagagcagtaatggagcagatccctgaaataaacaaggagtctctggaccaatttgactgcaagcttctaaacccctcccttcaaaaagcagctgataaacgacggaaggaccacttcaaacgcctcatcgctggctgcattgggaaacccttgggagaacagttccgaaaagaagtccacattaagaaccttccctggcttttcaaaaaatccaaaccaatgctggagacagaagtgctggacagtgaggagggtggactggccaccatctttgaaccctgaatcaagcttttgggcgtccttctccggcctcagttgtcatgtcttcttccctgtgtagctatctctaggccttcctcacactgccacctcacttcccaccgagtcagcctggaggagctcagatccaggtctcctgtgagagggcctgagtcatcccactaaagtaaaagggtattgggacctccaccattccaagaaggtgacctgggagtcggcagccacctgcagtatgggcttctccaagaaaagctagagcaccatggctttgcccagagcagctgcaccagtgcagacaggaggaccaaccagggtgccgtgcccagccccactgacctgatcgtccggtttcctttggacggcacttgagccccttgccatggggctttcgggatcctcctccactccaggggcatgtctggccgaggtgctacatgctgacctgtgtctggccagcttcctcgtctcaacctaaagctagaatctggtcgcttggtgggacccgtggtacaggctatttctaaagtctgagcacaccatccagggacaagagctgggcagaattccttagagtggacatcttgcccttctgacggaccctggagggttccccagtgacacaccaaccatacaagagtaatcagaccaaggaggcttctgacgagcgagcagagccactgtccctcctgggactagctgagtccgggtgcctttgagaatctcaatggtcttttaagcttctgaaggtcaaaaagtgttccagtcattgccaacacttttggtccctggaaagggaatgaagcaaaccaagatgggtgtgggacctagttctgcctattgtgacaggcagatgtgacctgtgtgtgtgtatgctaacaggcccactgtccacccagccccaactttgacctggtgtgggacacaggcaggcatggcagagttggagtcagagctaggaggtagctactagggttcgaggtcactgctgcacaaagccggccgtcctccctctgcgtggtgcttggggtctacagattgtaaggtgagctcagtatttccctccagacttgatagctgcctactcctgtctgcttagggctacggaggatgtagttgtatttattatgttgtaatatttttaacatcctatgacttcatgctagagattttctattgtttatagaacttttgtagaaatgctaactctaaagtacatgtgcatgtcagtaaaacctgtttcatacagaa

SEQ ID NO: 42 - DNA, 5378 bp, Homo sapiens
  misc_feature (1)..(5378): cDNA for exportin 5
acttccgcgggcgcgtgcgcagtggcgggccgggcgcgtgcgcagcgccggcttcttcccagcgcgagttgctgctaacgctcctcctccagcttcccacccccgccgcccgccgcggcccggtaccaacgggtggtctcgccgagggagctgtcgtgcctcgtggtcccgggactgcagtggtgtgcgctctcggcgcgtggcgctaggcatggcgatggatcaagtaaacgcgctgtgcgagcagctggtgaaagcggtgacggtcatgatggaccccaactccacccagcgctaccggctggaagccctcaagttttgtgaggagtttaaagaaaagtgtcctatctgtgtcccctgtggcttgaggttggctgagaaaacacaagttgccatcgtcagacattttggccttcagatcctggaacacgttgtcaagtttcggtggaacggcatgtctcgattggagaaggtgtatctgaagaacagtgtcatggagctgattgcaaatggaacattgaacattttggaagaggagaaccatattaaagatgctctgtctcgaattgtagtggaaatgatcaagcgagagtggccacagcattggcctgacatgctaatagaattggacactctttccaaacaaggggaaacacagacagaattggtgatgtttatccttttgcgactggcagaggatgtagtgacttttcagacacttccccctcaaagaagaagggacatccagcaaacattaacccagaacatggaaaggatcttcagttttctgcttaacacacttcaagaaaatgtaaacaagtatcagcaagtgaagacagatacttctcaggagtcaaaggcgcaagcaaactgtcgagtaggagttgcagcactgaatactctagcaggctatattgactgggtgtctatgagtcacatcactgctgaaaactgtaaactcctggagatactgtgtttgctgttgaatgaacaggaacttcagttgggagccgctgagtgtcttctcattgcagtcagcagaaaaggcaagttggaagaccggaagcccttgatggtcttatttggagatgttgccatgcattatatactctccgccgcacagactgctgatggaggaggtttggtagaaaaacactacgtctttctgaagaggctctgtcaggtgttgtgtgcgctgggcaatcagctgtgtgcattgctgggtgcagattctgatgtagaaacaccatcaaactttggaaaatacctggaatcttttcttgctttcacaacccatccaagtcagtttctacgctcttcaactcagatgacttggggagccctcttcaggcatgaaatcctgtcccgtgatcctttgctattagcaataataccaaaatatcttcgtgcttccatgactaacttggtcaagatgggctttccttctaaaacagacagccctagctgtgaatattctcggtttgattttgatagcgatgaggacttcaatgctttcttcaactcctcccgagcacaacaaggagaggtgatgaggttggcatgtcgtttggatcccaaaactagcttccagatggctggggagtggctaaagtatcaactatcaacttttcttgatgctggttctgtgaattcttgttctgcagttggaactggagaaggaagcctctgttccgtcttctcaccttcattcgtgcagtgggaagccatgactctttttttggaaagtgttatcacccagatgtttcgaacactaaatagagaagaaattcctgttaatgatggaatagagctattgcagatggttctgaactttgataccaaggatcccctcatcctgtcctgcgtccttactaatgtctctgcactctttccatttgtcacctacagaccagagttcctgccccaggtcttctctaagctattttcatctgtcacttttgaaactgttgaagaaagtaaggcccccagaacccgggcagtgaggaatgtgaggaggcatgcttgttcctccatcatcaagatgtgtcgtgactacccccagcttgtgctgcccaattttgacatgctttataaccatgtgaagcaactcctctccaatgagctactcctgacacaaatggagaagtgtgccctcatggaagccctggttctcattagcaaccaatttaagaactacgagcgtcagaaggtgttcctagaggagctgatggcaccagtggccagcatctggctttctcaagacatgcacagagtgctgtcagatgttgatgctttcattgcgtatgtgggtacagatcagaagagctgtgacccaggcctggaggatccgtgtggcttaaaccgtgcacgaatgagcttttgtgtatacagcattctgggtgtggtgaaacgaacttgctggcccactgacctagaagaggccaaagctgggggatttgtggtgggttatacatccagtggaaatccaatcttccgtaacccctgcacagagcagattctgaaacttcttgacaatttgcttgcgcttataagaacccacaatacattatatgcaccagaaatgctagccaaaatggcagagcctttcaccaaggctctggatatgcttgacgcggaaaaatctgctatattaggattacctcaacctctcttggaactcaatgactctcctgtcttcaaaaccgtcttggaaagaatgcagcgtttcttctctaccctctatgaaaactgttttcatatcctagggaaggcaggcccttccatgcagcaagacttctatactgtggaggaccttgctacccagcttctcagctcagcctttgtcaacttgaacaatattcctgactaccgactcagacccatgcttcgtgtctttgtaaagcctctggtgctcttctgtcccccagagcactatgaagccctggtatcccccatcctcggacctcttttcacctacctccatatgaggctttctcagaaatggcaagttatcaaccaaaggagcctgctgtgtggagaagatgaggctgcagatgaaaacccagagtctcaagagatgctggaggagcaactggtgaggatgttaacccgagaagtcatggacctaatcacggtttgctgtgtttcaaagaagggtgctgaccacagtagtgctcccccagcagatggagacgatgaagaaatgatggccacagaggtcaccccctcagctatggcagagcttacagacctgggcaaatgtctgatgaagcatgaggatgtttgtacagcgctattaattacagccttcaattccctggcctggaaagatactctgtcctgccagaggacaacctcacagctctgctggcctctcctcaaacaagtgctgtcagggacactgctcgcagatgcagttacgtggcttttcaccagtgtgctgaaaggcttacagatgcacgggcagcacgacgggtgcatggcttccctggtccatctggccttccagatatacgaggcactgcgccccaggtacctggagataagagctgtaatggagcaaatccctgaaatacagaaggactcactggaccagtttgactgcaagcttttaaacccctccctgcagaaagtggctgacaagcgccgaaaggaccaattcaaacgcctcattgctggttgcattgggaaacccttgggagagcagttccgaaaagaagttcacattaagaatcttccctcacttttcaaaaaaacaaagccaatgctggagacggaggtgctggacaatgatgggggtggcctggccaccatctttgaaccctgaatcaagcttttgggcatccttcctcggcctttcttgtcatctcttctttccctttgtagccgatctctaggcccttcttgcactgccacctcactttccaccactgtcagcctggaaagagatccaggtctggagctggagagaacaggccctgtgcaggaccagaagtaattatactaaagtatcaagaaagggagttagggcttaaactattctgtctagatgtcccagatagttcccattctacttggagatttggcttttccaagaaaagctagagcagagcagcccttctcccacaagccctcccacccccgtgcagccacatacctgtacagaatggtaactaagggtgctgtgcccaaccctgcgactagcaaggctcgcagcaagagcacagccctcaactacttgtgccagagtttctcttggaccactccaactcccactgagcccttttgctgctgggctggcaggaaactttccccactccctaaggggcatgtctgggttaggtgctaagtgctgaagagaacttggtcagttctctcaactttgctttgggcaagaatctggtcacctgatgggatccatggtacaggctactgctaaacttggcacagtatcaagtatagtacctccaaggaccagggctgggaagtctttagtgctaacatcccctttagagttcacacatcttgcccttccatgaatgacccctcagtctggcctccccagcctcaaggtccactcaggcacaagagccacagtaccctagatagtgtcacatgacaccgttgtcatccaaggataatacagaccaactaggctacatctgtgatgagcagctagcaaagccgctggtcttctcctaggactaagtccaggtgccttccacaatctcatggtctttcaggtccctggttacttttctcaaaggccatttccaaaagaatacatgccttcacatcacaacctgtactgtgagtccattctagaggtcactgaaaggccctgtaaagagaggacatggatacgggacctggccctgaggttattactggccgtaaggcagagttaatccatacagaaaccagtgtgtccatgtgctctgcacaaaaacagacctgttgtccatccagtccactgacaagagggtttccccgagagccgaagtggactgaagctacagtttttagctggtgcgggccacaggcagggtcagattgagaagaagcaaagctggggaagcagaagttgggagtcttgtgttgctccctcttcctgtgtggtgctctgggtttctgtggatcgtgaaggcgatctcaagagtgtttccctccaaacctgatagctgcctattcctgtctggttggggctgtggaggatgtagttgtatttattgcattgtaatatttttaacatcctgtgacttcatgctagaaattttctattgtttatagaaactttttgtagaaacattaactctaaagcacatctgcatgtcagtaaaaatctcagtttcgtacagaaagggaaaaaaaaaaaaaaa

SEQ ID NO: 43 - DNA, 3573 bp, Rattus norvegicus
  misc_feature (1)..(3573): cDNA for carcinoembryonic antigen-related cell adhesion molecule 1
agccgtgttggagccaaatctcttccccagagaagaacctagcaggcagcagagactatggagctagcctcggctcgtctcctcagagggcagattccctggaggggactactgctcacagcctcacttttaacctactggagccctctcaccactgcccaagtcaccgtagacgctgtgccacccaacgttgttgaggagaagagcgttcttttactcgctcacaatctgccgcaggagttccaagtcttttactggtacaagggaaccactttgaatccagacagtgaaattgcacgatatataagatccgataatatgagtaaaacaggacctgcatacagcggcagagagacaatatacagcaatggatccttgttcttccaaaatgtcaacaagactgacgagagagcctacacactatctgtgtttgatcaacaatttaatcctatacaaacgtctgtgcaatttcgtgtataccccgcattacaaaagcccaacgtcacaggtaacaactccaatcccatggagggtgagcccttcgtatcattaatgtgtgaaccgtacactaacaataccagctacctgtggagcagaaatggtgaaagcctctcagaaggtgacagggtgacgttctctgagggcaacaggactctcactttactcaacgtcaggaggactgacaaaggatactatgagtgtgaagcccggaacccagcgactttcaatcgaagtgacccattcaacctggacgttatctatggtccggacgccccagttatatcccctccagatatttatttgcatcaagggtcaaacctcaacctctcctgccatgcagactctaaccctcccgcacagtacttttggcttatcaatgagaagctccagacatcctcccaagagctctttatctccaacatcactactaacaatagcggaacctatgcctgcttcgtcaataacactgtcactggcctcagtcgcaccacagtcaagaacattacagtctttgagccagtgactcagccctccatccaaatcaccaacaccacagtcaaagaactaggctctgtgaccctgacctgcttctcaaaggacactggagtctccgtccgatggcttttcaatagccagagtctgcagctcacagacaggatgacgctctcccaggacaacagcaccctcagaatagaccctattaagagagaagatgccggggactatcagtgtgagatctccaacccggtcagtttcaggataagtcacccaatcaagctggacgtaatacctgatccaacacaaggaaattctggcctctcagagggtgccattgcaggcattgtgattggatctgtggctggagtggccctaatagcagcgctggcatacttcctttattccaggaagactggcgggggaagtgaccatcgagatctcacagagcacaaaccctcaacctccagccacaatctgggtccttctgacgactctcctaacaaggtggatgacgtctcatactctgtcctgaacttcaatgcccagcaatccaaacgaccaacttcagcctcttcaagccccacagaaacagtttattcggtagtcaaaaagaagtgacattgtctgtcctgctgactgcaccagtgatgcattcccagattctctccctcacgggagagtccctgcacacacgcgcacgtgcgcgtgcacacacacacacacacacacacacatgcacacacacacgcacacacgcacgcacacacacacatgcacacacgcacacacacatgtatacactctattgactccttccgtgttaatagaagaaaaggtgtatcctggagcctacagaaaacccaaccctttctaggcattaaaatttggccgagagagaggaaccaaaacggcccaccccttcacttttctgttttaaattgatgtattctgagcacattcactcctccacccaactccaagtcctgccctgtcagagtctgacttgaatttcgataaacctcggaggtcacctaagtgcttatgccaaaaaccacaaaaccaaacaaagcaaaacagaagcaggaaatggccgatcccatgtcttaaggctgatgcgaagccaccacacatgaagatcaaagccataggcattcgttcgctgggcagtccgcacaccagccaactgtcacatttactgtgacattagagtctctgaaccatgggtcttcacgcctgagcccttctccaggaaacccactcagagcaactagagaaaccccttggcagcaaacaccagcaattgctaagcagggtgcagaagaaaagacgacatttctcacatctaccaggatggcgcccaggaaagcagggctggcggttttggactgagagacatatggaatttgaaaagggaacagagagataccctttgaggagtgtcgaaattattttaaaagaagccatatttggaggcgttggaattttcctcttcctaagacaatccactatttgacatttgtagctgctgactttcctttcgctgggcgagctgatcagtttgccttagggcaactaaatctcacgtctcccttggccccccgcacgcaagccctttggatcgtgagctccaaaccaaaaataaataagtgaatcgtgaggcggtcccttgcagaaccactgtcatgggtcaggctcctacttttctgtattagtcaagaaaagcctgagttttcctttctcgtttttatcctgtcgttctggccacagcccggacagtcttcctctgagatctctagcttgtctaccttcttgtgtttcttttaaactctcacgaaactgcttaacttcaaaaagtaaaataataactttcctcatctacaccagaaagagacccaactccgtttctggaaacctaggatcagcctccagctgccctctgaactttctgccttgtttcccctttcttttgactctaatacctcatgcagtgtcctacctgttaggatgacagcacaggccactaggcctggccacccgtacattctgtctgccctgcactctcaggaaaccttggcctctgtctgtttggttcatgtgaagtgtgcccttaaaggaacagtcactcataccggaggcattgtttacagcctggaacattctacacagtgcatcactggaaatgggctgctagcagggttctcgtacccagccatggcctttctgtagcctcggaaacctgcctttccaccagggatgagtcccagcagcggagaaaaggcaaatgtagtctcaggaacccgctgttcaaaggcacaccctctgtgcctgttaaccaaaggttgtatttgatatcgagtatttggtctcatgccatgcctaacaggcttctgagagaattgtttagttctcccactcagggatttctttcagggatttccagccctaacatgacctgtgtatattttgcttcacccacgcaatgctgggttacttcattttgcccggctaaagccaaatcggagttatttgtaaatttgctctatgttcataataaaaatttatatatcactgata

SEQ ID NO: 44 - DNA, 3528 bp, Homo sapiens
  misc_feature (1)..(3528): cDNA for carcinoembryonic antigen-related cell adhesion molecule 1
aaagctctgggccccagggaggaggctcagcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcgaaaaaaaaaa

SEQ ID NO: 45 - DNA, 551 bp, Rattus norvegicus
  misc_feature (1)..(551): cDNA for family with sequence similarity 183, member B
tttctcttggcaaggaaagggtcaggtagcttgacttctccgagcaagagaatgaccatggcaggacgtttggggcagaagatgaatcaggatgaagttcaccagaatcagatcatgcgggaactatttctcaaagagttccggacacagaagctgtacacgcagtatcacgtgaatcccctgcgcaaggttcacacaatcactcggaagcccatgtcttggcatgataacttggaggagccagaagatgctaagttcctgaacctcgttcaccatgctgcacagggtccaaaaaagaaatattcagagacacagaccgaagcccaagaaattggatgggacccacatcccttgatcaacccagatcgtcaggaccgcaggttgaaccacttcagagtctaccatgacatcactctgtacaaagctaaactgtggagcttgggagaagacgatcaccagaagtaatgttctcttggtggcatggacctgcacactgagttcccaatgcatgtgttgcccaaagaaataaatgccacttctgttaaacatta

SEQ ID NO: 46 - DNA, 1647 bp, Homo sapiens
  misc_feature (1)..(1647): cDNA for sequence similarity 183, member B
gtgatggaatgtcctgaggggataaaagctggagtcggtctcagcacatctcagttactcattttgactcgtttggacaagtgagttactacagcgctgatttggaaaaatgactagaaagctacctccaatttctcggcctcaaccccagcctcattttctttctgttccttcccgctctcgtcttccagccctccttccccccgttactcctcaaacttcctgccgtcagcctcctctccagagccgtccaagttgttcgattttctgccctacccgtttgcccaagctgactaatgtcgccccttcccagtaccctacctcaaggaacttcctgcccggcccttcctctgttactaccagggcgtccgtgtgccgccgccctgttcgtcaggcacccctttcccggtgtccccctatctttccccctcaccctcctcatagcccttctttctagtcccctgtactctagtccccactccctatccagccacccccaaccagacctgacggcttgcaaatctcaccctggagaaatggtttccctcctactggggcagttcccaggcggtccagaattgccaggacttgtggattcccagcagcgacctggcagacacggggaatccaagaccatggcaagcgaacattcggcctggcctgcgcctttcctctcctgcctgggcagccagactgcacaagcctctgcatttgaactggcttctccgtggaacttggtttgtaaccacggagcaaactgcaggaccaggggcagagaagttgatggcgagcctttttcctggcagtatccagaggcggttctagtaaagaggctggattccgagaggccagagcggtatcatacgaacgccgttgcctggagacggagtggggtgccattgcctagagactgcagaaggcccgcagccaagcgagtggtaagaggacgccgagagagccccggacccacggagcagccccaaggctatggcgggacacccgaaagagagggtggtcacagatgaggtccatcagaaccagatcttgcgggagctgtacctcaaagagttacgaacccagaaactccacacgcagtaccatgtgaatcccctgcgcaaggttcacaggattacgaggaagcctatgtcttggcatgataacctggaggaacccgcagacgccaggtttctgaatctcattcaccatgctgcccaggggccaacgaagaagtacccggaggcacagactgaaaaccaggaaattgggtgggactcagaagccttggtcgacccagaacgccgtgaccacaggatgaaccacttcagggtctacagtgacatcactctgtacaaagctaaaatgtggagcttgggagaagatgatcgccacaagtagcatctcagctgtggagtcaggccctggatttaatgccctaaatatccactgcctagaagactaaacattattttaaccccccgtcccccatccataattcatggataatggcaaaaattaggaagcataaaaaatatgcggaagaaggaaataaaaattgcccattatctcaccatatggaagtgactaatgttagcattttaaaccatttgtttttaaaattaataataaattgcatatatttattgtgta

SEQ ID NO: 47 - DNA, 3690 bp, Rattus norvegicus
  misc_feature (1)..(3690): cDNA for solute carrier family 24 member 3
atgccgccgccgggcgaccaggaccgcgcgcgccgccgcagccgccgccggcggagggaccttctgctgagccagctctgcttcctggcttcggtggcgctgctgctctggtcgctgtcgagcctgcgagagcagaaggaacttgacctcatggatctcatcggggaagatagaaaatggatggtggggaggaagctgatgcaggtgaatgatacactgacttctgaagatgcaggactccggagcagcaagaactgcacagagccagccctgcatgaattccccagggacatcttctccaatgaagaccgacgacaaggagcggtggtactccacgtgctctgtgccatgtatatgttctatgcactggccatcgtgtgcgatgacttcttcgtcccatccttggaaaagatctgtgagcgcctccacctcagtgaagatgtggcaggagccaccttcatggcagcaggcagctcggccccggagctcttcacatcggtcataggagtcttcatcacaaagggggatgtgggagtcggcaccatcgtgggctctgctgtgttcaacatcctgtgcatcattggcgtctgcgggctctttgcaggacaggtcgtggctctttcttcctggtgcctgctgagggattccatctactacacactgtctgtggtcgcactcattgtgttcatttatgatgaaaaggtttcttggtgggaatctctagtccttgtgctgatgtacctcatctatatcatcatcatgaaatacaatgcgtgcatacaccagtgcttcgaaaggaggacgaaaggtgctgggaacatggtcaacggactggccaataacgccgaaatcgatgacagcagcaactgcgatgccactgtggtgctacttaagaaagccaatttccaccgcaaagcgtcagtgatcatggtggatgagctgctgtctgcctacccacaccagctctctttctctgaggctggccttcgaataatgatcacaagccactttcctcccaagacccggctctccatggccagtcgcatgttaatcaatgagagacaaagactgataaacagcagggcatataccaatggggaatctgaagtggccatcaaaatcccaattaagcacactgtggagaatggaacagggcccagcagtgccccagacaggggtgtgaatggaacccggagggacgacatagttgctgaggctgacaatgagacagagaacgagaatgaagacgagaacaatgagaatgacgaggaggaggatgaagatgatgatgaaggcccctacacgccattcgaccccccctctggcaaactggaaactgtgaaatgggcattcacctggcccctgagttttgtcttgtacttcactgtccccaactgtaacaagccccactgggagaagtggtttatggtgacgtttgcttcctctacactgtggattgcagccttctcctacatgatggtgtggatggttaccatcatcggctacaccctagggatccctgatgtcatcatggggatcaccttcctggctgccggaaccagcgtgcccgactgcatggccagcctcattgtagccagacaagggatgggggacatggctgtgtccaattccattgggagcaacgtgtttgacatcttgattgggcttggccttccctgggctctgcagactctggctgtggattatggatcctatattcggctgaacagcagagggctcatctactccgtgggcttgctcctggcctctgtctttgtcacggtgtttggcgttcacctgaacaagtggcagctggacaagaagctgggctgtggatgcctcttcctgtatggcgtgttcctctgcttctccatcatgacggagttcaacgtgttcacctttgtgaacttgcccatgtgtggggactaatgattgaccaggtcacctgccgaggcccaggtccttcttttctgtgcaatacgagacccggccgcacccgagtcatgcggcccctcaacggccgcggcgtccgtctctcccgtgctgtctgcgggcctgtcctggtggtctaggctctccctgccacctcctttgctcccaaactcccttggtcctacctcccagccagcccccctcacccacccaccccattcctgtgtcttcttccgtgtgaagacgtccaccatccacatgaattttcaagctccatatttgaacggtgactgagattctagaacaaaactggctgctaactggcccaagccaggcacaacttactccaatccctcctcctttttttaagttatttcacagaagataccctaatttatgaccagagactttgaggaaacaggagagggtccgctgatggcaggaagtattgggtttgttttgtttgggagtgattcctaggttaccgggtttgggaaatggtttaatttgagggggttcattgtttctttacttttctttcagagacagaggactcccctcgagtcttctctcactgggctctggcttaatagatggtccacagattgtactgtctcctgggggagaatccaggcacgttgggaatttaataattagttgggaaaccactgagcgagaccaccaggaatagatgcatgacatgacatttttctgctcaaggacacatcactaaagtcctgaaggcttcacctgggggtggggggtgtaaggtgttagaggaagaaacctgcttctcacatgttcactatgcccatctccctcccaactaggttctttctagtttttcaagcaatagttctcgcctgcctaaggcctttgacaggaagaccctgactaactgtaaggcaccaggcagtcaacgacagaaacttccccagtgtgtgagtccttgacatgtgctcttgttgcttggcatttgtggtgacagggagtgaccccagaggctatcactgtgctctggacagaagttacagacactggtttcttggaaaatgaagagaagcacattttgcacagatgtcgtcaatgaagtcccaatttgccacttggtatcgagtacactggaccctgacagctggctcttgagcaaacatccttcctcacaggatgccaccaataagctagccagtttcatccctcccgtcccagagggatggccagggagggaggaagcagagaactgctgcattgaaaaccacagaatgggttcacctcagactggctcaagggcacaagttattgtgaacatttcgccaatcactgctcaaccgccctgctagatttttctatgatgctgaattattatgcagaataactccaccaagttgagatacacccatgcttgttacacttgtatttattgaaactgtggattcttgcccgcccgtgctctcccttgtatttactttaagcactgatcacttatcatccactcggtatggttttcccagtcccttgtacacatcctggtttgaattgtaaaaaataacctgctacagactggttgaacgtttctgtctgagctgcaagccaaggggtggcgtcaaccgaggaccaggagtggaaatcacccatttgggctctcagagctaagacacacttactgatttggttgcacattttgcactggtttatggcgattgttttcttggacgaatggtgtaaaataaacttctctgttctgtatcctccctcaaa

SEQ ID NO: 48 - DNA, 3905 bp, Homo sapiens
  misc_feature (1)..(3905): cDNA for solute carrier family 24 member 3
gcgacgaggagggcgacgacgaggagacgggcagcggcgaggaggaggaagaggaggcggaggcggcggccgggtgggagcgcagcgaggacgcgcggctgctgcgcgcagggctgcctcctgccgctgtccccgccgcggccgcccgcgacaggagcggccgccgcccgccgaggccgccgcccggccgcccgaggatgcggccgtccggcgacgaggaccgcgcgcgtcgccgccgccgccgccgccgccggagggaccttctgctgagccagctctgcttcctggcctcggtggcgctgctgctctggtcgctgtcgagcctgcgagagcagaaggagcttgacctcatggacctcgtaggggaagacagaaagtggatgatggcgaggaagctgatgcaggtgaacgacactctgacttccgaagatgccggactccggaacagcaagaactgcaccgaaccagccctgcatgaattccccaatgacatcttcacaaacgaggatagaagacaaggtgcggtggtcctccatgtgctctgtgccatatacatgttctatgcgctggccattgtgtgtgatgacttcttcgtcccttccttggaaaagatctgtgagcgcctgcacctcagtgaagatgtggctggggccacattcatggcagcgggaagttcggccccagagctgttcacatcggtcataggggtcttcatcaccaaaggcgatgtgggagttggcaccatcgtgggctcagcggtattcaacatcctgtgcatcattggtgtctgtgggctctttgctgggcaggttgtggctctttcctcctggtgcctgctgagggattctatttactacacgctgtctgtgatcgcgctcatcgtgtttatttatgatgaaaaagtttcctggtgggagtctttagtccttgtgctgatgtatcttatctacattgtcatcatgaaatataacgcttgcatacatcagtgctttgagaggaggacaaaaggtgccgggaacatggtcaacggattggccaacaatgctgaaattgatgacagcagcaactgcgacgcaactgtggtgctacttaagaaagcaaatttccaccgcaaagcatcagtgatcatggtagacgagctgctgtcagcctacccacaccagctttccttctctgaggctggccttcgaatcatgataaccagccactttccccccaagacccggctctccatggccagtcgcatgttgatcaatgagagacaaagattgataaacagcagggcttataccaacggggaatctgaggtggccatcaaaatcccaattaagcacaccgtggagaatgggacagggcccagcagtgccccagacaggggcgtgaatgggacacggagggacgatgttgtggctgaggctggcaacgaaacagagaatgaaaatgaggacaatgagaatgatgaggaggaagaggaggacgaggatgatgatgaaggaccgtacacaccattcgacaccccctcgggtaaactggaaacagtgaaatgggcgttcacctggccgctgagtttcgtcttatacttcactgtacccaactgcaacaagccgcgctgggagaaatggttcatggtgacgtttgcttcctccacgctgtggatcgcagccttctcctacatgatggtgtggatggtcacaatcattggttacaccctggggattcctgacgtcatcatggggatcaccttcctggctgctgggaccagcgtgcctgactgcatggccagcctcattgtggccagacaagggatgggggacatggctgtgtccaactccattgggagcaacgtgtttgacatcctgattggcctcggtctcccctgggctctgcagaccctggctgtggattacggatcctacatccggctgaatagcagggggctgatctactccgtaggcttgctcctggcctctgtttttgtcacggtgttcggcgtccacctgaacaagtggcagctggacaagaagctgggctgtgggtgcctcctcctgtatggtgtgttcctgtgcttctccatcatgactgagttcaacgtgttcacctttgtgaacctgcccatgtgcggggaccactgagccgccgggtgcccacagaggctcagctccttcttttctgtgcaatacgagacccggccgcaccccgagtcacacaggcccccggggccacggcgttcgtctctcctgtgctgtcctcaggcctccgctcctgttttggtggcccaggctctcccctgacccatcctcgctcccccacctccttgggtcatgcccacccaccctttcctgcctcctccgtgtgaagacatccaacatccacgtgacttttccagctccatttttgaacagtgactgagattctagaaaaactggctgctaactggcctgagccaggcaacactgattccaatccctcctccttttttaagttatttgatggaagactcacctaatttgtgacctgagactgttgaagaaatagagaggagggggcccgttgattacagagagcatttgggattttgtttggtttggagatgatgcctaggttactgggtttggggggattgttttcttttgggggccttccccttttactccttttcttccagagatcaagagcttctcttgcatcttcttccactgggctctggattaatcaattacccaaaggctgcacctgccgtgttgtctgggcttgcatcccagatgtgttggagtatgcatggatgtagtgctttttagaggagccactgggcaaggccaccaagaacaaatgcatgacattttatagccaaggacgcctcgctaaagtcttatgggcgtcccctggggttgggggggcacaaggttttggaggaagaagacaacttccctcattccatcatcaccatctctttctcactaggttctttctagttttcaagcaatagttctagcctgccttggacaagggggccccagttaaacaaactacccatccatgagctgccaggcagtcaaaaacagaagcttccccgacttgtgagtccctgagatgtgctcttgttgtttggcatttggggtgacagggagtgacccagaggccaccactgcttttcatgcaggagttacagacactggtttcttggaaaatggagagaagcgcactttgcacagacgtcgtcaattaagtcccaatttgccacttggtattgagtacactggaccctgaccactggctcttgggcaaacgtccttcctcacggggcgcctccgccaagccggcccagctgcacccctcccttcctggagggatggccagggaaggagaaaacagagaactgacacctttgaaaccacagaatgtgttacatgcagactcgctcaagggcataagttattgtgaacgtttttgccaatcactgctcaacagccctgctagattttgtatgatgctgaattattatgcagactaattccacccagttgagacacaccatgcttgttcacttgtatttattgaaactgtggattcttgcccgtgctgtcccttgtatttactttaagcactgatcacttatcattcattcggtatggttttccctgtcccttgtacacattctggtatgaatttgtaaaaataacctgctacaaattggttgaatgtttctgtctgtggtgcgaaccagcattaacggatggggcacgtgcccaactgaggaacaggagaagaaatcaccaatttgggctctcagagctaagacacacttattgattctgttgcacattttgcactggtttatggcgattgttttcttggacggatagtgtaaaataaacttctctgttctctaaaaaaaaaaaaaaaaa

SEQ ID NO: 49 - DNA, 3522 bp, Rattus norvegicus
  misc_feature (1)..(3522): cDNA for potassium voltage-gated channel subunit beta-1
cttttgtccgaggatgagtggggaggggtcttgagaagcctccacctccgcatcctccgaagttccggagggaggtagttgacaggcagagtggatggagaccacgggtccgcggggccctgcttgctgttagctgcttcttgggatgctcctctccagttgagcctcctgcctgtgatccagagggtgctggcagcgcgtcttgcggtatgcctgcgttgctttgagccagcgcccagaaaccagccctctgcattcaggcacctcggactggctccaggcagggctgtgtgcagtcaccatgaatgctcagctctaggcttctgcaccagcctcctgaccacatggatctggctgtttaatcagccgagatcacaacctgggaagattggggacgctgatatgcgctccagctgctgagacaacttccagcttggctgtttacttcttcgggtcccagggactctgtcctacacctcagcctctcctgccactgtgtgtgggttctgaagtcagctgctgggactcctccgaaggcatccaggaaggggggcaagaggggttaaaagacagccgaaatcccctccaactgctcaccctctgccgaccgcctgtgctgtggggttctgagaggaccttgcgctgtccggggaagcaatgcaagtctccatagcctgcacagagcacaatttgaagagtcgaaatggtgaggaccgacttctgagcaagcagagctccaccgcccccaatgtggtgaacgcagcccgggccaaattccgcactgtcgctatcatcgctcgcagcctggggaccttcacccctcagcatcacatttctctcaaagagtccactgcaaagcagactggcatgaaatataggaatcttgggaaatcaggactcagagtttcgtgcttgggtcttggaacatgggtgacattcggaggtcaaatttcagatgaggttgctgaacggctgatgacaattgcctatgagagtggagttaatctcttcgacactgcagaagtctatgctgctgggaaggctgaggtgattctggggagcatcatcaagaagaaaggctggaggaggtcaagcttggtcatcacaaccaaactctactggggtggaaaagccgaaacagaaagaggattgtcaagaaagcacatcattgaaggactgaaaggctccctccagaggctgcaactggaatatgtggacgtggtctttgcaaatcgcccagacagcaacacccccatggaagaaatcgttcgagccatgacgcacgtgatcaaccaaggcatggcaatgtactggggcacctcgaggtggagcgcgatggagatcatggaagcctactctgtcgcgcggcagttcaacatgatcccgcctgtctgtgagcaagctgagtatcatcttttccagagagaaaaggtggaggtccagctgccagagctctaccataaaataggggttggtgcaatgacatggtctccacttgcttgtggaattatttcaggaaaatatggaaatggggtgccagaaagttctagagcttcactgaagtgctaccagtggttgaaggaaaggattgtaagtgaagaagggagaaaacagcaaaacaagctgaaagacctctctccaattgctgaacgcctgggatgcacactacctcagctggctgtggcatggtgcctgagaaacgagggtgtgagctctgtgctgctgggatcatccactcctgaacaactcattgaaaaccttggtgccattcaggtcctccctaagatgacatcacatgtggtaaatgagattgataacatattgcgcaacaagccctacagcaaaaaggactatagatcctaaggcaatgcatgagccacttaagctgcatgagtaaatcctggtgggtaggcggtgcagaacggccttaccagccactcttttcaatcacttagcagcttgctgctcaacctctagtgtcccaggattctctgaggtctctctgctgttgctaccactgtgcaccctgaattctgaacacatctgaaaattcacaagccagaaaatccattctattttcttatcttggactggtcccctctcctggcattggtctgtgtacctcatgtttttgcaatggaaagaatattggggtaggggaatctattatcttcagaaggctgtacatatatatttttttcaaaagaacaaaattcacaagatacagtgtatgacctgcataggaaacaggatagcctcactctttagtcttgcttgggagaacaaacagaattaatttactttttttttaatatttcatattcacactagcaagttttatgtcatctgtaattcaaataaaacttctaggtatttactttcactaccttgaaagttatgttgttgcctggccctgtacaatttaagtggggtgtctctcagggttctaacatatccatttacatagagttataaaggcatgaccaccccaatctttactagatacacaatgctaatttataaaagggttatagtgctatggaaacagctttaaataaaatgttgtgttcatttttttactttctattaaaacctgcatatacgttcaattgcttgtaggcacaatttctgttaagtttaaatgtttgagctttaaaaatacatacatgctcagttttgtaattaaacctataaatacccagaaagggaagtgtcagtttaccactgggacattattatacaatgcttggtattttctgaagagttattaacatgaaaacaaatcatagacaatgtaaaaagaaaaatgtttttctcattcctcagcacatgctagatgaattaacttgcccaaagaaaaagcaaaatattcaagaagctcctgattctaaatgtaccacacactacaccgtatgcatgaaagttctttgcatggaataaaggggcttagctttgcactcagaatctgagatatacttagccctgcctctacaggttaattcccctcactatcccacttgagaagaactgtaagactatactgtgtgggtatctgtaaataaggataaaacacaaagggtttttgttgttgttttgttttctgtaaaggttgggtcattttctaactctatttgtctatctatagatgaacaacatcctacttcatctagttagcaagttcttcatcactttaagatttacacaagaaatatatgaacttaaaataattttcagtcatgtaccacataaccatgttgtacatcttaaactatcagtgaaagataccaagaatcatgattgttactacctagactccagtaggcaacttggaagtttcacccatgagtatctgattaatgatggagctgaattaactttccaactattcattgtaaatttaatccactgaactgtatgtataaatgagtggagtgctgtctaaatgaaaatcaattcatttgcttagagtaataaaggcatctgcaatctcataaaaaaaaaaaaaaaaaaaaaaaaaaaagg

SEQ ID NO: 50 - DNA, 3835 bp, Homo sapiens
  misc_feature (1)..(3835): cDNA for potassium voltage-gated channel subunit beta 1
atgcacaggcctgggcagggacacacaaccaacagctctgtcgggaacttaggagcctgctcgcctacaaaaatgataagagagatctaaagaaaacaagctaaaccttttgagggacatactgaagccagataacccaaggtattcacagcaagatacagtgagtcttaaagttaagcaccgtgcaattagctttgcttccttgggtttttgaaacatgcatctgtataaacctgcctgtgcagacatcccgagccccaagctgggtctgccaaaatccagtgaatcggctctaaaatgtagatggcacctagcagtgaccaagactcagcctcaggcggcctgcaaacctgtgaggcccagtggagcagccgaacagaaatatgtggaaaagtttctacgtgttcatggaatttcgttgcaggaaaccaccagagcagagacgggcatggcatacaggaatcttggaaaatcaggactcagagtttcttgcttgggtcttggaacatgggtgacatttggaggtcaaatttcagatgaggttgctgaacggctgatgaccatcgcctatgaaagtggtgttaacctctttgatactgccgaagtctatgctgctggaaaggctgaagtgattctggggagcatcatcaagaagaaaggctggaggaggtccagtctggtcataacaaccaaactctactggggtggaaaagctgaaacagaaagagggctgtcaagaaagcatattattgaaggattgaagggctccctccagaggctgcagctcgagtatgtggatgtggtctttgcaaatcgaccggacagtaacactcccatggaagaaattgtccgagccatgacacatgtgataaaccaaggcatggcgatgtactggggcacctcgagatggagtgctatggagatcatggaagcctattctgtagcaagacagttcaatatgatcccaccggtctgtgaacaagctgagtaccatcttttccagagagagaaagtggaggtccagctgccagagctctaccacaaaataggtgttggcgcaatgacatggtctccacttgcctgtggaatcatctcaggaaaatacggaaacggggtgcctgaaagttccagggcttcactgaagtgctaccagtggttgaaagaaagaattgtaagtgaagaagggagaaaacagcaaaacaagctaaaagacctttccccaattgcggagcgtctgggatgcacactacctcagctagctgttgcgtggtgcctgagaaatgaaggtgtgagttctgtgctcctgggatcatccactcctgaacaactcattgaaaaccttggtgccattcaggttctcccaaagatgacatcacatgtggtaaatgagattgataacatactgcgcaacaagccctacagcaagaaggactatagatcataaggcaatgcatgaaccacagaagctgcatggttaaaatagcggcctgtgcccagtacagaaaggtgttactaaccagtcttttgaatcacttagcagcttgctgctcaacctctagtgtccctccctggattctttgaggtgtctgctgtcgctaccactgtgcacatctgaaaactcacaaccaagaaaatccattctattttcttatcttggactggagtcacctattcttgcattgctgtatacacctcatgcttatgcaatgggaagaatatgggggccagggggtgtggtactaccttcaggcatttggtaactcaaagaaggctgtacagatatattttttcaaaagaacaaaatccacagatgcaatgtgagttgcgtaagaaacagagtagatagactaaattcagtgaaggaaaggaattgagagatttttcttagtaaatagattattgttaagtaaatagttattaaaaatatatctcactgcaaaaaaaaaaaagcagtatcttcactcaaaagtcttgcttggaagaataagcagaaataattttatatattttttttctattttcacattcatactaacaagttttgttccatttgttattcaataaaacaaaaatttctaggtatttgctttattacctttcaaatatttactgttgcttggccccaagaatggccttgtacaacttatccagaatgtctattaggattctaatgttatgtccacttacaagtagagacagtaaaaggatgaatacccaatctttagtgacaatgcagctgatttatgaaagagagggctacactgctatggaaacttagcttcaaagaaaatgcaatgtatctgcaattaggtgttcattttttactacattttattaaaacctgctttatactttcaactgcttgtaggcacaacttctgcaagtttaaatatttgagctttacaaataaacatacacatgctcagttttttaagtaaacctgtaaaatacccaggaaggcaaatgttcattgtttaattagcactgggattttataatataatgtttggtatttttgaggcattgttaacatgaaagtcaaccactggctttgtgaaaaatgctatgtcactattcagaatatgctgggtaaattgacttgcctagtgaaaagcaaaatgttaaagaaagaacttctggttctataatcatattatatgcactaaactatatgcatgaaagttctttgcatggattaatggggcttacccttgttgcactcgaaatctgaggtgtatctagccctgccactattggctacttaccctcattaatatcccacttgagaaaaattgtgagactatactgtgtcaatatctgtaaaaagagagaaaacatgttttgtttttttttgaagggggtggtgtgggagtggccctttaactctatttggctatctgaggatgtacaaaattctcatttaattttctggtcagcaagttccccacacagaaatcactctgaggtttacagaagaactgtaatattattttaaaatgcgatttttctgtcattagttctagatatgtacttcatggttaaattctaaatctgaaaatgctagtgggagatatcaagaaattttctttttgattactagtacctgtattctaacagagagtttgaattttttgcccgtgttatcagaatgatggaaattgatcattttcagttgttcattgtgtattcaatccagcgaactgctgtatgtatagaggagctgaggtgctgtctaatgggaaatgtgatttgattgatttatttgcttagagtaataaaagcattttgtgcattcaatcttacaatagattttatctgatctttcactgtaaggcttatgtgtattttccatatatgtaaaacatacacatgtaaaatatatgtggccataaattaggcaataatcatctaaataaaaattatattggcagctaagctgcactgaatactcaacagaaatagtagtgatcaaaagtctgagatttaaattgcaatccaaacatattctatcatcaatcaatcaatggagattaactactgaagataccatttctcactgggttttagaacactccgcactaaggttaaggcttcacaaattcacttttaaaaacttacttgcaaatcaaaaccaaatataaatattatggcttagctgtgggaacagccagttttttatatgcgaatctgtcatggtaaatatactaatgtatttcacagttagttagttagttagttatgtccttaagaaaaaattccacgcactcttattattttcttttctcttccaaaaagaattccctggcagagaatctatcattccaaaaggtaacct

SEQ ID NO: 51 - DNA, 3397 bp, Rattus norvegicus
  misc_feature (1)..(3397): cDNA for phosphodiesterase 5A, cGMP-specific
gcagacttgagagtggagcggagggacgagagggtggagcggagtgcgtggcgggcgggtcccccgaagctcgaggagtcgccgcgaagcccgcgcgcgcgcttgcctccggcgcgccgatctgggctgaactaacaagctctgctttggagaaggggctggagccccgccaccggcgccttggggcaacggccgtcgagcagctggtgctggccgcccgagccgggaggtcgactctcgccgggcggggatggtggacgagccggcggggactcgcccgcgcgcggccgccacagggcccggcgctgcccgccagggggcgacgccacgcggggtccgcagccggcgacccgcagcgaagaggcggcgggggccataagggagccggtcgggcacccacggaactccgcgggcccccagagtctccccagtccaccatccgaggagttcggaaacgagcgccccacctctgctatgttgccctttggagacaaaacgagagacatggtcaatgcatggttttcagagagagttcacaacattcctgtgtgcaaggaaggcatccgggcccacaccgagtcctgctcttgctctctgcctcaaagtcctcatgcagataataccacccctggagcaccagccagaaaaatctctgcctctgaatttgaccgacctcttagacccattgttgtcaaggattctgagggaaccgtgagctttctctctgactcaggaaaaaaggaacagatgccactaacctctccaagatttgatagtgatgaaggggaccagtgctcaagactcttggaactagtaaaagatatttccagtcatttggatgtcacagccttgtgtcacaaaattttcttgcatatccatggacttatctctgctgatcgctattccctgttccttgtctgtgaggacagctctaaagacaaatttctcgtcagtcgcctctttgatgttgctgaaggttcaacactggaggaagcttcaaataactgtatccgtttagaatggaacaaaggcattgtgggacatgtggcagcttttggagagcccttgaacatcaaagatgcctatgaggatccccggttcaatgcagaagttgaccaaattacaggctacaagacacaaagtatcctttgtatgccaattaagaaccacagggaagaggtcgttggtgtagctcaggccatcaacaagaaatcaggaaacggtgggacattcactgaaaaagatgaaaaggactttgctgcctatctggcattttgtggcattgttcttcacaacgcccagctctatgaaacgtcactgctggagaacaaaagaaatcaggtattacttgaccttgctagcttaattttcgaggagcagcagtcattggaagtaattctgaagaaaatagccgccactattatctccttcatgcaggtgcagaagtgcaccatcttcatcgtggatgaagactgccctgactcgttctctcgtgtgtttcaaatggagtgggaggaagtagggaaatcctccgagcctttgacaagggagcacgatgcaaacaaaatcaattacatgtatgctcagtatgtcaaaaacacgatggagccacttaatatcccagatgtcaccaaggacaacagatttccctggacaaatgaaaacatgggccatataaacacgcactgcatcagaagtttgctttgcacgcctataaaaaatgggaagaagaacaaagtcataggggtttgccaacttgttaataagatggaggagaagactggcaagattaaggctttcaaccaaaacgatgaacaattcctggaggcttttgtcatcttctgtggcttggggatccagaacacacagatgtacgaagcggtggagagagccatggccaagcagatggtgacgttagaggtcctgtcttatcacgcatcagcggcggaggaagaaacaagggagctccaggctctagcggctgctgtggtaccgtctgcccaaacccttaaaattaccgacttcagcttcagtgacttcgagctgtctgatctggaaacagcgctgtgtacaattcggatgttcactgacctcaaccttgtgcagaacttccagatgaaacacgaggttctttgccgatggattttgagtgtcaagaagaattatcggaagaacgttgcctaccacaattggaggcacgcctttaacacggcacagtgcatgtttgctgctctgaaagcaggcaagattcagaacaagttgacggatctggagacgcttgcattgctgattgccgctttaagccatgatttggaccatcgtggtgtgaacaactcatacatacagcggagcgagcaccccctggcccagctgtactgccactccaccatggagcaccatcacttcgaccagtgcttgatggttctaaacagcccaggcaaccagatcctcagtggcctctccattgaagaatataagaccacattgaaaataatcaagcaagcaattttagccactgacctagcactgtacattaagagacgaggagaattttttgaacttataagaaaaaatgaattcagttttgaagatcctcttcaaaaggagttgtttctggcaatgctgatgactgcctgtgatctctccgccattactaagccctggcctattcaacaacggatagcagaactcgtggcagccgaattctttgatcaaggagacagagagagaaaagaacttaacatggagcccgctgatctaatgaacagggagaagaaaaataaaatcccaagtatgcaagttgggttcatagacgccatctgcttgcagctgtatgaggccctgacccacgtctcagaagactgtttgcccctgctggatggctgcaggaagaacaggcagaaatggcaagccctcgcagaccagcaggagaagacgctccttaatggggagagcggccaggccaagcgggactgaccttctccccctcctcccctcccccacccccaccgggtgttcgaactgccctaggcctggtttcgccatacactatgcagactcgagtgcactttgccactgctgtattttatttttgcacaacttctgagagcatgaggtgtttcagagattgctacctactttccgtgcttttgttttatgccactgaagtggagccgtcagtcatgcccactctaacactttgataagagtggtgtaaactgcattcccacacgcagcttggttatgcttggggagtatgaataaccggcctttgtgtttctgagtcaccagtctttcaaacacgttaggaatcgttctcggaagtgggttaggggcgaactgaatgtgtcctgggacatttaaagccatcctgaaattaacgctgagtggatgttag

SEQ ID NO: 52 - DNA, 7005 bp, Homo sapiens
  misc_feature (1)..(7005): cDNA for phosphodiesterase 5A, cGMP-specific
ggcctggcgctccggccgctttgtcgaaagccggcccgactggagcaggacgaagggggagggtctcgaggccgagtcctgttcttctgagggacggaccccagctggggtggaaaagcagtaccagagagcctccgaggcgcgcggtgccaaccatggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttggggattattttaaataattggtttttgtgttttctgaattaccattttttcaagaatgtttggaatctttcctttttcaaaagtaggttaggagcaaattatcatacattctgtgacatttaaagcctttataggatagtgaaaaatgctggctgagtggattttaagagaaataattgtatttgttaacagtgtctttttttaaaaagttaaggcactctgaaacaaatggaaagtcctatgaaactgtattgtaaagaaaacattatttaattgatatgctgttttgtgagagaacaggcaagacagaactttgtcacttcagtgcagtacatttttctgaaagctacccataaaatcactttcatctcacctacctgatgcaaagcaggtgaaaccttaggagatgatccagtcactgacttgattgagggataagtgtgatttagaaatggaatggccttggatgtctatcagtgaagaaaaatgttctgttagaagatctctctaagagtttttttccttctgagcttccttttcaaaataaaagtgacaattgtagcattgacttgaagtgagacatggttatagataagagagtacaaaatgactctttttcctgtcaattgaaatttaaagaaaagttttaattatataaatagcaaagggctattgccaatactagggtcaaaaatgaatttgagggaacagtgggtaagaaactttatgcctgaataacatttagcagtattgtgattgaaaaattgccatattttgatgtataggacaagtcaactgagatccagagaatcctggatgtgaatgctaaacactggcccttaactcacattcaatgtattttcttcccataacatttagtatagttaatattttcttagaatttgagcccatttaagtggattaatattctacatgtgtgcccctaaagacacatttactcaatattggagaagtagataatgaattaagcaactggtctaggaaaggaaaatttgtttcaaatatgcaggaatgtttggatttggggagagtagaaggagagatttgcttgatttgttaacttctacctccaacccacaaaaaagatatttgatctgagtttctatcactaatttggatagaaaatttctaagggacatggtaatccagcattctcaaggacctttcgccaaaatgtgttttccatctatgtcccgattcccctaaattttgcctaaaattcagtatgttccttaagtttttaaaattctgagtgtgtacaaatatcttgacataatgcagttttatttttatcattctggtaaaaaaacaaaaaatagaagcaaaacacattgtattgccattattttgtatttggtaaaggttaatctaggaagttaccaactgtttaatgctatatgtattgtatacttgtattttcaggatattttattttttttgccatacagataaaatttgtaaggttgcccctttgtggcactggtgtgtaaaatacacagactatcactaaaataatagttatatatacatacaggtgtatacttatgcatgcatacataaatccttagtatagaaaaattgcataaagaatagcaatctttaataaacctttttattacattgtgatttagcagttatgctaaaatatgtacttatgctttagtagtttgtttggtcccctctagtatgtgtcactgagaaattttttaaagacatggtagatcgtgtttagaggctttgtatgtgtgtcattttaataagcaagaagatatatttagattagaaatggtttggtctgcctttgaatattgtttattttactttactagttgagacattaaaggaagctgggcaatgcctattttatttctttgttggatattttagttcatacaaagcagagtacttctttagggctggttaattggttcaaataatttttaatttcctttctagtatcttctcaagttggaaaaatatacatacagtcctccttcaccttactctgtatttatattacccataactagcaagaagttcttgttctagattttttgtttgtttagttataacagagtaacataccatttaattacaatttttagccagaaaagtccccactattttactaacttgttaaaagatatctatataattgcctggccttatatttttcagtagattagaccgtgccaatcacaatcctgggtggatttgtgtaagtcacttaacctctgtgtgcctaaacaagttgtgcttttttaaaaggagttatgtttgggcaaagcctttgtcttcaagcagaatgtcacagaaggcagctactttataagccccaatgggccatggagaccactgtcagaaatgggatattagtctagagagaaggtgatctattcccacatgtcatttctaatgttgagtttccatgactgaacaaagagaatatatttattcagcttcacttgcagatcactagtgaatgtgagatttagagctcattgagtatattgcttcaaggtacaaacccaggatgatgatgttgtcaccactgtctcttaattttgaataatagtttcctttaataggagtattagagataagaaagtatatgaaaatatactggaaatattggattcttggagaaaactgttcagtcacagatatattcttgcctagcagtgaagtgcctttattttcagcatagcaaataaatattagacctgttccaatttgatctacaatttttttctgtgtttttcaccagattgtactcctaaaacttaacaggccatcacaagcaattgtcttttgtttacaagattgatttaatatgagaggatacaaaatgtcatcgttatcctctcttatgaacaactgtagtcaaaataaggtggcacaatttaattgttttgtatcagaaatacactgacccaccttttattgagtcctgccacatgttaggtaccgtgctctgctgtggagacagagcagtgaccccaaggagctcacggtccctgaaggaggtgctagagaagagacttagcttctgatactgccaatttaatgtgagaacatggggtatactgcatcatttccattttcatcaataacatatgttttatgcaccttctttacctgaaacttactaagaatctaccagtaaacaaacatcctgtctttttgcaagtatgaatcacttaacctgctgatagttgaagaacactttaggagttttgtattcttgtatatagtttattttttccatgtgctagccaggtaaagattacacagttcttctggactgttaaattgtgcatggttttggaccccttctgctctactacagagagtgaagaagaaagtattaaagctcactttaccattccatatacttactaaaagcctgtgtaaacatgtcttaatgaatgttgttgaaagcaatgtaaatagttgaaaatataaatttatattacagtttaagaaaaccttatgaggcatcactagccactgttaatatctatttgtattcttataccttttcaatatatttgaacaaatatagtttctggcactatttttatactaggaaaaggagttactatgtatattatgcttagcttttaaggcattttaaataaccatgaatgttgatttcattactttcctttcctccatcacgagagtcatttcagatgactctttcatgacaaaatcactttaaaggaacacttacctccgattcctgtataaagtcatgagatggtcaaggtggttttccattgtgcaaattcttcacctgtcagtggtttcctcattttgccatgctttgtaaaaataaaaagaatgatcaagtaggtatgaaaaaaaaaaaaaaaaaa

SEQ ID NO: 53 - DNA, 3559 bp, Rattus norvegicus
  misc_feature (1)..(3559): cDNA for Rho GTPase activating protein 28
atggaggtggaggactcgggcggcgtggtgctgaccgcctaccactcgcacgcgcgctcgcaaccgcagggcgcggagccacgctgcgcgtcccgggccggccatccgctcagcagaaaatccattcctcgctgccggagaatcaacaggatgctctccaatgaatccctccatccccccacctttagtcgctccaactctcaagcctctgtcgacagcagcacatctgtggaggagttctggaaggaaatctcgagtatcaaagagagcagcatagggggacaggaggagcagtcgcccactgcccccactgaggtcaaacctgtggatgaaggagaacttgaagcagagtggctacaagatgtgggattgtcgaccctgatctccggtgacgaagaggaagacggtaaagccctcctgtccacgttgactcggacacaagcagctgcagtgaagaagagatacaacacgtacacccagactctgaggaaaaagaacaagcaagccatcagggatgtcagagacgtctttggagtcagcgaatctcctcctagtgattcttgtgagcacgctactcagttggacagtaccaaggaagagaaggatctgccaggcgttaccaagacaagcagacctctgccagacgatgcttctctcaatagtactaccctatccaacggtgcccaggatgaagaaggtggttttgtggccctccagagtggttccgtgtcaatacttgaggctatcccagatattcccgttcataccaatggatctgcagacactgagcagtcggttcagcgtgggctgagtgatgatgattatctagagaagaacattccaccagaggctgaagagctatcctttgaggtgtcttactcagaagtggtgacagagaccccagacagaaataaatggaagaagtcagacattaagaaagaagactatgtcttacctaaatttattgttcagaaaacgagatttgggttgaccgagacgggggacctgtctgccgaagacatgaagaaaatccgccatctttctctgattgagctgacggccttcttcgatgctttccggatacaactgaagagaaacaaaacagagagagtgagaggccgagacaacgggattttcggagtgccacttacagtcctcctggacaatgaccggaagaaggaccctgccgtgaaagttccccttgtgttacaaaaattttttcagaaagtggaggaatcaggtctggaatcggaaggaatctttcgactgtctggatgtactgccaaagtcaagcagtaccgtgaagaactggatgccaggttcaacactgataagttcaaatgggacaaaatgtgtcacagagaagccgcggtcatgttgaaagcattcttcagagaactgcccacctctctcttccccattgaatatatccctgccttcatctctctgatggaaagaggacctgacatcaaagtgcagtttcaagccttacacctcatggtcatggccctgcctgatgccaacagggacacagcccaggctctgatgacattcttcaataaagtgatcgccaatgaatccaagaaccgcatgagcctgtggaacatttccacggtgatggcgcccaacctgttcttcagcagaagcaagcactccgactgtgaggaactgctggtggccaacactgcagcccacatcatccgcctgatgctgacgtaccagaaaattctgtggaaggtcccatctttcttgatcacccaagtcagaaggatgaatgaagccaccatgctgttgaagaagcagctcccaagtatgaggaagctactcaggagaaagaccctagagcgggaggtttcaaaccccaagacctcaaaggtaccacaaaaatcaccttcctcaagaagaatgtctgatgtgccagaaggtgtcataagggtccacgctccacttctgtccaaagtgtccatggccattcagctcaacagtcagaccaaagccaaagatatcctggcgaagtttcagtatgagaacagccacgcttcatctgaccgtattaagatgcagaaccaaaggttatacgaagttggaggaaatataggacaacactgcttagaccctgatgcatatatattggatgtgtatcatgtaaatcctcatgcagaatgggtcatcaagccctagacacgcctttaaaatatgcccaggatggttcctgccttgtattatatgccaagaagatcccacatttgtgggggagagcactgccctgaagtatttgctcctgtaaccctggttctcctggaagggtgctgttgccagctcctgccatacgaaccacagagtgaggagctggtccaccctgagctcaaccctcctgtcacagtttcccataagaaggtggggcccaaaggaaaggccagcgctactcaccgaggagctatctgtgtagcagaaatagacttggccctgcagctgggaagaggctccacttctgatgtgaaacatacaatatttatgctctgacttgctcctgaattggaggagggccatcagtgtaaagacggctgaggatcatttcatactgcacagcacactctgagaacgtgctcgcactgtggtgcaaaggaaatttccccaagccctgagggtccagatgctactttatgactccatgtttcaccggtactcattgccaagccccgtttggcaaaggccatcttgtgcatcgagagcttccctgtggtccacagccatggtgtctttctttttccctctcagttatggtgctccttgaataaattgcctttcacgagtcagcatggatttcctttatagatcaaatgcattgtcttttttttttaacatttcacagctctagccacatctgcagcccccaaaccaaaaaatacagaagtgtgtttacccataagcactgaaaaatgccgctagacctagaatttataagttgcaaaaattccttgtgctttttgatcattagccagagatgcgaccaagcactgtggattggccctgtgctgtggaagctggagaccaccttatgtccgcatcttgtacacccaatgtctagatgtgcagtgggagacattcagtcctcgtggttctaacaggaacaaatccaaaatgtcccgagggacaggacagctccgcctttcatctcagagattggagttagagtcctcattcatccaagaccatgggcctcatcttggactccacaggctgaacactgatggacttaacagcggttacaacatttaagactggtgcggctgtcggtcagttcgaacaaagctgagttacaaacccagaactgttcccttacctacataccctgtgtccggatgtaatgcaaggacactgaacgtgcatgtgcagagatctatgctgtgctctgcctgatgctgaaacaaaacacttcactcccttggttagatcatctgattgtggaagcaaagcactctttatacccagcgggcagatcgtcataatttccaaccttcaggagtttgttgcacttttctagaaaccaaaaaaaaaa

SEQ ID NO: 54 - DNA, 5542 bp, Homo sapiens
  misc_feature (1)..(5542): cDNA for Rho GTPase activating protein 28
agaccttgtgacaaaatagaatgttgagtttttgaggaatccaaagtaggccagtattgccagagaacaggagaggaggagagtatggtataagcatgtccagaaataaggagaacttgaagcagaatggctgcaagatgtgggtttatcaactctgatctcaggtgatgaagaggaagatggcaaagccttgctctccacattgactcgaacccaagcagctgccgtgcaaaagagataccatacctatacccaaaccatgaggaaaaaggataagcaatctatcagggatgtcagagacatttttggagtcagtgaatctcctcctcgtgatacctgtggcaaccacactaatcagctggatggcaccaaggaagaaagagagcttccaagagttatcaagacaagtggttccatgccagatgatgcttctctcaacagtactaccctgtctgacgcatcccaggataaagaagggagttttgcggttcccaggagtgactctgtggctatacttgagaccattccagttctaccagttcattccaatggatcaccggagcctggacagccagttcagaatgcgataagtgatgatgattttctggaaaagaacattccaccagaggctgaagagctgtcatttgaagtgtcttattcagaaatggttacggaggctctaaaaagaaataaacttaagaaatcagagattaagaaagaagactatgttttaactaaatttaatgttcagaaaaccagatttggcttaactgaagcaggagatctgtctgctgaagacatgaagaaaatccgccatctctctctgattgaattgactgccttttttgatgcctttggaattcaactgaaaaggaacaaaacagagaaagtaaaaggacgagacaatgggatttttggagttccacttacagtcctcctggacggtgaccgaaagaaagaccctggagtgaaagttcccctggtattacaaaaattttttgagaaagttgaggaatcaggtctggaatctgaaggaatttttcgactttcaggatgtactgctaaagtcaagcaataccgtgaagaacttgatgccaagtttaatgctgataaatttaaatgggacaaaatgtgccatagagaagctgcagtaatgttgaaagcgtttttcagagaactacccacctctctcttccctgtggaatatatacctgccttcatcagtctaatggaaagagggcctcacgtcaaagtacagtttcaagccttacacctcatggtcatggcgctgcctgatgccaacagagatgcagctcaggccctcatgacattcttcaataaagtgattgccaatgaatcaaaaaaccgaatgagtctgtggaacatttctacagtgatggcaccaaaccttttcttcagtagaagcaaacactctgattatgaagaattactgttagcaaacactgcggcccacatcatccgcctaatgcttaagtaccagaagattttgtggaaggttccatctttcttaatcactcaagtaagaagaatgaatgaagccacgatgctattgaagaagcagctcccaagtgtcaggaagctgctcaggaggaagaccctcgagcgggagactgcaagccccaagacttcaaaggtactgcaaaaatcaccctcggcaagacgaatgtctgacgtgccggaaggagtcatacgggtccatgctccacttctctccaaggtgtccatggccattcaactcaacaatcaaaccaaagccaaagacatattggcaaaatttcaatatgaaaacagtcatggttcatcagaatgtattaagattcagaaccaaaggttatatgaaattggaggaaatataggagagcattgcttggatccagatgcttatatattggatgtatatcgtataaatcctcaagcagaatgggtgattaaaccccaacaaagttcttaaaatatcctcgagagagctgctatcatgtattatatgccaaaaagatcctacattttggtagggaaaaaacaacactgtgtttgacgtatttgttcctacagcattctcagtatttctggccctcagcagtgggcattgtaagcatgaactatggaagaggcgccggttcaccaattcaactgaagctttctcatgacttttttttttataaaagtaaaggaaagatgatgtggtccttccagaagaaagaccaatctagaatatggaactctaatcacttctagtatttcaacttcctagcagaaatgaacttggccctagacctagggataagcaatgttctttatgtagccaatgctacgaaacaaaagaggtgaaagagacccttttttatacttaatgtacatatattgactttttgagcaagaatgccagaaatagccttcatttctaccctgcaaaataatccagatctgctttctaaaatgaatcagtttctaaagtgaaacatgcaatatttatgctctgactgactcctgaattggaagaggaagaacttctgtttacagaaaactgtattgttatatatgtcaggctgtgtattgtgactatcagcattctggtgcaaatgaacttttctccatcatcgactgtggaaaattgatacttttaaagcatattcttctatgagcacaggtcctcctagtgaagcttagtttgacaaagggtgtcatatgctttcctaacctgatttgtagttaacattcacagagcctacattttctcattacggttatgatgctcagtatctttccaagtgccaggcacgggcttccttttctgatcaaacataccattttttatatttcacaactatagacagtcacttctgcagtcccaatttaaaaatgcagaactgctttatccaagaatgctgaaaaatactgttctatccaggtttcctaaactataaaagcagattttgcttttgtttgttaatcataggcatggccgagcattgtggattagcctgaggcttaaaatcagatgcatgtctggtaagatgaccactgtctcactatcaagagcctgcagagccattttccagacctgtgattgcccagaacacatagtccccacgtttctaatttggagcaaatctaaaaggtgctgagggattggacagctctgactttcctcgagactatggatatagtccttctcagattagctggaaatggggacagagttctcatttatcccagatatatacattggcttttttggaatctatagattgagattttgatgaatttatattggttttaatgagacccaaatgactctctcttaacaattcaagcagtagacaaagttgctcacaaatacagaaaacatctttttgtctacttcgcaagtctcaaaacgtgataatatttaagcccatttgtagaagaaatcttgtatatactattattacaccttgatatctatacaaaacatcacttgtattaaaatatatagataactttctggcatttagaattttgttccattcatttaatttgttcatctgagaatttttttttttgcctgaaaacccattaaaagaaaacgtgccttctgtataagtttatgtttattcctacattttattcatttttaatgtatgggtaattggtggcacatgactttacataatgactttctagactctgaaaaaaatgcttttatcatttttgcaattcagacaagggtaaacattttactttatgtaattgccacatcccaagatgtaaaacggcagtaatttatgaccacgtatggaagaaattcttcaaatagctaccttacatttctgcgtatatatgaaaactgaatgatatttctcagtgtatttccttgcatttgaatggttcaattctgttgtgtttatgtagaaaattcacaaatctgttcatttaattttttaatttgagaatttttatttcttttgactgaaaacacattagaacctatgagtaaattctgaaggttttaagatttacttcagcttgtcaccagtttcaaataaatccctaataggtaacaagtaaaatacaaattcttgtctactttcatgtggttttaaatggcagggacttcgctgagtcagtaagtataatcactctagtttattcaaggatgtgtggcaactttcaacttccatacgtatatatgtatgtatggaaggccatgtcaatactagtatcattggatataactttgattcttaatcagagggcaaattcattagagaagaatttttagtagatacacagactaatatttgtgtggaggttcttttgaccaattttattcctaagaataaacaaacccctacagttaaaatgcaaagatgcctgtcatctaagtattgaaagaatttttgccttatcaagagttgttttttaaaaatttaataaattcataaaaaagactcatatctttgcaaacaaaagaagggcagatactgggcttctacatgctatccttaagagcttctccccctcacccattcctagtgctcttggtatacacttgggaaaataatgtcctccaggagaaagtgttagagggaactaacattttaggaatgcttattcagaaaaaaatcctagagttgattcattgttttcctccatcctgcaaaagaagatccctttgttaagcagctcacttggaaatatgattcttggagtcaatgatctctaaacaaactggattatcaactatttacaacgtctatacagtataaactacctcacttgcctttcttgggaaaaaaaaaatgaatggactttaacaattgttgttacaaactgtgcctgaatcttattatttaaatcacttcagttagctttcagtgtatgtttaataatatacatttaatgatgaaaaatattttcagcaaagctttaaaaccagaaatactgtgtaactgtgatctatgtggtatactctgagagaattctgtgtcctgctcattgtcttgagtttctaaccatgtgcagatgcaagcgttcaggagtaggaattaatgtccatcttttctttcaggcattttcatctgctgtccacgtatttcacttgactgaaagctcatatgagttaaaatgtcccttcttcctagcgagcatatttcaactgttcttcataaatgtttatgtgccttaaaagactttatgttaaaggattaaatagtttctctgacaggcagtttttaactgttttccacaaataaaaataatatgtcatgggattaaaatgtttgttttcaagcatttttaacagttttacacacttacatacaccttaattaaaaatttttttctgtcagacatttaccaactattttctaaaactaatttgacaaatcatgacactagaaaacgccaatgttttatgtctttgcccatctcaaaagctaatattgattcttctgttccatcagctttcattgttaagtagaatatgtatgttgcattttatccataagaaataaagagaaaaaaactaaaaaaaaaaaaa

SEQ ID NO: 55 - DNA, 1560 bp, Rattus norvegicus
  misc_feature (1)..(1560): cDNA for RT1 class Ia, locus A2
atgatggagccctgcactctgctcctgctgctggcggtcgccctgaccccgacccagacccgcgccggctctcactcgctgcggtatttcgtcaccgccgtgtcccggcccggcctcggggagccccggtacatggaagtgggctacgtggacgacacgcagttcgtgcgctacgacagcgatgcagagaatccgagatacgagccgcgggcgccgtggatggagcgggaggggccggagtattgggagagggagacccagaacgccaagagaaacgagcaggtttaccgagtggacctgaggaccgcgctccgctactacaaccagagcgagggcggctctcacaccatccagaggatgtatggctgtgacatggggtcggacgggagcctcctccgcgggtattatcaggacgcttacgacggccgcgattacatcgccctgaacgaagacctgaaaacgtggacggcggcggactttgcggcacagatcacccggagcaagtgggatcaggctggtgttgcagagagactcagggcctacctggagggcacgtgcgtggagtggctccgcagatacctggaactcgggaaggagacgctgctgcgctcagatcccccagaggcacatgtgacccttcaccccagacctgaaggtgatgtcaccctgaggtgctgggccctgggcttctaccctgctgacatcaccctgacctggcagttgaatggggaggacctgacccaggacatggagcttgtggagaccaggcctgcaggggatggaacctttcagaagtgggcatctgtggtggtgcctcttgggaaggagcagaattacacatgccgtgtggagcatgaggggctgcctgagccactttcccagagatgggagccttctccatcgaccaactccaacatggaaaccactgtcatttatgtcgtccttggagccatcattggaactctggccatcattggaattgtagtggctgttgtgaggaagaggaggagaaacacaggtgggaaaggtgactacacccctgctccaggcagggacagctcccagagctctgatgtgtctctcccagattgtaaagcatgaagacagctgcctggagtggactgagtgacagacgatgtgttcaggtctctcctgtgacacccagagccctcagttctctttagacaccagtgtctgaagttccctgtgattctatggcttctgtgtgaagaactgtggagcccagcccgccctgcacacctggaccctgtccctgcactgcctgtgttccctaccaaggccaaccttgctggtccagcctagggttcaggggtggggacatctgcatcctgtccctccctgctaccctgagcttgagctcctcacttccacactgagaataagaatctgagtgtgaacttgattgttcacatccttgacccaagtgtggatggctttttaattactggattgagaatacttagaggttttttgttttggggttttgctttgtttgattttgtttttgaataaaataaatggcagatggagaagcttcca

SEQ ID NO: 56 - DNA, 1629 bp, Homo sapiens
  misc_feature (1)..(1629): cDNA for class I histocompatibility antigen, alpha chain-like
cccattgggtgtcgggttctagagaagccaatcagcgtctccgcagtcccggttctaaagtccccagtcacccacccggactcggattctccccagacgccgagatgcgggtcatggcgccccgaaccctcatcctgctgctctcgggagccctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccccgggcgccgtgggtggagcaggaggggccggagtattgggaccgggagacacagaagtacaagcgccaggcacaggctgaccgagtgaacctgcggaaactgcgcggctactacaaccagagcgaggacgggtctcacaccctccagtggatgtatggctgcgacctggggcccgacgggcgcctcctccgcgggtatgaccagtccgcctacgacggcaaggattacatcgccctgaacgaggacctgcgctcctggaccgccgcggacacggcggctcagatcacccagcgcaagtgggaggcggcccgtgaggcggagcagtggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggagccatcttcccagcccaccatccccatcgtgggcatcgttgctggcctggctgtcctggctgtcctagctgtcctaggagctgtgatggctgttgtgatgtgtaggaggaagagctcaggtggaaaaggagggagctgctctcaggctgcgtccagcaacagtgcccagggctctgatgagtctctcatcgcttgtaaagcctgagacagctgcctgtgtgggactgagatgcaggatttcttcacacctctcctttgtgacttcaagagcctctggcatctctttctgcaaaggcatctgaatgtgtctgcgttcctgttagcataatgtgaggaggtggagagacagcccacccccgtgtccaccgtgacccctgtccccacactgacctgtgttccctccccgatcatctttcctgttccagagaagtgggctggatgtctccatctctgtctcaactttacgtgtactgagctgcaacttcttacttccctactgaaaataagaatctgaatataaatttgttttctcaaatatttgctatgagaggttgatggattaattaaataagtcaattcctggaagttgagagagcaaataaagacctgagaaccttccagaatccgca

SEQ ID NO: 57 - DNA, 3690 bp, Rattus norvegicus; identical to SEQ ID NO: 47
  misc_feature (1)..(3690): cDNA for solute carrier family 24, member 3

SEQ ID NO: 58 - DNA, 3905 bp, Homo sapiens; identical to SEQ ID NO: 48
  misc_feature (1)..(3905): cDNA for solute carrier family 24, member 3

SEQ ID NO: 59 - DNA, 1838 bp, Rattus norvegicus
  misc_feature (1)..(1838): cDNA for polypeptide N acetylgalactosaminyltransferase 1 (GALNT1)
atatgactgtgatttctgatgaaaccagattggaataattttcatsatatttgtatatttatatatatatattttaaaattttacatttgacttaaagtgccatgagaaaatttgcatattgcaaggtggttctagccacctccttggtttgggtactcttggatatgttcctgctgctttacttcagtgaatgcaacaaatgtgaagagaagaaggagcgagggcttcctgctggggatgttctagagctggtgcagaagcctcacgaaggtcctggagaaatgggaaaaccagtcgtcattcctaaagaggatcaggaaaagatgaaagagatgttcaaaatcaaccagttcaacctaatggcgagtgagatgattgcattcaacagatccttaccagacgtgaggctagaggggtgtaaaacaaaagtgtacccagatagccttcctacgacaagcgtggtgattgtctttcacaatgaggcgtggagcaccctcctgcgaacagtccacagtgtcattaaccgctcaccaagacacatgatagaagagattgttcttgtagacgatgccagcgaaagagactttttgaaaagacctctagagagttatgtgaaaaagcttaaagtgccagttcatgtaattcgaatggaacaacgttctgggttgatcagagctagattaaaaggagctgctgtgtccaaaggccaagtgatcaccttcttagatgctcactgcgagtgcacagtagggtggctggagcctctgttagcccggatcaaacatgacaggaggacagtggtgtgtcctatcatcgatgtgatcagtgatgacacttttgagtacatggcgggctctgatatgacctatggtggattcaactggaagctcaatttccgttggtatcctgttccccaacgagaaatggacagaaggaaaggtgaccggactcttcctgtcagaacacctacaatggctggaggcctgttttcaatagacagagattactttcaggaaattggaacatatgatgctggaatggatatttggggaggagaaaacctagaaatttcctttaggatttggcagtgtggagggactctggaaatagtgacgtgctcccacgttggccatgtgtttcggaaagctacgccgtacacgttcccgggtggcacggggcagatcatcaacaagaataacagacggctcgcggaggtgtggatggatgaattcaagaatttcttctacataatttctccaggtgttacaaaggtagattatggtgatatatcatcaagagtcggtctgagacataaactacagtgcaagcctttctcttggtacctagaaaatatttatcccgattctcaaattcctcgtcactatttctcattgggagagatacgaaatgtggaaacaaatcaatgtctagataacatggctagaaaagagaatgaaaaggttggaatttttaattgtcatggtatgggaggcaatcaggttttctcttacactgccaacaaagaaattagaacagatgacctttgtttggacgtttccaaacttaatggcccagttaccatgctcaaatgccaccacctaaaaggcaaccagctctgggagtatgacccagtgaaattaaccctgcagcatgtgaacagtaaccagtgcctggataaagccacagaagaggacagccaggtgcccagcatcagagactgcactggaagccggtcccagcagtggcttcttcgaaatgtcacccttccagaaatattctgagaccaaattacaacaaagaaaggattgactgggctacctcagcataagccgaattc

SEQ ID NO: 60 - DNA, 3852 bp, Homo sapiens
  misc_feature (1)..(3852): cDNA for polypeptide N-acetylgalactosaminyltransferase 1 (GALNT1)
gtgatttctgatgaaactggattggaataattttcatgatctttgtatatttatatatatatatttttaaattttgcatttgacttaaagtgccatgagaaaatttgcatactgcaaggtggtcctagccacctccttgatttgggtactcttggatatgttcctgctgctttacttcagtgaatgcaacaaatgtgatgaaaaaaaggagagaggacttcctgctggagatgttctagagccagtacaaaagcctcatgaaggtcctggagaaatggggaaaccagtcgtcattcctaaagaggatcaagaaaagatgaaagagatgtttaaaatcaatcagttcaatttaatggcaagtgagatgattgcactcaacagatctttaccagatgttaggttagaagggtgtaaaacaaaggtgtatccagataatcttcctacaacaagtgtggtgattgttttccacaatgaggcttggagcacacttctgcgaactgtccatagtgtcattaatcgctcaccaagacacatgatagaagaaattgttctagtagatgatgccagtgaaagagactttttgaaaaggcctttagagagttatgtgaaaaaactaaaagtaccagttcatgtaattcgaatggaacaacgttctggattgatcagagctagattaaaaggagctgctgtgtctaaaggccaagtgatcaccttcctggatgcccattgtgagtgtacagtgggatggctggagcctctcttggccaggatcaaacatgacaggagaacagtggtgtgtcccatcatcgatgtgatcagtgatgatacttttgagtacatggcaggctctgatatgacctatggtgggttcaactggaagctcaattttcgctggtatcctgttccccaaagagaaatggacagaaggaaaggtgatcggactcttcctgtcaggacacctaccatggcaggaggccttttttcaatagacagagattactttcaggaaattggaacatatgatgctggaatggatatttggggaggagaaaacctagaaatttcctttaggatttggcagtgtggaggaactttggaaattgttacatgctcacatgttggacatgtgtttcggaaagctacaccttacacgtttccaggaggcacagggcagattatcaataaaaataacagacgacttgcagaagtgtggatggatgaattcaagaatttcttctatataatttctccaggtgttacaaaggtagattatggagatatatcgtcaagagttggtctaagacacaaactacaatgcaaacctttttcctggtacctagagaatatatatcctgattctcaaattccacgtcactatttctcattgggagagatacgaaatgtggaaacgaatcagtgtctagataacatggctagaaaagagaatgaaaaagttggaatttttaattgccatggtatggggggtaatcaggttttctcttatactgccaacaaagaaattagaacagatgacctttgcttggatgtttccaaacttaatggcccagttacaatgctcaaatgccaccacctaaaaggcaaccaactctgggagtatgacccagtgaaattaaccctgcagcatgtgaacagtaatcagtgcctggataaagccacagaagaggatagccaggtgcccagcattagagactgcaatggaagtcggtcccagcagtggcttcttcgaaacgtcaccctgccagaaatattctgagaccaaatttacaaaaaaacgaaaaaaataaggattgactgggctacctcagcatacatttctgccacattcttaagtagcaaaaaaggaaaagtgctttcctcctctgcaggatgtaaggtttatcagccattaaaacttagacttctctagcttttcactagctgtgaaccagccttcctgtccatggacgtgaaactgcatagtaatgagactgtgcacactgatgtttacaagattgaaagagtctttctccgaaaatcatggtaaagaatactgagacaatgaaaaaaaatcaacaaaatatgctttctggagaactgtaccttttatggtttgcttgcacatcagtagtttctgctgaacgtgctgtcataatgaagagatttccaagattttttttcctgattagaactggtagccagtatattaaatattgatataaaaataaaagaactggaaccagattcagaatcatgaaaacaacatttttacaacaacaaaaaaactatattaaacagggtttaaaggaaattaaaacagaactatgagaagtacaatttgttatagtatagtatcaaatttctatatagattttatacctcagtggggaaaaataactgattccaatgacattcattttgttttcatctgtgatagtcatggatgcttttattttccttggggtgctgaaattgagctgaaaaaaaaaggctctttgaatatagttttaatttctctctacagttttttttgtttggtttgtgggctgttggaattgtaatttttaattgccttctaaaaaatggaaatttaacaatgtctgatctcagctgaacaaattagatgtttcagttgctcttgggtcaactggcttacagatttacatgtgcacacacacacaaatttcttatcacattttcgacttcttcacttgacctaactgattatgcgaaatacccaagattcatgctactgtaccacagatttgttttcacagcaataaatcttcagttctttgtttatgattccacttaacaaaaggcctgcagaagtgatttattatttgggtatttggagataatacatttgatggttttttggaaaacctttttcactccatactcagatatgcttcattgtcaaatgcatatttagattagattattgaattgtaatgtttatctgctgctttttttaaataaaatttgactgaaaatgtttaattggcattttttaatgacttagccaaagaagtgcagctattattccatattaataggcttgcatttcttttcctaaatcttatttaggctaaatcagttttatttttctctgattttttttaataccacagaatcacctgagtgtcaattgaaagttgtcaattaaaaggtaaccttttaatctcgtaggaggaatctcattaagacatttttcctgatatgtagagcagtctgttggcaaaaatgcatatattttctttcatatttgtaaaattatatttaatggaattcttttctttgattatcaaggactttcactgcaggcagtgctatttcttgtgcctaagaatgtttccaaaagtcgcatcgctaatgatatttgccaagttgagtgtacacaaagtttctcatatcctgttcaagttaatcaacatcaagcacatggggatgctttagggtgagtctatagtacaaaatgcataaaccatgtccccaggaaatttgaaaggaagcaggtgctgaatggaatttttttccttttccatgagctgtgttaattctatctccagtaggcctaatgcttgaataagcaagatgtctaatcaataaattattttcatgctcagaatttcaggtttttgtactccagcatagcttggtcttatttcttactgtatgaaagcttaacagcaatgtgatttaaggttttgttttaaatgggagatgtaagtgatttaattcatgggtacttttagaacctgatagataatcccattgcctttatttttctaattaaagaattcctaaatactttgaaaatacaaaatattcctgaataaaaaaaaaaaaaaa

SEQ ID NO: 61 - DNA, 2691 bp, Rattus norvegicus
  misc_feature (1)..(2691): cDNA for sulfiredoxin 1 homolog
cggccgcacgatggtacagcccggccgggtaaagaggccgggtggcgctgtccgccaaggaagcaatatggggctacgcgcgggtggagctctccggagggcgagtgcggggcctggggcgcccgacggtcagggacccagtggcgcgcaaggaggcagcatccactctggctgcatcgacactgtgcacaacgtgccaatcgccgtgctcatccggccgctgccgtcggtgctggacccggtcaaggtgcagagcctcgtggacacgatcctggaggaccctgacagtgtgccccccatcgacgtcctctggatcaaaggggcccaaggcggtgactactactactcctttgggggctgccaccgctatgcagcctaccagcagctacagagagagaccattcctgccaagctcgtgaggtctaccctttcagacctgaggatgtacctgggtgcatccacaccagacttgcagtagcaacctcctgataccccactcccgccttttccacaccagcaccacttcctggtgctcagaagacacctgcaggcccccgtgggcagacgttatagaaggaatatgttcttttcctacttaaggaggggtgtctgcctcttggtgcctctgcactagcccccagggactagctcccaagaatgagggggttccgttcttcagtctatgaaaagataacacggccttgaccctgatggagtaccaagaggaggaagtaagaaagatggactcggagacaaggactctccattttactctttatgatatcttatcactacatgaactaggtactaggaagtgaaggaaaacctaggtcaccgtggcttaagtgagagacgcttgttatccgcatagcctgaaccctggagcaggtgactaacacagtattatatctaacggagtgccgtatcaatgctcagtgagggcggggcctcctctcagtaatctcattgctccttgcttcgtagtcacaagatggctgctgtgtttctagaaatcatgtctgttgagaaagaaccccgatagaccaacttctgtcagaaaggaaaacctcgttcagaggtttgcacagtcgccttggtctgagctggcgaccatcgccactcacagttgcaaagaaagcagacattaggaaacgttatcacagctgagcgtggctgccttgttcattccctggaatcctggaacaggccctttgctccctgcaacacaattggggatcccattggcaggagagggaggtagcctttgctacgaaacaagccatccccagactcaacagctaaccaagtattcacgtacccgtgattctgtgggttagctaagctcctttgagcagctctactagtgtggcctggtcctgctcatgagcccagtcacttctcacttcagctggggctggttaggctggggtcacccagccattgtagcaagtgttggttgcatcggcttggttaaccttgttgcccctctagcaggtcagagcaggcttattttcatgatggcctcagggttccaagggagagtagtagtgactgagcaggacctggggcggtggctcggctgttaagagcacttacgtggtgttatgacacagtcatccctgttccagatgacctgataccctcttctagcctctgtggacacagacattacacatgtgagaaaagcacttttttcttttttctttttgagacactgggtttttctgtgtagctctggctatcctggaactcactctacagaaccaggctagccttgaactcagagatctgcctctaccacctcctaagtgcgggtgtactactgttacccagggacattttttttttcagagctgactgaacccagggccttgtgcttgctaggcaagcgctctaccactaagctaaatccccaacccctgactttttttttttaaaaggtgacaaggtttcttcagccccggtgctaggactgtgtctccactggtcagttcattccttagatttcaggggtgcaagtggacccagctctcaatgggaggaactataaagaacgtggggccatctttcatcacagatgaatcttggtggacaagagacagaatttctcactcccacacataagcactcccgttcacaagtttattgataagcacacagaggggccaagtcagcctttaagtaaggttcttaaaacctagaagaagtcgtggcacggtgccttctagagctgggtaccccactccaaggatgattcataagcagaatgaggtggataacggggcattcagaggggcttccgcaaacccagagtccacaaggcaacggaagcagcagggtgaggtggcacttgctacgcttgtcattttcaagcctctgtcttcccagctcaatagtgaggtcaccagcttccccacccactgagtgtcccagagaatgagattgaaaggaataaagtgtattgtggtgctggattgattcttaggaccaaacagagaccagggagagaaagcactttagcaggtcaaccaggactcatactgtctgggtcttagttaccttccagtttgtaatcatatctctggggtgggctggatgaagatatatgggaaccctttggtctctgcaacttttctgtgaatctaaagtcattctaaaataaaagtttatttaattaaaacacacacacaa

SEQ ID NO: 62 - DNA, 2580 bp, Homo sapiens
  misc_feature (1)..(2580): cDNA for sulfiredoxin 1
ggtatggcccggccggagttaaggccggggggaggcggcgagtcccgcggcggcggcgacgatggggctgcgtgcaggaggaacgctgggcagggccggcgcgggtcggggggcgcccgaggggcccgggccgagcggcggcgcgcagggcggcagcatccactcgggccgcatcgccgcggtgcacaacgtgccgctgagcgtgctcatccggccgctgccgtccgtgttggaccccgccaaggtgcagagcctcgtggacacgatccgggaggacccagacagcgtgccccccatcgatgtcctctggatcaaaggggcccagggaggtgactacttctactcctttgggggctgccaccgctacgcggcctaccagcaactgcagcgagagaccatccccgccaagcttgtccagtccactctctcagacctaagggtgtacctgggagcatccacaccagacttgcagtagcagcctccttggcacctgctgccaccttcaagagcccagaagacacacctggcctccagcaggctgggccatgcagaagggatagcaggggtgcattctctttgcacctggcgagagggtctgactctgggcacccctctcaccagctacaaggccttggactcactgtacagtgtgggagccccagttcccacctctgtgacaataggatcatggccttacccttgaagcattaccgagaaggagaacagagatgggcttgaagagccacgtgctgccggctccaaattcccaaggacaaggatccctctgcatttttgtctatgtaacctcttatatggactacattcagctgcaaggaaaggaaaaccttgattgcagtggtttaaacaaacagaagattgtttttccacatagcatggattctggagatgggtggctaatggtattggttcaacaactccacgaaggtaggggtcacgtcttggatccttttgccttaatctcagtgctcgttacttcatggtcccaagatggctgctgtatccccaagaatcatgtctgcgttcaaggaaggaggggtggaggaagaggaagggccaaactagctggacccgtcaccttctatcagaaagtaaaacctcgtcagaagtctgtttcctgctctctccctctgcatatcttcacttagatgcccttggcccgagccagctaccattgcacctctagctgcaaacaaagctaagacagcagggaacagaattgtcatggctgaatagaccaatcgtgttccatctactgagactggcacactgcctcctgcaataaaactgggatcccattaccaagagagaaatgcagaattgtgtaccagttagcttttgctgtgtaacaaaccatccccaaacttggcagctagaaacaaaccctgtattttcccacaatcctatgggttggcaatttgggctgggctcaacagggcagttctgctgctcacacctgggatccctcatggagctaaggtcagctgttacctcagctgggcctggatggtctaggatagccttactcacttgcctggcaggtgacaggctgttggctggaattgcttggttctcctccatgtggcctctccagcaggctagctcaggcttattcacatgatggcttcaggattccaaagagagtgagagtagaagctgaaagacttcttgagttcttggcctggaactgggactaggacagtgtcacttctgctaagttcttttggtcagagcaaatcacaaggctttacccagattcaagggatgagaaacagactacatgtcttgatgaggggaaccacaaagagcttgtggccatttttcacctatcacaaataattttggatgggtatttatttggataaaggtatttccctcttccccctttctctctgtctcatggggcctcactctgccaagttggaaggcactaagacattgtcctggccctcagggtctaggggaagaggtgttggggcaggaagtgagtctctccatgggctggacccactgtagtaggagtgcctccttgtctgcactgctggtatggggttaggccaggtaggacattccagaggggcttctgaaaaccaagagtccctggggaaagggaacagagtaaggcaggccttgttctcactgccctctaagggaacttggtcactcggcacttttaagcctcagtttctccagttcaataataaggacaagagcttttcccatgcattctctttccccgggaaagttgactgaggtgaccagtaatagaattgaaaagggagagtgtcttcagtgcaatgtggcatcctggattgggtcttggaacaaaaacaggacattagtgggaaaattggaaatctgaaaaaagtctgaattttagttaatataccaatttcagtctcttggttttgacagatgtaccatggtgatgtaagatgttgaccttggggtaggctgggtgaagggtatacaggaactctttgtactatctctgcaacttctctgtaaatctagtatcattccaaaataaaagtttatttaatttaaaaaaaaaaaaaaaaa

SEQ ID NO: 63 - DNA, 3221 bp, Rattus norvegicus
  misc_feature (1)..(3221): cDNA for ST3 beta-galactoside alpha-2,3-sialyltransferase 2
ccgtggctggccccacagtgggctgggaaaggattggccacggtgacagccgccccgtggccagcacaatgaagtgctcccttcgagtgtggttcctctctatggccttcctgctggtgttcattatgtctctgctcttcacgtactcacaccacagcatggctaccttgccctacctggactcggggactctgggcggaacacaccgagtgaagctggtaccgggctatactgggcaacagcgccttgtcaaggaggggctttcggggaaaagttgtacctgcagtcgctgcatgggtgatgccggcacctctgagtggtttgacagccactttgacagtaacatctcacctgtctggacgagagataacatgaacctcaccccggatgtccagcggtggtggatgatgctgcaaccccaattcaagtcacacaacaccaatgaagtgttggagaagctcttccagatcgtgcccggtgagaacccctaccgcttccgggacccccagcagtgccggcgctgtgctgtggtcgggaactcaggcaaccttcggggctctggctatgggcaagaagtggacagccacaactttatcatgaggatgaatcaggcaccgactgtaggctttgagaaggatgttggcagccgaacaactcaccatttcatgtacccggagagtgccaagaacctgcctgccaacgtgagctttgtgctggtgcccttcaaggccctggacctgatgtggattgccagcgccctctccacaggacagatccgattcacctatgctccagtgaagtccttccttcgagtggacaaagagaaggtccagatttacaacccagccttcttcaagtacatccatgacaggtggaccgagcaccatggccgctacccctccacaggaatgctggtgctcttctttgcactacacgtgtgtgatgaggtgaacgtgtatgggttcggggccgacagccgtggcaactggcaccactactgggagaacaaccggtacgcgggcgagttccggaagacaggcgtgcatgatgccgacttcgaagcccacatcattgacatgctggccaaggccagcaagattgaagtctaccgagggaactgagcagcctcaggcgccttcagaaccagtcacttcagggtctggcaagcgccggttgccgcccaccaatcagactgcgccgtccccagcaaccaatcagtgctgcagttgggaggagcttctttcttagccaatcacacaactcaaggagaacttccggcgccgccgtctccaccaatcatggccttgggaggcggcgggcctggaccccctcccccaacccccaaccccgctttggatgagaatttttctgctttttaagaggccggtctgggtctgggagggagcaccttgaactgctcatttccagcttagtcccgatgggtgagtgaagcccgccaaagaaatgtggtcccggcgagatactttccagggcattctgtggctcaccagctagtagggggttgtattcactccctagttgggttgacctccctagggcctcattagtgtcaggaacgggtaggaagctccaccgggtcacatgcaaggtgcttctctcacgtgtgccctcactcctgttccccaccaccttctcagcgtctgtccttaggtcctgggtctcctctccagctcacctgttgatttggagacaaaatgagaccaccgggactccgctgcttgtgctctcacacacgcggcccaagtctttagccctaaggcgagagcagcagggttggacagtaggctggaaaggagtgccctgtgcatgtatctttttctttcattgtcaccccaaagaggagcaggcagcctcttgcttgggtgttggatttgctttggattcatactttagttaccctccagctgccagggatggctatccaaccaagatcgcttgacagttgtttgcaggccttgcagatggctcggagctttgctggggaggagcagattggtatctgccatgcatggctcctcctgtccgcccctttaccatgcttactcgcctgttctaacctactttccagctcctaaagggctggtgagtcaagaatcgggttacaactggaagctatttgcatgacttgacccaggtcaaagttgaggttcatttgggagaaagggcttcagtgaatgcctcaggccccagaaaacacttcccttcagtgtccacataataatttcccattcttaatgagttgacattaacaagcagcttcctggttgggggacagtcacacgtaaggagtgtgttaaagtttggtatgaggcaaatacccctaaaaactaatatatgaagaactttgcttgaatttgttccttccaagtttcaaaagcaggggtagagaaaaagggagtataccgagaagggctgtagcggaacctctcagggctctaatcatcgtgcagtcactgtaccagtggccgttcttgcctgtctccgctctcccaagaagctgctgtgatgagaagtgggaaagtactgcgattacgacaaacagtattgagaagaagaatttatttttcctccttttgttttgcttaaaccatgagtcaccattctagaaaaggtaggtcccagcacccacatcctttttcttctacagttatttaaacattcaatttttccctctgtttcttttcctgaattaaaaagaacgaagtccatagagttactcatctgttctgaactgtacccctcataggtgattctgaggaggtaaagatgtttgggggaacaaggcaactctgataacctcttacacctgagaagccttttcatgggctctgcactccgaagtccccccacagagggctgagtattatggtttagaaaaatcaatttattatgccaggcatggtggtatgtgcctttaatcctagcagttaggagtctgcagcaggaaagtaagttaaaggctagcctcagctatatggtaactttcaaactaacctgggctatatgagaccctttctcaaggggaaaaaagggaacaaaaaaaaaaaaatccattgtcccctcttcagattcctctaaaagagtaatgccttgggaagaatttctggttcacctgaaatggccttttgtaaatacagggtctttattacagactcatgttggtttatctcagcacccataaagcggtcctcttcctgtga

SEQ ID NO: 64 - DNA, 4450 bp, Homo sapiens
  misc_feature (1)..(4450): cDNA for ST3 beta-galactoside alpha-2,3-sialyltransferase 2
cgggcggacgagagagctgcgagctaacgggtccggacgacggcggcggcggcggcggcggcggcgacggctttctgcgcgggcggcggcggcggccggagcggcgagcatggcccgcgcggcgccgggcgctgactgagggtcgtgctgcggcagcctggcgcccgacatgtgaaggggaacctgccaggggattactgtgagctcacagcaggataggctggccccgggggagtggggatgggcagaacctgggccaggagtattaggaaggagacctggaccctggtagggagctcagcacaagctcgcctgacccagatggtattggacacagtgccccgctgaacagcagctgttcccagcgccagaggaccccacagccacagtaggagctttgcgacccctacgcaactgcagctggggctggggccaccaggagaagggaccagcacctcagatgggaagcagaagggcaagacctactgcggaaaggatctcgatctctgcagcggagttttcccacactcacagagcttcctgtttttgtcctctcagggttggggtctggctcccctcctctggcagaggttttgggccaggaggtgggacaacacttgcatggaacccaagtcccctgaagcccctgtgctctctgttgtgattggagttctagggcttccccgctgccgggaacgaggggccgcctcattgtctgctcatgaaccacaaggaccccgactgctccagactggattatttcaagccgccaaagagggggacccctagagctggcagccagcaatcccaagggactagagggctgcaatggactgacctccccctcaccagggtggcaggagaggcagagcctctgtggcctagctagtgacggagagacccgatgaagccctaagcaggggccccgcctgactcagggacaggacagccactcctgccaacgtgtgttctccctacatgagggagggcgtggcaagggacccctgccactgtcccctgctgcagcacgtgcccctatgccctttgcatgtggtgccagaataggcaggctacgccgtggctggcccctcagcgggctgggaaaagagtggccacggtgaccgtcacccgcctgccggcaccatgaagtgctccctgcgggtgtggttcctctccgtggccttcctgctggtgttcatcatgtccctgctcttcacctactcgcaccacagcatggccacgctcccctacctggactcaggggccctggatgggacgcaccgggtgaagctggtgcccggctatgccggcctgcagcgcctcagcaaggagaggctctcgggcaagagctgtgcctgtcgccgctgcatgggcgatgccggtgcctccgactggtttgacagccactttgacggtaacatttcccccgtctggacccgagagaacatggatcttccaccggacgtccagaggtggtggatgatgctgcagccccagttcaagtcacacaacaccaatgaggtgctggagaagctgttccagatagtgcctggcgagaacccctaccgcttccgggacccccaccagtgccggcgctgtgccgtggtggggaactcgggcaacctgcggggctctggctatgggcaggacgtggacgggcacaacttcatcatgaggatgaatcaggcgccaaccgtgggctttgagcaggatgttggcagccgaaccacccaccatttcatgtaccctgagagtgccaagaacctgcccgccaacgtcagcttcgtgctggtgcccttcaaggtcctggaccttctgtggatcgccagcgccttgtccacggggcagatccgattcacctacgccccagtgaagtccttccttcgagtggataaagaaaaggtccagatctacaacccagccttcttcaagtatatccacgacaggtggacagagcatcacgggcggtacccttccacggggatgctggtgcttttctttgccctgcatgtgtgtgatgaggtgaacgtgtacgggttcggggccgacagccggggcaactggcaccactactgggagaacaaccggtacgcgggcgagttccggaagactggcgtgcacgacgcggacttcgaggcccacatcatcgacatgctggccaaggccagcaagatcgaagtctaccggggcaactgagccgggcctcgccgcgacccttccggcccatctatcgggcaccggggctccggcccgggacccaggaccagcaacccgcgaccaatcatgctgcagcccaggggcgtctgctgtgccccgccaatcacgagactgggggaccggccgggcctggcaccaatctgcgctgcggtcgggcggagcttctgtttctcccagccaatcatgtgactcaaggaaaacttccggcgctgtgtccagtctcctccaatcaatggccttcgggggcgggccagcggccgctcaatccccactccctcatgctttgggttagggttttctttcacgctttctgaggaggagagcatggcgcgggcctcggcgaagcacttccgtcagcctcgggcggaggatcgtcttagtagctggccagacctggaggagagcgggcggtctttgcgggcctcaggagataggtggccgcccggcccctttccccgcctgcccggataggtgcgtgcagacccaccaaagaaaagcggtcgctgcgggacccccagtcccacctcggccgcgctgtgggatggctgcgcccccagggcttcctgcgtctccaggaagccgggagtggcgccctctgcggggtggggtgctcccgggccgaactccggacaggtggaggggacagggcagagctcgaggaagccctgtccccttcagtgcaaggtgctgtcactcacgtgtgccctcgaccctcccgttcacccgcagccttctcagcgcctctccctgggccggaggcctcctcaccagcctacctgttgctctggaaaaaaatcccgtcccccgactccgtccctacccccagtcttcggccggctctggcccctggggagggggctgcacggcggaaggaggctggctatgggcccggctgcccgctgcatgtacctcctcctccacccatcgcctcttgcctgggggtaactttgcctggggctcattctttggttaagctgaagctgccgtgggtggccaaaccgcagattctttgcaaattctgagctggcagagctcgcagccgggagccggccggggaagaggagacttgcgcgccgcaagccgcctgcctccaccctgctctccatctcccgctctagaagggctgggaagctcgcggccggggttccacctggaagctgcttgcatggctgaacccagcttaggtccctgacggggctgctggtggaattctcccccttcgaagctggggaggtttaggagggggaaggcttctgtgaagctctcaaaccactaatagagccccctccccaacagtgacggcgcagatgctcccccttttcttagttgacaccaccaggcagcttcctggccgttggtaggttcctgcagctggctgagggaacagggaccggcaggggactttgttaggggagggttgggatgggcagtgggcccctgaaagttaatatattggaacctagctcgagtgtcgttctttccaattccgaaagtagaaagagtaaaaataggggtgattggggtggggttagtagaatgcctctctcagggcgctcccccctcccccaccgttttagagagctaggcctcagccagtcttgccactcccatctcagtgcttcctgaagaggctgttttgagtgttgatgaaaagcaatgcaattatgccaaacagtattgagcagaataatttatttcttttttttcttttgctttaaatcatgaatcccgccaggtacggtggctcacacctgtcatcccagcactttgggaggccaaggcgggcggattacttaatacttaaggtcaggagttcgagaccagcctggccaatatggtgaaacctcgtctctaccaaaaaaaaaaaaaaatacaaaaattagccaggcgcagtggtgcgcacctgtaatcccagctacttggaaggctgaggcaggagaatcccttgaaccgaggaggcgaaggttgcagtgagccgagattgtgccactgcactccagcctgggcgacagagcaagaccctgtctcaaaaaaattaaatcatgaatccccatcctggaagaggtaggtcccagcatccagccagattttctgcaatagtaatttaaacacacttttttatttccttccctttctgaattaaaaggaacaaaaaactgtctagtccttcattgttctgaaatgggcatgatggaagaaggcgggtctggtaggatagctgtgactgataatg

SEQ ID NO: 65 - DNA, 3367 bp, Rattus norvegicus
  misc_feature (1)..(3367): cDNA for C2 calcium-dependent domain containing 2-like
cggagccggtgccggcccgcgagccccggtgtctccgcagactggtctctgccggtgtccggcgggggcccaagccgtgggaggagtcgggtgctagctggagtctagctggggtcacaacgccggtgtcagggacccggatctccggctacggagacagagcccgagcgtcgcgaccccggagaacctcttctctatcggtggcaccgactggtcctaggatctcggcggcgccggagagtcccggagcccgcgctctcagccccgggggagctcgcccacgccccacggtccatccgggccatgctcccccggggcagcggctgagcccgagccgggaccagagcccgcgggagcatggatccagactgggggcagcgggatgtgggctgggcggccctgctggtcctcttcgccgcctcgctgctcacggtattgggctggttgctgcagtatgcccggggtttgtggctgtcgcgagccggtgggggccgagactctcgacccgcttcagctgccgagcccgggggttcattgcgcgagctgggtgtgtggcgttcgctgctgcgtttgcgggcgacccggagcagcgcccccgaggaagccggcgtacggggcctcctggcttcgctctttgccttcaagtctttccgggagaactggcaacgggcttgggtgcgagccttgaatgagcaggcctgcagggacgggagctccatccaaatcgcctttgaggagataccccaactcccaccaagagccagcattagtcatgtgacctgcgttgaccaatcagagcgtaccatggtgctgcactgccagctggctgctgaggaggtgcgcttccccatctccgtgacccagcagtcccccgctgccgtctccatggagacctaccacgtcactctgacactgccaccaacacagttggaagtcagccttgaggaaatccctgatgaggggctgctggtgtcctgggccttcacggaccgcccagatctcagcctgaaggtgcttcccaagtctcagactagggagagagatgaggaacaaccagagctttcaacagttgaggaactgatcaaggacgctatcgtcagcactcagcccgccatgatggtcaacctcagggcctgctctgccccaggaggcctggtacccagtgagaagccacccacgatgtcccaggcccagccatccatccccagacctacccgactattcttacggcagcttcgagcatctcacctgggaagtgagctaggaggtactgaggaactgtgctgtgctgctgagcttgacaaccccatgcaacagaagtggaccaaacccacaagggctggctctgaggtggaatgggcagaggatttagctctggatctgggtccccagagccgggagctgaccctcaaagtgctcaggagcagcagttgtggagatgctgaactccttggccaagccacactgcctgtgggctcaccttccaaaccactgtcacggaggcaagtgtgtccactgactccagggcccgggaaatccctgagcccagcggccatcgtgacagtggagctacactatgagcagggctctcctcggaatctgggcacgcccacctcttccacccctcgccccagcatcacacccaccaagaagattgagttggaccggaccatcatgcctgatggcacaatcgtcaccactgttactactgtgcagtcccgcccccgtgtagatggcaaactagactccccctcccgctccccgtccaaggtggaggtgactgagaagatgacaactgtgctgagtgagagcagcggccccagcaatgcctcccacagtagcagccgggagagccacctttccaatggcttggatccagtagcagagacagcaatccgccagctgacggagcccagtgggcgggcagccaagaagacacccaccaagcgcagcacgctcatcatctctggtgtttccaaggtgcccatcgctcaggacgagttggcactctccttgggttatgcagcatctctggaagcctcaatgcaagatgatgcaggagccagtggtggtccttcatcacctccctcagacccctcagccacatccccaggacccgttgatgctctctccagtcccacaagtgtccaggaagcagacgagacaacacgttcagacatttctgagaggccgtcggtggatgatgttgagtcagaaacagggtctactggtgccctggagacccgaagcctcaaggatcacaaagtgagtttcctgcgaagtggcactaagctcatcttccgccggcggccccgacagaaggaagcgggtctgagccagtcgcacgatgacctgtccaacacaacagccacacctagtgtccggaagaaggctggcagcttttcccgtcgccttatcaagcgtttttccttcaagtccaaacccaaggccaatggcaaccctagcccccagctctgagagctcctgaactaggagagggcaagagttctcttagtttgtcccccacatccccttctgtaccccttcttggatccccagtgctgggaccaggaaagcccttgggttataggaaaccccagcactctgggctgtggggttagcgggaagggtgtctcacatgggaagcatttgagaggcaacagttgttgctgtggacataggagagtttggcagctgagagtaaagggcaggctctgtctctcttgtgtggacattcttcagaagcctctgcagtctgctggtactgcccccagaggggagtccagagcactctcttcagcagatccttctcccttatttattctcttttctatttatatatgtggtctagagcccttggcaaacagatgatagagggcatctctcccaggtgacccttttctgtcccaggagggtaaggcaattcccaggacaggtttctacacctcccttgggagtcctcttttagaccagcaccgatgtctgtctgccttcagaagacattggcagatcataggaagccggcctagtgcccgggatggggggcaggcactccccacctccctacctcccacacgcaccttgtctctccttcccctttattaccattttgtacttgataccttctccatgagcagtggcgcagttggaaggaaggagccaggaagctgggagcaagccttctttccctagagagatggctttaggagctttagacagtatggtcataaagcaaggttgcacctctatatgttgggagatgatgatgatgatgtccattgctgtgtgatggcttggaatttaatttatttaattaaaatcaaattggaatttacaaaaaaaaaaaaaaaaaaaaaaaaaaaaaaaa

SEQ ID NO: 66 - DNA, 3370 bp, Homo sapiens
  misc_feature (1)..(3370): cDNA for C2CD2-like
gagccggtgccggcccgcgagccccagcgtctctgcagaccagtcccctccagggtccggcggggcccgcgcggtgggaggagtcgggcactggccggcgactaacgggtccgactgctgaccaagctaggagagacccggaccacggagacagagaccccggcatcgcgacccccgaggacctcctctcctcgccctgtggcacccactagtcctgggcactcagccgcggagagcccccgaccccgcgcgcccagccccgggggagcccacctcctccccgcggcccgcccgggccatgctcccccggggcagcgggtgagccccagccgggaccgggatcggagcccgcgcggagcatggatccgggctgggggcagcgggacgtgggctgggcggccttgctgatcctcttcgccgcctcgctgctcacggtgttcgcctggctgctgcaatatgcccggggcttgtggctggcgcgggcccgcggggaccggggcccgggacccgccttagccggggaacccgcgggttccctgcgggagctgggcgtgtggcgctcgctgctgcggctgcgggcgactcgggctggcgccgccgaggagccaggagtccggggcctcctggcgtcactcttcgccttcaagtctttccgggagaactggcagcgggcttgggtgcgagcgctgaacgagcaggcctgcagaaacgggagctccatccaaatcgcctttgaggaggtgccccaactcccacccagagccagcatcagtcatgtgacctgcgtagaccaatctgagcataccatggtgctgcgttgccagctctctgctgaggaggtgcggttcccagtctctgtgacccagcagtcccccgctgccgtctccatggagacctaccacgtcactctgacactgccaccaacacagttggaagtcaacctggaggaaatccctggtgaggggctgctcatatcctgggccttcactgatcgcccagatctcagcctaacggtgcttcccaagcttcaggccagggagagaggtgaagaacaagtggagctctccacaattgaggaactgatcaaggatgccatagtcagcacccagccagccatgatggtcaacctcagggcttgctctgccccaggaggcctggtacccagtgagaagccacccatgatgccccaggctcagccagccatccccagacctaaccggttattcctacggcagcttcgggcatctcacttgggaaatgagctggaaggcaccgaggaactgtgctgtgtagctgaactcgacaaccccatgcagcagaagtggaccaagcccgcgagggctggatccgaggtggagtggacagaagacctggcactggatctgggcccccagagccgggagctgaccctcaaagtgctgaggagcagcagctgtggagacaccgaactcctaggccaggccacactgcctgtgggctccccctccagaccactgtctcgaagacagttgtgcccactcaccccagggccagggaaagccctgggaccagcagccaccatggcagtggagcttcactatgaggagggctctccccggaacctgggtactcccacctcctccactccacgccccagcatcacacctaccaagaagattgagcttgaccggaccatcatgcccgatggcaccattgtcaccacagtcaccactgtccagtcccggccccgtatagacggcaaattagactccccctcccgctccccgtccaaggtggaggtgaccgagaagacgacaactgtgctgagtgagagcagtggccccagcaatacctcccatagcagcagcccaggggacagccacctttccaacggcttggaccctgtagcagagacagcgattcgccagctgacagagcccagtgggcgggtggccaagaagacacccaccaagcgcagcactctcatcatctctggtgtttccaaggtgcccattgctcaggacgagttggcgctatccctgggctatgcggcatccctggaagcctcagtgcaggatgatgcagggaccagcggaggcccctcttcacctccctcagacccaccagccatgtctccaggaccgctagatgccctctctagtcccacaagtgtccaggaagcagacgagacaacccgttcggatatttctgagaggccatctgtggatgatattgagtcggaaacggggtccactggtgccctggagacccgcagcctcaaggatcacaaagtgagtttcctgcgcagcggcactaagctcatcttccgccggaggcctaggcagaaggaagctggcctgagccaatcacacgatgacctctccaacgcaacggccacgcccagtgtccgaaagaaggccggcagcttttctcgccgccttatcaagcgcttttccttcaaatccaaacccaaggccaatggtaaccccagcccccagctctgaggacccagctctgaaagggcacgagttctctcagcccattccccacctccccttccataccccttcctggatctccagtgcctgggccaggaaagccctctgggttccgggaagccccgtccaccctgggccatggggccggttggaaggatacttggaacgggaagcacatgagaggtgggcacccggtgccgaggacatggacgagggactggtggctgggagggagaggagggccctgtccggcatgtgtgggtattccccagaagcatttgcctcctgctgagcctggtccctgagcggagtcccagggtgctcagctcttcagctgacccttcttcccttatttattctcttttctatttatatgtgtggcttaggaccctccgtgaacagatgatagagggcatctctcccaggtgacccttcttttctgtcccaggagggtgggtaattccctttgggatggggctcccacacctccctcaggtccccactcagaccagcaccagtgtctgcctctgagaatgttggcagctcacagagagcagggccggcccgggatggggggcaggtactccccaccttcctgcctcccctcctgctcctcatccctccctccccctttattaccgttttttgtacttgatgccttctctgtgagcagtggctctgtgggaaggagggagccgggagcctggtgggaagccttccccagagagatggctttaggggctttatttaaagactgtgatgatggagccacgcaaggctgcacctctgtgtgttgggagacgatgatgatgtccattgctgtgtgatggcttggaatttaatttattaaagtcaaattggagtttaaaaaaaaaaaaaaaaaaaaaaaaaaa

SEQ ID NO: 67 - DNA, 5918 bp, Rattus norvegicus
  misc_feature (1)..(5918): cDNA for heat shock 70kD protein 1B
gaattccagcacttgggtgtcaaggctggaggagcaagagtttaaggctagcctgggctacatgagcccctattttgaaaaagaaaacaagaaataaaaaagttagaaaaaagaaaaaatgaaaacaaaagatgtgtgcgtgtgtatgtgtgtgagagagagagacagtctctctctctctctctctctctctctctctctctctctctctctctctctctgtgtatgtgtgtgtgtgagacaattactccctgctgtggactgttacctcagaactaagactctaatgcaatctattctttttttaaaaaacaggctcggggctggggatttagctcagtggtagagcgcttacctaggaagcgcaaggccctgggttcggtccccagctccgaaaaaaagaaccaaaaaaaaaaaaaaaccaaaaacaaaaaaaaaaaacaaaaaaaaacaaaacaaaacaggctctaatgtagattgggctagcctcaaactcttcatcctcctgcctccccctccaaagcatgaggactacagaggtgggccaccatacccagtaacctcatactgtttcaagacatctaactagggggtctttaattcgtacacggaaggaggagcccggaagaagaaaaagggacatcactgacagctaaacatatttataatgagaggaggtaggggacagacaagggacggaaaggtgatgtctaggacaaaaagctaaccacataaactagtaggtgggtcccgacacctctgcttagggtctcacccaaaagtcggagttaacagcactgtgaggccagatgctatcagaggacaggacacaccatctctccacctaggaaaggtgaactgacaggggcagcagacacagaagaatcagcaaactcttaagtcaaccctgaggttactacagcacagaatagaacagtgcctggcaaggagcagctgtgtgaaggagaagccaagcagccatcctcagcattcttcttctattaatcttttgggacagggtctgcctctgtaacctaggctgaccttgactcgctgcagtccctctgccccaccctctaccgaaagtttttttttttctcctttctaatgccaggctgccgcctgaggcaaagggagcggcttgcagcctggcacggtcggtcgattaaaccttgctctccccgtcctgggacactttccttttttgggtcacaggtcctcctaacatgagaaccgagtgtttacacaatgatgttcttttgaaaaccgtgaaaactccacaggcgatgtacttgtacgttaagcgtgacataaagacagcaaagcgaataaactatactgcaagatctcttctctttccctatttaaacctaaaatggagggagtggggggcagacacagacaggcgagcattccacaggcgccccccccacgctgtcacttccaggcaggacccaatcacagacttcttagccaagcgttatccctcccgttttgagaaactttctgcgtccgccatcctgtaggaagaatttgtacaccttaaactccctccctggtctgattcccaaatgtctctcaccgcccagcactttcaggagctgacccttctcagcttcacatacagagaccgctaccttgcgtcgccatggcaacactgtcacaaccggaacaagcacttcctaccaccccccgcctcaggaatccaatctgtccagcgaagcccagatccgtctggagagttctggacaagggcggtaccctcaacatggattactcatgggaggcggagaagctctaacagacccgaaactgctggaagattcctggccccaaggcctcctcccgctcgctgattggcccatgggagggtgggcggggccggaggaggctccttaaaggcgcagggcggcgcgcaggacaccagattcctcctcctaatctgacagaaccagtttctggttccactcgcagagaagcagagaagcggagcaagcggcgcgttccagaacctcgggcaagaccagcctctcccagagcatccccaccgcgaagcgcaaccttctccagagcatcccccagcggagcgcacccttccccagagcatccccgccgccaagcgcaaccttccagaagcagagagcggcgacatggccaagaaaacagcgatcggcatcgacctgggcaccacctactcgtgcgtgggcgtgttccagcacggcaaggtggagatcatcgccaacgaccagggcaaccgcacgacccccagctacgtggccttcaccgacaccgagcggctcatcggggacgccgccaagaaccaggtggcgctgaacccgcagaacaccgtgttcgacgcgaagcggctgatcggccgcaagttcggcgacccggtggtgcagtcggacatgaagcactggcccttccaggtggtgaacgacggcgacaagcccaaggtgcaggtgaactacaagggcgagaaccggtcgttctacccggaggagatctcgtccatggtgctgaccaagatgaaggagatcgccgaggcgtacctgggccacccggtgaccaacgcggtgatcaccgtgcccgcctacttcaacgactcgcagcggcaggccaccaaggacgcgggcgtgatcgcgggtctgaacgtgctgcggatcatcaacgagcccacggcggccgccatcgcctacgggctggaccggaccggcaagggcgagcgcaacgtgctcatcttcgacctggggggcggcacgttcgacgtgtccatcctgacgatcgacgacggcatcttcgaggtgaaggccacggcgggcgacacgcacctgggcggggaggacttcgacaaccggctggtgagccacttcgtggaggagttcaagaggaagcacaagaaggacatcagccagaacaagcgcgcggtgcggcgactgcgcacggcgtgcgagagggccaagaggacgctgtcgtccagcacccaggccagcctggagatcgactctctgttcgagggcatcgacttctacacgtccatcacgcgggcgcggttcgaggagctgtgctcggacctgttccgcggcacgctggagcccgtggagaaggccctgcgcgacgccaagctggacaaggcgcagatccacgacctggtgctggtgggcggctcgacgcgcatccccaaggtgcagaagctgctgcaggacttcttcaacgggcgcgacctgaacaagagcatcaatccggacgaggcggtggcctacggggcggcggtgcaggcggccatcctgatgggggacaagtcggagaacgtgcaggacctgctgctgctggacgtggcgccgctgtcgctgggtctggagaccgcgggcggcgtgatgacggcgctcatcaagcgcaactccaccatccccaccaagcagacgcagaccttcaccacctactcggacaaccagcccggggtgctgatccaggtgtacgagggcgagagggccatgacgcgcgacaacaacctgctggggcgcttcgagttgagcggcatcccgccggctcccaggggcgtgccccagatcgaggtgaccttcgacatcgacgccaacggcatcctgaacgtcacggccactgacaagagcaccggcaaggccaacaagatcaccatcaccaacgacaagggccgcctgagcaaggaggagatcgagcgcatggtgcaggaggccgagcgctacaaggcggaggacgaggtgcagcgcgagagggtggctgccaagaatgcgctcgagtcctatgccttcaacatgaagagcgccgtggaggacgagggtctcaagggcaagatcagcgaggctgacaagaagaaggtgctggacaagtgccaggaggtcatctcctggctggactctaacacgctggctgagaaagaggagttcgtgcacaagcgggaggagctggagcgggtgtgcaacccgatcatcagcgggctgtatcagggtgcgggtgctcccggggctgggggcttcggggcccaggcgcccaagggaggctctgggtcggggcccaccatcgaggaggtggattagaggcttttctggctctcagggtgttggctagagacagactcttgatggctgctggtgcacgattcttatcaagttactccttctctccggagttcagtttaaagttacagccttttatacggtaattgatttgagtttgttacattttgtatgctcgtgggttttttatatattcaaattaaggttgcatgttctttgcgtttaatctaagtagctgtgtaaaaatggtgtttccttcctgcgaacacctcagcactgccaccctgtgtacagttttttccttgcatccctacaaactgagaaaaaaagttatcttttgtaacttaaacattcaaaataaaatgttacaagtatgttttgtccgtgtgtatgttgggagggctaatggattctgggttcatgtggatttcttagctttgcgatgacggggaaatggggtttgggtactttggttagaagtgtgggttttggggttggggattgagctcagtggtagagcgcttgcctaggaagcgcaaggccctgggttcggtccccagctccgaaaaaagaaccaaaaaaaaaaaaagtgtgggttttcagcgcagttatgcagaaaatgcaggtattcggtttgagggtaggactcattggggggaacagtagggtggtgctgtagttgggggtcagtggaagagggactcttagggctatgatgactcagttctcttagccctccagggctgaaccagaactgtttcccaacttatgggtcgagacctctgttggagtcagacgacctttccacagtggtagcctcggacattgaaaattgcaggggttgttggttgtcggttgggtttttgttttgtcttgttttgtttttcaagacagggtctctctgtgtagccctggctggcctcgatttcggatccacatgcctttcccaaacctggcttccttattacttttttttttttttttaagatttatttacttattatatataagtacaatgtagctgtcttcagagacaccagaagagggcatcagatcccattacagatggctgtgagccaccatgtggttgctgggatttgaactcaggacctcttcaagagcagtcagtgctcttaacttctgagccctctctccagccctttattacttttagagatttcttaccaggttgccctggctgtacttacactttctctttagctctgctgagctttcaattcaccagttccagaccacacccctgaacccaccagctgaagacaacatgtcttgctatctttttctttcctttttggggcagggatggtctgtgtgttgtgtgtgtgagagacagaggcagagacatccttcccatgtagccctaactgagtcggaagtcattatttagaccaggctgccctgactcctgtatctgcctctccaatggattatgggcacctgccacctcacccactcactttatccccaggatccccctccctcttcccaggcagtaggtctggtgatggagaagaccacttaccatgtccatggctcaaagaagcaggcagaacgtaggggaaagagttgctttcacctcaagcctttggatccatgggtgttcattctagtggggtaatgccatcctttgagagctggggggtgtgggtcaacagagatcggttgctatgtgtgcatgaggcccgcaattcagtccctggctccatggagaacgaaccaatcagttgggtgaggtgtaaaccacatgtaattttagcatgtccaaacagacacgttaaggacatcctagggtaaagaagtcttcagcaatagtttaattgaggcaattatgctcatgaattc

SEQ ID NO: 68 - DNA, 2551 bp, Homo sapiens
  misc_feature (1)..(2551): cDNA for heat shock 70kDa protein 1B
ggaaaacggccagcctgaggagctgctgcgagggtccgcttcgtctttcgagagtgactcccgcggtcccaaggctttccagagcgaacctgtgcggctgcaggcaccggcgtgttgagtttccggcgttccgaaggactgagctcttgtcgcggatcccgtccgccgtttccagcccccagtctcagagcggagcccacagagcagggcaccggcatggccaaagccgcggcgatcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcggctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtgctcatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtggcctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggcccccaggggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggcggacaagaagaaggttctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctggcggcttcggggctcagggtcccaagggagggtctgggtcaggccctaccattgaggaggtggattaggggcctttgttctttagtatgtttgtctttgaggtggactgttgggactcaaggactttgctgctgttttcctatgtcatttctgcttcagctctttgctgcttcacttctttgtaaagttgtaacctgatggtaattagctggcttcattatttttgtagtacaaccgatatgttcattagaattctttgcatttaatgttgatactgtaagggtgtttcgttccctttaaatgaatcaacactgccaccttctgtacgagtttgtttgttttttttttttttttttttttttgcttggcgaaaacactacaaaggctgggaatgtatgtttttataatttgtttatttaaatatgaaaaataaaatgttaaactttaaaaaaaaaaaaaaaaaaaaaaaaaaaaaaa

SEQ ID NO: 69 - DNA, 1162 bp, Rattus norvegicus
  misc_feature (1)..(1162): cDNA for alpha-1-microglobulin/bikunin precursor
gaattccggactgcagagccatgcagggtctcggggccctgttcttgttgctgactgcctgcctcactttgaaggctgacaatgtgccaacactgccagatatccaggttcaggagaacttcaatgaggcccggatctatggaaaatggttcaacctggcggtgggctccacctgcccgtggctgaggcggattaagaacaagatgagcgtgagcacactggtgctgcaggagggggcgacagaagccgagatcagcgtgaccagtactcaatggcggaaaggtgtctgcgaggagatctccggggtgtatcagaagacagacattgacggaaagttcctttaccacaaatccaaatggaatgcaaccttggaatcttatgtggtccacaccaactatgacgaatatgccattttccttaccaagaagttcagccaccgccacggacccaccatcactgccaagctctatggccgggaaccacagctgagggacagccttctgcaggagttcagggaggtggccctgagtgtgggcatccctgagaactccattgtttttatggccgacagaggagaatgtgtccctggggatcgggaggtagagtccacttcatttgcaagagcccggcgggcagtgctgccccaagagaatgaagggtcagggagtgagccactcataactgggaccctcaagaaagaagactcctgccagctgaactactcagaaggtccctgcctggggatgcagcagaagtattactacaatggcgcctccatggcctgcgagaccttccagtatggcggctgcctaggcaatggtaacaacttcgcctccgagaaggaatgcctgcagacgtgccggaccatagcggcctgcaatctccccatagtccaaggcccctgccgagcctttgcagaactctgggcatttgatgcagcgcaagggaagtgcatccaattcatctatgggggctgcaaaggcaacggcaacaagttctactctgagaaggagtgcaaggagtactgtggagtccctggtgatgggtacgaggagctaacacgcagttgaaggagccggtctgcaagccagagggtagccacggtccgtcactgagcagtccagcttagatgatctggacccaataaaacaaggcggaattc

SEQ ID NO: 70 - DNA, 1450 bp, Homo sapiens
  misc_feature (1)..(1450): cDNA for alpha-1-microglobulin/bikunin precursor
attgtgcttggccaatgcctcttctgaagcagccatcccggcctcttggtactgctgaccccagccaggctacagggatcgattggagctgtccttggggctgtaattggccccagctgagcagggcaaacactgaggtcaactacaagccacaggccccttccccagcctcagttcacagctgccctgttgcagggaggcggtggcccttctgttgctagaccgagcctgtgggatataccaaggcagaggagcccatagccatgaggagcctcggggccctgctcttgctgctgagcgcctgcctggcggtgagcgctggccctgtgccaacgccgcccgacaacatccaagtgcaggaaaacttcaatatctctcggatctatgggaagtggtacaacctggccatcggttccacctgcccctggctgaagaagatcatggacaggatgacagtgagcacgctggtgctgggagagggcgctacagaggcggagatcagcatgaccagcactcgttggcggaaaggtgtctgtgaggagacgtctggagcttatgagaaaacagatactgatgggaagtttctctatcacaaatccaaatggaacataaccatggagtcctatgtggtccacaccaactatgatgagtatgccattttcctgaccaagaaattcagccgccatcatggacccaccattactgccaagctctacgggcgggcgccgcagctgagggaaactctcctgcaggacttcagagtggttgcccagggtgtgggcatccctgaggactccatcttcaccatggctgaccgaggtgaatgtgtccctggggagcaggaaccagagcccatcttaatcccgagagtccggagggctgtgctaccccaagaagaggaaggatcagggggtgggcaactggtaactgaagtcaccaagaaagaagattcctgccagctgggctactcggccggtccctgcatgggaatgaccagcaggtatttctataatggtacatccatggcctgtgagactttccagtacggcggctgcatgggcaacggtaacaacttcgtcacagaaaaggagtgtctgcagacctgccgaactgtggcggcctgcaatctccccatagtccggggcccctgccgagccttcatccagctctgggcatttgatgctgtcaaggggaagtgcgtcctcttcccctacgggggctgccagggcaacgggaacaagttctactcagagaaggagtgcagagagtactgcggtgtccctggtgatggtgatgaggagctgctgcgcttctccaactgacaactggccggtctgcaagtcagaggatggccagtgtctgtcccggggtcctgtggcaggcagcgccaagcaacctgggtccaaataaaaactaaattgtaaactcctgaaaaaaaaaaaaaaaaaaa

SEQ ID NO: 71 - DNA, 4620 bp, Rattus norvegicus
  misc_feature (1)..(4620): cDNA for murinoglobulin 1
taagctctgtcctcctgtcctcctgtcatcctgtcctcctgtccttccatcatgaagaagaatagagaggctcagctgtgccttttttcagctcttcttgccttcctgccctttgcttccttgctcaatggaaactcgaagtatatggtgctggtcccttcccagctctacaccgagacccctgagaaaatctgcctgcatctataccatctgaatgagacagtgactgtgacagcttccttgatatctcaaaggggaacgagaaaactgttcgatgagcttgtggttgacaaggacttgttccactgtctttccttcactatccctagattgccctcttctgaagaggaggagtcactcgacatcaacattgaaggggcaaagcataaattcagcgaaaggcgtgtggtgcttgtgaagaacaaagaaagtgttgtctttgtccagactgataagcccgtgtacaagccaggacagtcagttaaatttcgggttgtctctatggataaaaatctacaccccctgaatgagttgtttcctctggcttacattgaggatccgaaaatgaaccgaattatgcagtggcaggacattaagacagagaatgggcttaagcaattgtccttcagcctgtcagcagagcccattcagggcccctacaagatagtgattctcaaacagtcaggagtgaaggaagagcattccttcaccgtcatggaattcgtgcttcccagatttggtgtcgatgtgaaggtcccaaacgccatctctgtgtacgatgaaataatcaatgtgactgcctgtgcgatatatacctatgggaagcctgttccaggacatgtaaagataagtctttgccatggaaatccttcctttagctctgagacaaagtctgcatgcaaagaagaggactccgagctagacaacaacggctgcagcacacaagaagtgaacatcactgagttccaattaaaggaaaattacctcaaaatgcaccaggctttccatgtgaatgcaactgttacagaagaagggacagggtcagagttcagtggatctggaagaattgaagtcgaaagaaccagaaacaaattcctatttctgaaagcagactcacacttcagacacgggatcccattctttgtgaaaatccgcctagtggatatcaagggagatcctatcccaaatgagcaggtcttcatcaaagcacaagaagctggctacaccaatgctactaccactgatcagcatggcttggcaaagttctccatagataccagcagcatctcaggctattccctcaatatcaaagtctaccacaaggaggaaagttcatgtatccattcctcttgcacagcagaaagacacgcagaggaacatcatacggcctatgctgtttactccctcagcaagagctacatctaccttgacacagaagctggcgtcttgccctgcaaccagatccacacagttcaggcgcattttattctgaaggggcaggtcttgggagtgctgccacagattgttttccactacctggtcatggcccagggcagcatcctccagactggaaaccacactcatcaagtggagccaggagtatctcaagtacaaggcaacttcgccttggagatccctgtggagttcagcatggttcccgtggctaaaatgctcatctacaccatcttgcctgatggagaagtgattgcagattctgtaacgtttcaagttgaaaaatgtcttcgcaacaaagtgcacctgagcttcagcccatcccaaagtcttccagcctcacaaacccatatgcgggttacagcttctcctcagtccctctgtggcctgagagctgtggaccaaagcgtgctgctgctgaagcctgaggctgagctctccccttccttgatatatgatctaccaggtatgcaagacagcaacttcattccaagttcttatcatccatttgaagacgaatatgattgtttgatgtaccaaccaagagacactgaagagctcacatactcagtaccatatggacgagagaaggatgtctacagatatgtgagggacatgggtttaacagcattcaccaacttgaagatcaaacatcctacatattgttatgaaatgaacatggttgtactatcagcacctgctgtggaatctgaactttcaccaagagggggagagtttgaaatgatgcccttgggagtaaataaaagtccactacctaaagagccaccacgtaaagatccaccacctaaagatccagttattgagaccattaggaattattttcctgaaacctggatctgggatttagtcacagtaaactcctcaggagtgactgaagtggaaatgacagtccctgacaccatcactgagtggaaggctggagccctctgcctgtctaatgacactggtcttggcctctcttctgtggcaactctccaagccttccagcccttctttgtggagctcacaatgccctactctgtgatccgtggagaagccttcatgctcaaggccacagtgatgaactacctccctacaagcttgccgatggctgtgcagctggaagcttctcctgatttcacagctgtcccagtgggaaatgaccaagattcttattgccttggtgccaatgggaggcacacctcatcctggttggtaactcccaaatctttagggaatgtgaatttctctgtgtctgtggaagcacaacagtccccagagctctgtggttctcaggtagccacagtgcctgaaaccgggagaaaggacacagtagtcaaagtcttgatagttgagcctgaaggaatcaagaaagagcataccttcagctcacttctctgcgcatcagatgctgagctgtcagaaacattgtctctgctgcttccaccaacagtagtgaaagattcagccagagcacatttctctgtgatgggtgatatcttgagttcagccataaaaaacacacaaaatcttatccaaatgccctatggctgtggggagcagaacatggtcctttttgctccaaacatctatgttctgaaatatctgaatgaaactcaacagctgacagagaagatcaagtccaaggcccttggttacctcagagctggttatcagagagaactgaactacaaacacaaagatggctcctacagtgcttttggggatcacaatggccagggtcaaggaaacacttggctcacagcctttgtgctcaagtcttttgcccaagctcgagccttcatcttcatcgacgaatcacacatcaccgacgccttcacctggctctccaagcagcagaaggacagtggctgcttcaggagctccggatcactgttcaacaatgccatgaaggggggagtcgatgatgaaatcaccctctctgcctatataaccatggcccttctggaaagttcactcccagacacggatcctgttgtctccaaagccctgggctgcctagaggcatcttgggagaccatagagcaaggaagaaatggcagctttgtgtacaccaagacactgatggcctatgctttcgctcttgcgggaaaccaggaaaagagaaacgaaatcctgaaatcccttgataaggaagctataagggaagacaactccatccactgggaaagacctcagaaacccacaaaatcagagggttatctgtacacaccccaggcttcctctgctgaagtagagatgagtgcctatgtcgtcttagctcgcctcactgcccagccagccccatcccctgaggacctggctttgtcaatgggcaccatcaagtggctcacaaagcagcagaattcccatggcggcttctcctccacacaggacactgtggtggcccttgatgctctgtccaaatacggagcagctactttttccaaaagccagaaaactcctttggtgaccattcaatcttcagggtcattttcccaaaagttccaagtagacaacagtaatcgcctgttactgcagcaggtctcattaccagacattcctgggaactataccgtcagtgtgtcaggggaaggatgtgtgtatgctcagaccacgctgagatacaacatgcccttggagaagcagcagcctgcatttgctctaaaggtgcagacagtacccctgacttgtaacaaccccaaaggccagaacagcttccagatctcactagaaatcagttacacggggagccgtccagcctccaacatggtgattgctgatgtgaagatgctctctggcttcatcccattgaaaccaacagtgaagaagcttgaaagattagagcacgtaagcagaacagaagtgaccaccaacaacgtcctgttatatttggaccaggtaaccaatcagacactgtccttctccttcatcattcaacaagacatcccagtaaagaacctgcagcctgccattgtgaaagtgtatgactactatgagacagatgaagtggcttttgctgaatacagcagcccctgcagctcagacaaacaaaatgtttgaaactcatatctgtggacaaggcattgctggaatccccaaaccagagaactttcaaggtggtgatttgtgtttgtctctgaaatacaaatactgaacaatcttggt

SEQ ID NO: 72 - DNA, 1852 bp, Homo sapiens
  misc_feature (1)..(1852): cDNA for actin, beta
accgccgagaccgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggaggcagccagggcttacctgtacactgacttgagaccagttgaataaaagtgcacaccttaaaaatgaaaaaaaaaaaaaaaaaaaaaaaaaaaaaaaaaaaaaaaaa

SEQ ID NO: 73 - DNA, 1731 bp, Rattus norvegicus
  misc_feature (1)..(1731): cDNA for cytochrome P450, family 2, subfamily c, polypeptide 7
ggcatcagtaaggagggtctccatggatctggtcactttcctggtacttactctctcctctctcattctcctctcactctggagacagagctctaggagaaggaagctccctcctggccccactcctctcccaataattggtaatttcctccagatagatgtgaagaacatcagccaatccttaaccaagttttcaaaaacctatggccctgtgttcactctgtatttgggctcacagcccactgtcatattgcatggatatgaagcaataaaggaagctctgattgataacggggagaagttttctggtagaggaagctatccaatgaatgaaaatgttactaaaggttttggcattgtttttagcaatggaaacagatggaaagagatgagacgattcacaatcatgaactttcggaatttgggaattggaaaaaggaacattgaggaccgtgttcaagaggaagcacagtgcctagtggaagaactgaggaaaacaaaaggctcaccctgtgacccctccttgatcctgaactgtgctccatgcaatgtcatctgctccattactttccagaatcattttgattataaagataaggaaatgcttacgttcatggaaaaagtgaatgagaatctcaagattatgagctccccatggatgcaggtctgcaatagttttccttcactcattgactattttcctggaactcatcacaaaatagcaaaaaatattaattatatgaaaagctatttgttgaagaaaatagaagaacaccaggaatcattggatgttacaaaccctcgtgattttgttgattattacctgattaaacaaaaacaggcaaacaacatcgaacaatcagaatattcacatgaaaatctgacatgcagtatcatggatctcattggtgcagggacagagacaatgagcacaacattgagatatgctctcctgcttctgatgaagtacccacatgtcacagctaaagtccaggaagagattgaccgtgtgattggcagacatcgcagcccctgcatgcaggatagaaaacacatgccctacactgatgccatgattcatgaggtacagagattcattaactttgtcccgaccaacctgccccatgcagtgacctgtgacattaaattcaggaactacctcatcccgaagggaacaaaagtgttaacatcactgacatcagtgctgcatgacagcaaggagttccccaacccagagatgtttgaccctggccactttctagatgagaatggaaactttaagaaaagtgactactttttgcctttctcagcaggaaaacgagcttgtgttggagagggcctggcccgcatgcagttgtttctattcttgacaaccattttacagaactttaacctgaaatctctggttcacccaaaggacattgatacgatgccagttctgaatggttttgcctctctgccacccacttaccagctctgcttcattccttcctgaatagatcaggcattttggcttctactgtgttgccttctgcaatccccttcaggtttttatccagtcccttccctctatcatccatcctgactcttgtcttaagatccagaaagcacatgcagtttctggagttatagcacaaatccacttgtattatctctttcatataacaatttatgatcccttgtgtcattgtgtatccattcctgagttaaaataaagcttcactataaaat

SEQ ID NO: 74 - DNA, 1307 bp, Rattus norvegicus
  misc_feature (1)..(1307): cDNA for glyceraldehyde-3-phosphate dehydrogenase
gggggctctctgctcctccctgttctagagacagccgcatcttcttgtgcagtgccagcctcgtctcatagacaagatggtgaaggtcggtgtgaacggatttggccgtatcggacgcctggttaccagggctgccttctcttgtgacaaagtggacattgttgccatcaacgaccccttcattgacctcaactacatggtctacatgttccagtatgactctacccacggcaagttcaacggcacagtcaaggctgagaatgggaagctggtcatcaacgggaaacccatcaccatcttccaggagcgagatcccgctaacatcaaatggggtgatgctggtgctgagtatgtcgtggagtctactggcgtcttcaccaccatggagaaggctggggctcacctgaagggtggggccaaaagggtcatcatctccgccccttccgctgatgcccccatgtttgtgatgggtgtgaaccacgagaaatatgacaactccctcaagattgtcagcaatgcatcctgcaccaccaactgcttagcccccctggccaaggtcatccatgacaactttggcatcgtggaagggctcatgaccacagtccatgccatcactgccactcagaagactgtggatggcccctctggaaagctgtggcgtgatggccgtggggcagcccagaacatcatccctgcatccactggtgctgccaaggctgtgggcaaggtcatcccagagctgaacgggaagctcactggcatggccttccgtgttcctacccccaatgtatccgttgtggatctgacatgccgcctggagaaacctgccaagtatgatgacatcaagaaggtggtgaagcaggcggccgagggcccactaaagggcatcctgggctacactgaggaccaggttgtctcctgtgacttcaacagcaactcccattcttccacctttgatgctggggctggcattgctctcaatgacaactttgtgaagctcatttcctggtatgacaatgaatatggctacagcaacagggtggtggacctcatggcctacatggcctccaaggagtaagaaaccctggaccacccagcccagcaaggatactgagagcaagagagaggccctcagttgctgaggagtccccatcccaactcagcccccaacactgagcatctccctcacaattccatcccagaccccataacaacaggaggggcctggggagccctcccttctctcgaataccatcaataaagttcgctgcaccctcaaaaaaaaaaaaaaaaaaaaaaaaaaaaaa

SEQ ID NO: 75 - DNA, 960 bp, Rattus norvegicus
  misc_feature (1)..(960): cDNA for alpha-2u globulin PGCL4
tgggcaccatcagcagagagattgtcccgacagagaggcaattctattccctaccaacatgaagctgttgctgctgctgctgtgtctgggcctgaccctggtctgtggccatgcagaagaagctagtttcgagagagggaacctcgatgtggacaagctcaatggggattggttttctattgtcgtggcctctgataaaagagaaaagatagaagagaacggcagcatgagagtttttgtgcagcatatcgatgtcttggagaattccttaggcttcacgttccgtattaaggaaaatggagtgtgcacagaattttctttggttgccgacaaaacagcaaaggatggcgaatattttgttgagtatgacggagagaatacatttactatacttaagacagactatgacaattatgtcatgtttcatctcgttaatgtcaacaacggggaaaccttccagctgatggagctctatggcagaacaaaggatctgagttcagacatcaaggaaaagtttgcaaaactatgtgtggcacatggaatcactagggacaatatcattgacctaaccaagactgatcgctgtctccaggcctgaggttgaagaaaggcctgagcctccagagtgcctctctgtccagaagtcaatccaagaagtgcttaatgggttcctttattctttcttcctggattactccatgctgagtggagacttctcaccaggactccagcattaccatttcctgtccatggagcatcctgagacaaattctgcgatctgatttccatcctgtctcacagaaaagtgcaatcccggtctctccagcatcttccctagttacccaggacaacacatcgagaattaaaagctttcttaaatttctctttgccccacccatgatcattccgcacaaatttcttgctcttgcagtgcaataaatgattacccttgcacttaaa

SEQ ID NO: 76 - DNA, 1310 bp, Homo sapiens
  misc_feature (1)..(1310): cDNA for glyceraldehyde-3-phosphate dehydrogenase
aaattgagcccgcagcctcccgcttcgctctctgctcctcctgttcgacagtcagccgcatcttcttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaacc

SEQ ID NO: 77 - DNA, 1554 bp, Rattus norvegicus
  misc_feature (1)..(1554): cDNA for fibrinogen gamma chain
ctgggagcaccggtcacccagacactatgaattggtccttgcaactccggagtttcattctatgctgggcgcttttactactttctccaacgggcctggcacagtacactgctactagagataactgttgcatcctagatgagagattcggtagttactgcccaaccacttgtggcatctcagacttcctgaattcttaccaaaccgacgtggacactgacctccagactctggaaaacatcttacaacgagctgaaaacagaaccacagaagccaaggaactgattaaagccatccaggtttactacaacccagaccaacccccaaagccgggtatgatagagggtgccactcagaagtctaagaagatggtagaagaaatcttgaagtacgaagcactgttactaacccatgagtcgagtatccggtatttacaggacatctatacttcaaataagcagaagatcacgaacctgaaacagaaggtggcccagcttgaagctcagtgccaggagccttgcaaggactctgtgcgaatccatgacacaaccggaaaagattgtcaggatatcgccaacaagggtgccaaagaaagtggactttacttcattcggcctttgaaagccacgcagcagttcttagtgtactgtgaaatcgacggatctggaaatggctggaccgtattgcagaagaggcttgatggcagtgtggatttcaagaagaactggattcagtataaagaaggatttggacacctgtcacctactggcaccacagagttttggctgggcaatgagaagattcatctgataagcatgcagtccaccatcccatacgcactgagaatacagctcaaagactggagtggcaggaccagcaccgcggactatgccatgttcagggtgggtcctgaatccgacaaataccgcctgacctatgcctacttcattggcggagatgccggggatgccttcgacggctacgattttggtgatgatcccagtgacaagtttttcacatcccacaacggcatgcacttcagtacctgggacaatgacaacgacaagttcgaaggcaactgtgctgagcaggatggatctggctggtggatgaacaagtgtcacgctggccacctcaatggagtttattaccaaggtggcacttactccaagtcatctactcctaacggttatgacaatggcattatttgggccacctggaaaacccgctggtattccatgaaggaaaccaccatgaagataattcccttcaacagactctccattggagatgggcagcaacatcacatgggaggatccaaacaggttggagacatgtaaagaacatttccaaggtgatttgctcttttaacggactttatccaagcagagagatatatttttcctatgagaccatggaacctcacttcctttacagagttaatgggatccatgatgcaaactccattagcagttttatgctggcgataatttatctacatgcatttcaataaacattttgtttcctaaaaaaaaaaaaaaaaaaaaaaaaaaa

SEQ ID NO: 78 - DNA, 1665 bp, Homo sapiens
  misc_feature (1)..(1665): cDNA for fibrinogen gamma chain
cttctggtaaggaggccccgtgatcagctccagccatttgcagtcctggctatcccaggagcttacataaagggacaattggagcctgagaggtgacagtgctgacactacaaggctcggagctccgggcactcagacatcatgagttggtccttgcacccccggaatttaattctctacttctatgctcttttatttctctcttcaacatgtgtagcatatgttgctaccagagacaactgctgcatcttagatgaaagattcggtagttattgtccaactacctgtggcattgcagatttcctgtctacttatcaaaccaaagtagacaaggatctacagtctttggaagacatcttacatcaagttgaaaacaaaacatcagaagtcaaacagctgataaaagcaatccaactcacttataatcctgatgaatcatcaaaaccaaatatgatagacgctgctactttgaagtccaggaaaatgttagaagaaattatgaaatatgaagcatcgattttaacacatgactcaagtattcgatatttgcaggaaatatataattcaaataatcaaaagattgttaacctgaaagagaaggtagcccagcttgaagcacagtgccaggaaccttgcaaagacacggtgcaaatccatgatatcactgggaaagattgtcaagacattgccaataagggagctaaacagagcgggctttactttattaaacctctgaaagctaaccagcaattcttagtctactgtgaaatcgatgggtctggaaatggatggactgtgtttcagaagagacttgatggcagtgtagatttcaagaaaaactggattcaatataaagaaggatttggacatctgtctcctactggcacaacagaattttggctgggaaatgagaagattcatttgataagcacacagtctgccatcccatatgcattaagagtggaactggaagactggaatggcagaaccagtactgcagactatgccatgttcaaggtgggacctgaagctgacaagtaccgcctaacatatgcctacttcgctggtggggatgctggagatgcctttgatggctttgattttggcgatgatcctagtgacaagtttttcacatcccataatggcatgcagttcagtacctgggacaatgacaatgataagtttgaaggcaactgtgctgaacaggatggatctggttggtggatgaacaagtgtcacgctggccatctcaatggagtttattaccaaggtggcacttactcaaaagcatctactcctaatggttatgataatggcattatttgggccacttggaaaacccggtggtattccatgaagaaaaccactatgaagataatcccattcaacagactcacaattggagaaggacagcaacaccacctggggggagccaaacaggctggagacgtttaaaagaccgtttcaaaagagatttacttttttaaaggactttatctgaacagagagatataatatttttcctattggacaatggacttgcaaagcttcacttcattttaagagcaaaagaccccatgttgaaaactccataacagttttatgctgatgataatttatctacatgcatttcaataaaccttttgtttcctaagactagaaaaa

SEQ ID NO: 79 - DNA, 595 bp, Rattus norvegicus
  misc_feature (1)..(595): cDNA for transthyretin
cctgacaggatggcttcccttcgcctgttcctcctctgcctcgctggactgatatttgcgtctgaagctggccctgggggtgctggagaatccaagtgtcctctgatggtcaaagtcctggatgctgtccgaggcagccctgctgtcgatgtggccgtgaaagtgttcaaaaggactgcagacggaagctgggagccgtttgcctctgggaagaccgccgagtctggagagctgcacgggctcaccacagatgagaagttcacggaaggggtgtacagggtagaactggacaccaaatcatactggaaggctcttggcatttccccattccatgaatacgcagaggtggttttcacagccaatgactctggtcatcgccactacaccatcgcagccctgctcagcccgtactcctacagcaccactgctgtcgtcagtaacccccagaactgagggacccagcccacgaggaccaagatcttgccaaagcagtagctcccatttgtactgaaacagtgttcttgctctataaaccgtgttagcaactcgggaagatgccgtgaaacgttcttattaaaccacctttatttcattc

SEQ ID NO: 80 - DNA, 938 bp, Homo sapiens
  misc_feature (1)..(938): cDNA for transthyretin
gttgactaagtcaataatcagaatcagcaggtttgcagtcagattggcagggataagcagcctagctcaggagaagtgagtataaaagccccaggctgggagcagccatcacagaagtccactcattcttggcaggatggcttctcatcgtctgctcctcctctgccttgctggactggtatttgtgtctgaggctggccctacgggcaccggtgaatccaagtgtcctctgatggtcaaagttctagatgctgtccgaggcagtcctgccatcaatgtggccgtgcatgtgttcagaaaggctgctgatgacacctgggagccatttgcctctgggaaaaccagtgagtctggagagctgcatgggctcacaactgaggaggaatttgtagaagggatatacaaagtggaaatagacaccaaatcttactggaaggcacttggcatctccccattccatgagcatgcagaggtggtattcacagccaacgactccggcccccgccgctacaccattgccgccctgctgagcccctactcctattccaccacggctgtcgtcaccaatcccaaggaatgagggacttctcctccagtggacctgaaggacgagggatgggatttcatgtaaccaagagtattccatttttactaaagcagtgttttcacctcatatgctatgttagaagtccaggcagagacaataaaacattcctgtgaaaggcacttttcattccactttaacttgattttttaaattcccttattgtcccttccaaaaaaaagagaatcaaaattttacaaagaatcaaaggaattctagaaagtatctgggcagaacgctaggagagatccaaatttccattgtcttgcaagcaaagcacgtattaaatatgatctgcagccattaaaaagacacattctgtaaaaaaaaaaaa

SEQ ID NO: 81 - DNA, 306 bp, Rattus norvegicus
  misc_feature (1)..(306): cDNA for apolipoprotein C-III
atgcagccccgaatgctcctcatcgtggccctcgtggctctcctggcctctgcccgagctgatgagggagagggatccttgctgctgggctctatgcagggctacatggaacaagcctccaagacggtccaggatgcactaagcagcatgcaggagtctgatatagctgtggtggccagcaggggctggatggacaatcgcttcaaatccctgaaaggctactggagcaagttcactgataagttcactggcctctgggagtctggccctgaggaccaactaacaacaccaactcttgagccgtga

SEQ ID NO: 82 - DNA, 533 bp, Homo sapiens
  misc_feature (1)..(533): cDNA for apolipoprotein C-III
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 83 - DNA, 1628 bp, Rattus norvegicus
  misc_feature (1)..(1628): cDNA for vitronectin
gattctttccccaggcaagacagtgagactccagaagctccgagcccagtttgccaaggccctctgctagaggcctgccatggcatctctgaggccctttttcatactagccctgctggcattggtttctctggctgaccaagagtcatgcaagggccgctgcactcagggtttcatggccagcaagaagtgtcaatgtgatgagctttgcacctactatcagagctgctgtgttgactacatggagcagtgcaagccccaagtgacccggggggatgtgtttaccatgccagaagatgagtattggagctatgactacccggaggaaaccaagaacagtaccagcactggcgtgcaatccgagaacacctctctacacttcaacctaaagcctcgagccgaagagactataaagccaacaactcctgatcctcaggaacagtcaaacacccaagaacctgaagtggggcaacagggagtggccccaaggcctgacaccactgatgaaggaacctctgagtttccagaagaagagctgtgtagtggaaagccctttgatgccttcacagacctcaagaatggttccctttttgccttccgaggtgagtattgctatgagctagatgaaacagcagtgaggcctgggtaccccaaactcatccaagatgtctggggcatcgagggccccatcgacgctgccttcactcgcatcaactgtcaggggaagacctacttgttcaagggtagtcagtactggcgctttgaggatggtgtcctggaccctgattatccccgaaacatctctgaaggcttcagtggcataccagacaacgttgacgcagccttagcccttcctgcccacagttacagtggccgggaaagggtctacttcttcaagggaaaacagtactgggagtatgaatttcagcagcagcccagccaggaggaatgtgaaggaagttctctgtcagccgtgtttgagcactttgccttgcttcagcgggacagctgggagaacatttttgaactcctcttctggggcagatcctctgatggcgccaaaggaccccaattcatcagcagggattggcatggtgtaccagggaaagtggacgctgctatggccggccgcatctacatcactggctccacattccgctctgtccaggccaaaaaacagaagtctgggcgtcgaagtcgaaaacgctaccgttcacgccgaggacgtggccatagccgcagccgcagtcgcagcatgagctctcgtcgcccatcgcgttcagtctggttctctttgctctccagcgaagagagtgggctaggaacctacaattacgattatgatatgaactggcgcatacctgccacctgcgagcccattcagagcgtctatttcttctctggagacaagtactaccgagttaaccttagaacacggcgagtggactctgtgaatcctccctacccacgctccattgctcagtattggctgggctgcccaacctctgagaagtaggaatcagcacccacacagctgagcttcagaaacctcgtcctccagctttttcctcccagcccaataaagtgttggccccaaaaaaaaaaaaaaaaaaaaaaaaaaaaaaaaa

SEQ ID NO: 84 - DNA, 1678 bp, Homo sapiens
  misc_feature (1)..(1678): cDNA for vitronectin
gagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtaggagtcagagcccacatggccgggccctctgtagctccctcctcccatctccttcccccagcccaataaaggtcccttagccccgagtttaaa

SEQ ID NO: 85 - DNA, 889 bp, Rattus norvegicus
  misc_feature (1)..(889): cDNA for apolipoprotein A-I
agactgttggagagctccgggggaggtcgccacatccttcaggatgaaagctgcagtgttggctgtggccctggtcttcctgacaggttgccaagcttgggagttctggcagcaagatgagccccagtcccaatgggacagggtgaaggatttcgccactgtgtatgtggatgcagtcaaggacagcggcagagactatgtgtcccagtttgaatcctccactttgggcaaacagctgaacctgaatctcctggacaactgggacactctgggttcaactgttggtcgcctacaggaacagctaggcccagtgactcaggagttctgggctaacctggagaaagaaacagattggctgagaaacgagatgaacaaggacctggagaatgtgaaacagaagatgcagccccacctggatgaattccaggagaagtggaacgaggaggtcgaggcctaccgccagaagctggagcctctgggcaccgagcttcacaaaaacgcgaaggagatgcaaaggcatctaaaggttgtggccgaggagtttcgagaccgcatgcgtgtgaatgcagacgcgctgcgcgcaaagtttgggctctacagcgatcagatgcgcgagaacctggcccagcgcctgaccgagatcaagaaccaccctaccctgatcgagtatcataccaaggccagcgaccacctgaagacacttggtgagaaagccaaacccgcgctggatgacctgggccagggcctgatgccggtgctggaagcctggaaggccaaaatcatgagtatgatcgatgaggccaaaaagaagctgaacgcttagtgaggcgcccgtcaccactccccacccctgaattggctttcttacaataaacgtttccaaagtggg

SEQ ID NO: 86 - DNA, 897 bp, Homo sapiens
  misc_feature (1)..(897): cDNA for apolipoprotein A-I
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 87 - DNA, 2906 bp, Rattus norvegicus
  misc_feature (1)..(2906): cDNA for inter-alpha trypsin inhibitor, heavy chain 1
gcctggagaccatggatggtgtcacaggactacgggtgctgctgtgtctcctcactctgcagaccatgcctgctctgggcttggcaacaggcagacccaggggcaatgagaagcgacaggctgtggacacaacagtcaatggtgtgtccatcaggagcttgaaagtcaattgcaaagtcacctctcgctttgcccactacgtcatcaccagtcaggtggtcaacagtgctaacaaagccagggaagtggcttttgatgtggaaatccccaagacagccttcatcagtgactttgccataacagcagatgggaatgcattcattggggacataaaggacaaagtgactgcatggaagcaataccggaaggcagccatttcaggagagaatgctggccttgtcagggcctcgggaagaaatatggagcagtttaccatccacatcactgttggagcccagagcaaggccacgttccagctcacctatgaggaggtactaaagaggagacttacgcagtatgacatcgtcatcaaagtgagaccgaagcagctggtacaccattttgagattgatgtggacatctttgagccgcaggggatcagcaggctggatgctcaagcctccttcctcagcaaggaactggctgctcaaaccatcaagaagtctttctcggggaagaagggtcatgtgctcttccgccccactgtgagccagcagcagtcctgccccacgtgctctacctccttgctgaatggagacttcaaggtgacctacgatgtgaaccgagacaagctctgtgacctcctggtggccaacaattactttacacatttctttgccccccaaaacctgaccaacatgagcaagaacctggtttttgtgattgatatcagtgggtccatggaaggccagaaagtgaagcagacaaaggaggcgctccttaagatattgggggacatgaagccagtagacaactttgacctggtcctctttgggtctcaagtgcagtcatggaagggctcactggtacccgcgtctcacgccaatttgcaagcagcccaagactttgtgaggcgcttttctttggctggagccacaaacctgaatggaggcttgctccgaggaatcgagatcttaaacagagctcaaggaagccacccagaactcagcagccctgcctcaattcttatcatgttgaccgacggagagcccactgaaggggagacggaccgttcccagatcctcaagaatgtccgcaacgctatccggggcagattcccgctctacaaccttggctttggtcacgacctggactttagcttcctggaggtcatgtccattgagaacaacggatgggcccagagaatttacgaggaccacgatgccacccaacagctacagggtttctacaatcaagtagcccaccccctgctgacagatgtggagctgcagtacccccaggacgctgtcttggctctaacacagcaccgacacaaacagtactatgatggctctgagatcgtggtggctggacgcattgctgaccacaaactgagcacctttaaggcggatgttcgggctcatggggaaaagcaagaattcaaggcaacctgcctggtggacgaggaagagatgaagaagctgctccgagagcgtggccacatgctagagaatcatgtggagaggctctgggcctacctcactatccaggagctcctggctaagaggatgaagacagaaggggaagagagggccaacctgtcgtcccagatcttgaagatgtcactggactatcatttcgtaacgccgctgacctctatgactatcagaggcctgacagacgaggatgggctggagcccatcattgacaagcccccagaggattctcagcccttagagatggtgggacccagaagaacgtttgtgctgtcagccgcacagccttctcctacagcccaccccataggctcaaagttgccaacccgtgtgacaggtgtggataccgatccccacttcatcatctatgtacccacaaaagaggatagcctgtgcttcaacatcaatgaggaacccggtgtgatcctgaacctagtgcaggaccctgacacaggattctcggtgaatgggcagctcattgggataaaggccggcagccctgggcagcatgagagcacatactttgggagactgggggtctcaagccccacatcagactttcagctggaagtgactcctcagaacattacactgaaccccagctctggcgggccggtgttctcctggagggatcaggctgtgctgcagaaggacggggtagtggtgaccatcaacaagaagaggaacctggtggtgtctgtggacgacggggctacctttgagattgtcctccacagaacatggagggggagttcaatccaccaggacttcctgggtttctatgtgctggatagcttccggatgtcagcccggacacagggactattggggcaattcttctccccactggattttgaagtatttgacctccgcccaggctctgaccctacaaagacagatgccacaatggtggttaagaatcggcagctgacagtcacccgagggttacaaaaggactacagcaaggaccccagacatggaacagaagtgtcttgctggtttgtccacgacaatggagctggactgattgatggcgtccacactgactatatcgtctctgacatcttctgagccctccagctagcacagcagcccttgcctggaactgcatgtgaagtgggagggacgagcttagcaccagattagctccaccttcctgagcaaactggccctttgtttcaagatatcctgtgatttcattaaagagaagctgtgtccaaaaaaaaaaaaaaaaaaaaaaaaaaaaaaaaa

SEQ ID NO: 88 - DNA, 2741 bp, Homo sapiens
  misc_feature (1)..(2741): cDNA for inter-alpha (globulin) inhibitor H1
gtgcgtgcctgcccaactcccatgccttctcccaggggtgccgcttactagtcccagcttaagaacaaggatcggagtggccagataacgcagagcatctcctcattctagaagggccacagaccagccctggctttgaggggaggaagaagccctttagtacagcagatggaaacgcatttatcggagacataaaggacaaggtgactgcatggaagcagtaccggaaagcagctatctcaggagagaatgccggccttgtcagggcctcggggagaactatggagcaattcaccatccacctcaccgtcaatccccagagcaaggtcacgtttcagctgacttatgaggaagtgctgaagagaaaccatatgcagtatgaaattgtcatcaaagtcaagcccaagcagctggtgcatcattttgagattgatgtggacatcttcgagccccaggggatcagcaagctggatgcccaggcctctttcctgccgaaggaactggcagcccaaactatcaagaagtccttctcaggaaaaaagggtcatgtgctgttccgtcccaccgtgagccagcagcagtcctgccccacatgctctacatccttactgaacgggcacttcaaggtgacctacgatgtcagtcgagacaagatctgcgacctcctggtggccaataaccactttgcccacttctttgccccccaaaacctgacaaacatgaacaagaacgtggtttttgtgattgacatcagtggctccatgagaggccagaaagtgaagcagaccaaggaggcactccttaaaattctgggggacatgcagccaggggactactttgacctggttctttttgggactcgagtacaatcgtggaagggctcgctggtgcaagcatctgaggccaacctacaagcagctcaagactttgtgcggggcttttccctggatgaggccacaaacctgaatggaggtttgctccggggaattgagatcttgaaccaagttcaggaaagcctcccagaactcagcaaccatgcctcaatactcatcatgttgacagatggcgatcccacagagggggtgacggaccgttcccaaatcctcaagaacgtccgcaacgccatccggggcaggttcccgctctacaacctgggtttcggccacaatgtggactttaactttctggaggtcatgtccatggagaacaacggacgggcccagagaatctacgaggaccatgatgccacccagcagctgcagggtttctacagccaggtagccaaacccctgctggtggatgtggatttgcagtacccccaggatgctgtcttggccctgacccagaaccaccataaacagtactacgaaggctcagagattgtggtggccgggcgcattgctgacaacaaacagagcagcttcaaggctgatgtgcaggcccatggggagggacaagaattcagtataacctgcctagtggatgaggaggagatgaagaaactgctccgagagcgtggccacatgctggagaaccacgtcgagcgcctctgggcctacctcaccatccaggagctgctggccaagcggatgaaggtggacagggaggagagggccaacctgtcatcccaggccctgcagatgtcgctggactatgggtttgtgaccccactgacctccatgagcatcaggggcatggcggaccaggacggcctgaagcccaccatcgacaagccctcagaggattctccgcctttggagatgctgggacccagaaggacgttcgtgctgtcagccttgcagccttctcctactcattccagctccaatacccagcggctgccagaccgagtgaccggcgtggacacagaccctcacttcatcatccacgtgccccagaaagaggacaccctgtgcttcaacatcaatgaggagcctggtgttatcctgagcctggtacaggaccccaacacaggcttctcagtgaatggacagctcattggcaacaaggccaggagccctgggcagcatgacggcacgtacttcgggcggctgggaatcgcaaaccctgccacggactttcagttggaagtgactcctcagaacattacgctgaaccccggctttggtgggcctgtgttttcctggagggaccaagctgtgctgcggcaggacggggtggtggtgaccatcaacaagaagaggaacctggtggtgtctgtggacgacggtggcacctttgaggttgttttgcaccgagtgtggaaggggagctcggtccaccaggacttcctgggcttctatgtgctggacagtcatcggatgtcagcccggacgcacgggctgctggggcaatttttccaccccatcggttttgaagtgtctgacatccacccaggctctgaccccacaaagccagatgccacgatggtggtgaggaaccgccggctcacggtcaccaggggtttgcaaaaagactacagcaaggacccgtggcatggggccgaggtgtcctgctggttcattcacaacaatggggctggactcatcgatggtgcctacactgattatatcgtccccgacatcttctgagccctctggccagcacgcctgtcctcccccggggccaaggcagaggaggaggacgacatcctgacctgctgctgaggctgtacctccttgactaagctggttccttgtgtcaaagcacctcatgccttccattaaagagaggccgtgtcc

SEQ ID NO: 89 - DNA, 1141 bp, Rattus norvegicus
  misc_feature (1)..(1141): cDNA for fibrinogen-like 1
ggccattacggccgggggacttcagttagaagttcctgggaggccctgtgtagacccagcctagctgagtactgattcattttgatgtgagtgggaagaatgggggagattcgcagctttgtcctcatcactgttgctctgattctgggcaaggagagctgggtcctcggagatgagaactgtttgcaggagcaggtgaggctcagggctcaggtgcgccagcttgagacccgggtcaaacaacaacaggtggtgattgcacagctcttgcacgagaaggaggtccagttcctggatagaggacaggaggacagcttcattgaccttggaggcaagaggcattacgcagattgttcagagatttacaatgatggatttaaacatagtgggttttacaaaatcaaacctcttcagagtctggcagaattctctgtttattgtgatatgtctgatggaggaggatggactgtaattcagagacgatctgacggcagtgagaactttaacaggggttggaacgactatgaaaatggctttggaaactttgtccaaagcaatggtgaatactggctgggtaacaaaaacattaacttgctgactatgcaaggagactacactttaaaaatcgacctgacagactttgagaaaaacagccgcttcgcacaatacgaaaaatttaaagttggcgatgaaaagtctttttacgaactgaatattggagaatattctggcaccgccggagactccctgtcgggaacatttcaccctgaagtgcagtggtgggctagtcaccaaacaatgaagttcagcacacgcgacagagacaacgacaactacaacgggaactgtgctgaggaggaacagtctggctggtggttaacaggtgtcactctgcaaacctgaacggcgtgtactaccaaggtccctacagagcagaaaccgataatggtgttgtntggtacacctggcgtgggtggtggtattccttgaaatctgtggttatgaaaattaggcccagtgattttattccaaatatcgtttagttgtcccattgggatctgctttctgtgattcatcttggtttttaaatgtttgaaaaaaatatacaattgtgaaaaaaaaaaaaaaaaaaaaaaaaaaa

SEQ ID NO: 90 - DNA, 1285 bp, Homo sapiens
  misc_feature (1)..(1285): cDNA for fibrinogen-like 1
aattacaaattcccaatgcagttacaggatcctgggaagcagagtgtctggatggaacctgagctgggtctctgactcacttctgactttagttttttcaagggggaacatggcaaaggtgttcagtttcatccttgttaccaccgctctgacaatgggcagggaaatttcggcgctcgaggactgtgcccaggagcagatgcggctcagagcccaggtgcgcctgcttgagacccgggtcaaacagcaacaggtcaagatcaagcagcttttgcaggagaatgaagtccagttccttgataaaggagatgagaatactgtcattgatcttggaagcaagaggcagtatgcagattgttcagagattttcaatgatgggtataagctcagtggattttacaaaatcaaacctctccagagcccagcagaattttctgtttattgtgacatgtccgatggaggaggatggactgtaattcagagacgatctgatggcagtgaaaactttaacagaggatggaaagactatgaaaatggctttggaaattttgtccaaaaacatggtgaatattggctgggcaataaaaatcttcacttcttgaccactcaagaagactacactttaaaaatcgaccttgcagattttgaaaaaaatagccgttatgcacaatataagaatttcaaagttggagatgaaaagaatttctacgagttgaatattggggaatattctggaacagctggagattcccttgcggggaattttcatcctgaggtgcagtggtgggctagtcaccaaagaatgaaattcagcacgtgggacagagatcatgacaactatgaagggaactgcgcagaagaagatcagtctggctggtggtttaacaggtgtcactctgcaaacctgaatggtgtatactacagcggcccctacacggctaaaacagacaatgggattgtctggtacacctggcatgggtggtggtattctctgaaatctgtggttatgaaaattaggccaaatgattttattccaaatgtaatttaattgctgctgttgggctttcgtttctgcaattcagctttgtttaaagtgatttgaaaaatactcattctgaacatatccatgcgcaatcatgataactgttgtgagtagtgcttttcattcttctcacttgcctttgttacttaatgtgctttcagtacagcagatatgcaatattcaccaaataaatgtagactgtgttaaaaaaaaaaaaaaaaaaaaaaaaaaaaaaaaaaaaaa

SEQ ID NO: 91 - DNA, 1519 bp, Rattus norvegicus
  misc_feature (1)..(1519): cDNA for kininogen 1-like 1
agtcagagactggaggctgctaaaagcagcttctgagttggtttccctttttggcaaaacaattcctcaattccagagggaacctttaatcatgaagctaattactatcctgctcctctgctccaggctcctgccaagtttagcgcaggaagaaggtgcccaggaaatggactgcaatgatgagactgtatttcaggctgtggatactgctctgaagaaatataatgctgagttagaaagtggcaaccagtttctgttgtaccgagtgactgagggcactaagaaggatggcgctgaaacattgtattccttcaagtatcaaatcaaggagggcaactgctctgttcagagtggcctcacctggcaggactgtgacttcaaggacgctgaggaagctgctactggcgaatgcacaacaactttggggaagaaagaaaataaattctccgtagccacccagatctgcaatattactccaggtaagggtcctaagaagacagaggaggacctctgtgtcgggtgtttccaacccataccgatggatagctcagacctgaagcctgttctgaaacacgctgtggagcatttcaacaacaacacgaagcacacccacctctttgctctcacagaagtaaagagtgcccactcacaggtggtggctggcatgaattataaaattatctactccattgtgcaaacaaattgttcaaaggaggattttcctttcctccgtgaagactgtgtaccccttccctatggcgatcatggtgagtgtaggggtcatacctacgtggatattcataacacaattgctggcttctcacagagctgtgacctttatccaggagacgatttgttttcactacttcccaagaaatgctttggctgccccaagaacatacctgtagacagcccggagctcaaggaggcacttggtcattcaattgcacagcttaatgcacagcataatcatcttttctatttcaagattgacaccgtgaaaaaggcaacatcacaggtggttgctggaacaaaatatgtgattgagttcatagccagagaaaccaattgttccaaacaaactaacacagaactgacagcggactgtgagaccaaacacctcggtcaaagcctcaactgcaatgctaacgtgtacatgagaccttgggagaacaaagtcgtcccgactgtcagatgccaagcgctagacatgatgatttctaggcctccaggattttcacctttccggctggtgcaagtacaagaaactaaagaaggaacaactaggctcctaaactcatgtgagtacaagggcagactctcaaaggcaggggcaggcccagcgcctgaccatcaggcagaagcttcaaccgtgacaccatagcctggcaaagacccggagtggaaggaccagaagactcctgggatgtgtgcagtatggaagcatgtttcttcatcacctgatcctgggtgaaataaagttcagactcgacgaaaaaaaaaaaaaaaaaaaaaaaaa

SEQ ID NO: 92 - DNA, 2143 bp, Homo sapiens
  misc_feature (1)..(2143): cDNA for kininogen 1
agtgttgcaatgttctagctgtaaatatgtttggataacccagcagagccatttaacctttgggaacaaggcaactagcgtctggcagcaggaatccaaccagtgccctgagttctgaggcagagaggaggacagaagaaacaagaggctggagattgtcaaattcagtatcccagttggctcttgattcttggtgaaaccatccctcagctcctagagggagattgttagatcatgaaactaattaccatccttttcctctgctccaggctgctactaagtttaacccaggaatcacagtccgaggaaattgactgcaatgacaaggatttatttaaagctgtggatgctgctctgaagaaatataacagtcaaaaccaaagtaacaaccagtttgtattgtaccgcataactgaagccactaagacggttggctctgacacgttttattccttcaagtacgaaatcaaggagggggattgtcctgttcaaagtggcaaaacctggcaggactgtgagtacaaggatgctgcaaaagcagccactggagaatgcacggcaaccgtggggaagaggagcagtacgaaattctccgtggctacccagacctgccagattactccagccgagggccctgtggtgacagcccagtacgactgcctcggctgtgtgcatcctatatcaacgcagagcccagacctggagcccattctgagacacggcattcagtactttaacaacaacactcaacattcctccctcttcatgcttaatgaagtaaaacgggcccaaagacaggtggtggctggattgaactttcgaattacctactcaattgtgcaaacgaattgttccaaagagaattttctgttcttaactccagactgcaagtccctttggaatggtgataccggtgaatgtacagataatgcatacatcgatattcagctacgaattgcttccttctcacagaactgtgacatttatccagggaaggattttgtacaaccacctaccaagatttgcgtgggctgccccagagatatacccaccaacagcccagagctggaggagacactgactcacaccatcacaaagcttaatgcagagaataacgcaactttctatttcaagattgacaatgtgaaaaaagcaagagtacaggtggtggctggcaagaaatattttattgacttcgtggccagggaaaccacatgttccaaggaaagtaatgaagagttgaccgaaagctgtgagaccaaaaaacttggccaaagcctagattgcaacgctgaagtttatgtggtaccctgggagaaaaaaatttaccctactgtcaactgtcaaccactgggaatgatctcactgatgaaaaggcctccaggtttttcacctttccgatcatcacgaataggggaaataaaagaagaaacaactagtcacctaaggtcctgcgagtacaagggtcgacccccaaaggcaggggcagagccagcatctgagagggaggtctcttgaccaatgggcagaatcttcactccaggcacatagccccaaccacctctgccagcaaccttgagaggaaggacaagaagaaagatgggatagaatttaaatagagaagaatgccattttatcactctgcctctgggtgaaataaagatcagtcttgatgttctaactctaattcacagtggtctcctttcagccctacccattctgcagcaaattccagctggtcagagagtcagtgctgtggctctgccatggaggctcataacccaacactggaacattccctagccaaggcagaagtccttaggcgggacttccttaccaccacgggtgctaaaagaagagttagtaggtcatgcttctaccagtaatctaaggactctctccttctcttcttcctctttctccagatttccaagccttagctaagagtaatttggcttgtttagtattgttttcttatggtccagttaattaccaaaaatatttttaaaatcatctctgttaatagaatgtctaccaacttctcactatcagaaaatactcaacctccaaacaatttgaaattatctttctgatccaggtaaagaagcaaattgaaaatcaataaagtaaaaagtgacggaga

SEQ ID NO: 93 - DNA, 1431 bp, Rattus norvegicus
  misc_feature (1)..(1431): cDNA for group specific component
atgaagagggttctggttctcctgctggccttagcctttgggcatgccctagagagaggccgagattatgagaaggataaagtttgccaggaactctccacgctggggaaagatgacttcagatctctatcactaatcctatacagcaggaagtttcccagtagcacctttgaacaggtcagtcagcttgtgaaggaagttgtctccttaactgaggaatgctgtgctgagggagcagatcccaactgctatgacaccaggacttcagagctgtctattaagtcctgtgaaagtgacgccccctttcctgttcaccccgggacttccgagtgttgcacgaaggaggggctagagcggaaactctgcatggctgccctcagtcaccagccacaggaatttcccgcctatgtggagccgacgaatgatgagatctgtgaggcgttcaggaaggatccaaagggatttgccgatcagtttctattcgaatattccagcaattacggacaagctcctctgccacttttagttggttacaccaagagttatctctctatggtcggctcctgctgtacttctgcaaaaccaactgtgtgctttttgaaggagagactccagatgaaacagttactacttctcaccactatgtcaaacagagtctgctcacaatatgctgcatatggaaaggaaaaatcaaggatgagccatctcataaaactagcccaaaaagtaccaactgctaacctggaggatgttctgccactcgctgaagaccttactgaaatcctgtccagatgttgtaagtctacctcagaggactgcatggccagagagctgcctgaacacacattaaaaatctgtggaaacttatccaaaaagaattcaaagtttgaagagtgctgttacgaaacaacacccatgggcatttttatgtgtagctacttcatgccaactgctgaaccacttcaattgccagccatcaagctgccaaccagcaaagacctctgtggtcagagtgccacacaagccatggaccagtatacatttgaactaagcagaaggacccaggttccagaagtgttcctcagcaaagttctggatacgaccctgaaaacccttagggaatgctgcgacactcaggactctgtctcctgtttcagcactcagagtcccctgatgaagaggcaactaacttctttcattgaaaaggggcaagaaatgtgtgcagattattccgagaacacatttactgagtacaagaaaaaattggcggaacgactgaggacaaaaatgcccaatgcctccccggaagagctggcagacatggtggccaagcactcagactttgcctccaagtgctgctccataaactctcctccacgctactgtagctcacagattgatgcagaaatgagagacatcctgcagtcctga

SEQ ID NO: 94 - DNA, 1801 bp, Homo sapiens
  misc_feature (1)..(1801): cDNA for group-specific component
tttaataataattctgtgttgcttctgagattaataattgattaattcatagtcaggaatctttgtaaaaaggaaaccaattacttttggctaccacttttacatggtcacctacaggagagaggaggtgctgcaagactctctggtagaaaaatgaagagggtcctggtactactgcttgctgtggcatttggacatgctttagagagaggccgggattatgaaaagaataaagtctgcaaggaattctcccatctgggaaaggaggacttcacatctctgtcactagtcctgtacagtagaaaatttcccagtggcacgtttgaacaggtcagccaacttgtgaaggaagttgtctccttgaccgaagcctgctgtgcggaaggggctgaccctgactgctatgacaccaggacctcagcactgtctgccaagtcctgtgaaagtaattctccattccccgttcacccaggcactgctgagtgctgcaccaaagagggcctggaacgaaagctctgcatggctgctctgaaacaccagccacaggaattccctacctacgtggaacccacaaatgatgaaatctgtgaggcgttcaggaaagatccaaaggaatatgctaatcaatttatgtgggaatattccactaattacggacaagctcctctgtcacttttagtcagttacaccaagagttatctttctatggtagggtcctgctgtacctctgcaagcccaactgtatgctttttgaaagagagactccagcttaaacatttatcacttctcaccactctgtcaaatagagtctgctcacaatatgctgcttatggggagaagaaatcaaggctcagcaatctcataaagttagcccaaaaagtgcctactgctgatctggaggatgttttgccactagctgaagatattactaacatcctctccaaatgctgtgagtctgcctctgaagattgcatggccaaagagctgcctgaacacacagtaaaactctgtgacaatttatccacaaagaattctaagtttgaagactgttgtcaagaaaaaacagccatggacgtttttgtgtgcacttacttcatgccagctgcccaactccccgagcttccagatgtagagttgcccacaaacaaagatgtgtgtgatccaggaaacaccaaagtcatggataagtatacatttgaactaagcagaaggactcatcttccggaagtattcctcagtaaggtacttgagccaaccctaaaaagccttggtgaatgctgtgatgttgaagactcaactacctgttttaatgctaagggccctctactaaagaaggaactatcttctttcattgacaagggacaagaactatgtgcagattattcagaaaatacatttactgagtacaagaaaaaactggcagagcgactaaaagcaaaattgcctgatgccacacccacggaactggcaaagctggttaacaagcactcagactttgcctccaactgctgttccataaactcacctcctctttactgtgattcagagattgatgctgaattgaagaatatcctgtagtcctgaagcatgtttattaactttgaccagagttggagccacccaggggaatgatctctgatgacctaacctaagcaaaaccactgagcttctgggaagacaactaggatactttctactttttctagctacaatatcttcatacaatgacaagtatgatgatttgctatcaaaataaattgaaatataatgcaaaccataaaaaaaaaaaaaaaaaaaaaaa

SEQ ID NO: 95 - DNA, 2783 bp, Rattus norvegicus
  misc_feature (1)..(2783): cDNA for inter-alpha trypsin inhibitor, heavy chain 3
ctcagcaatggtgactctgtggtggccctgccttgtcttggctctgctctctggcttggagacctctggctttccaaggagtcccctccggctgcttgggaaacggagccttccagaaggggtggtggatggcattgagatctacagcaccaagatcagctgcaaggtgacctcccgctttgctcacaatgttgtcaccacaagggctgtcaaccgtgcagacaaggccaaggaagtttcctttgatgtggaactgcccaagacagccttcatcaccaacttcaccttgactattgatggtgtcacctaccctgggagtgtcaaggagaaggaagttgcgcagaaacaatatgaaaaggctgtgtcccagggcaagacagccggtttggtcaaggcctccgggaggaagctggagaagttcacagtgtctgtcaatgtggccgcaggcagcaaggtcatctttgagctgacctatgaagagctgctcaagagaaacaagggaaagtacgagatgtaccttaaggtccagcccaagcagctcgtcagacactttgagatcgacgcacacatttttgagccacagggcatcagcatgttggacgccgatgcctcatttatcactaatgacctcctgggaagtgccctcaccaagtccttctcagggaaaaaggggcatgtgtctttcaagcccagcttagaccagcagcgctcatgcccaacctgtacggactcccttctcaacggggacttcaccatcgtctatgatgtgaacagagagtctccaggaaatgtgcagatagtcaatggctacttcgtgcacttctttgcaccccaaggcctcccagtggtgcctaagaacatagcctttgtgattgacgtcagtggctccatgtctggtcggaaaatccagcagaccagggaagcccttctcaaaatcctggatgatatgaaggaagaggactatctgaatttcatcctcttcagcactggtgtgaccacctggaaagatcacctagtcaaggccacacctgcaaacctcgaggaggccagggcgtttgtgaagaacatccgtgatcgaagcatgaccaacatcaatgacgggctgctgaggggtattgaaatgctgaacaaggcccgagaagaccaccttgtcccggagaggagcacctccatcctcgtcatgttgacagatggggatgctaatactggcgagagcagacctgaaaagatccaggaaaacgtccggaacgccatcaggggcaagttccctttgtataacctgggttttggcaacaatctgaattataatttcctggagtctctggccctggagaaccacgggtttgcccggcgtatttatgaagattctgatgccagcttacagctgcagggcttctacgaggaagtagctaatcccttgttgactaatgtggagttagaataccctgagaatgccatcctggacctcaccagaaacagttacccccacttctatgatggctctgagattgttgtagcagggcgtctggtggacaggaacgtggacaacttcaaagcagatgtgaagggtcatggggccctgaatgacctgaccttcacagaggaggtagacatgaaggaaatggacgcggccctgaaggagcaaggctacatctttggggactacattgagcggctctgggcctacctcactattgagcagttactggagaaacgcaagaatgcccgtggggatgagaaggagaacatcacagcagaggccctggagctgtccctcaagtaccactttgtaactccactgacctccatggtggtgaccaagcctgaggacaacgaggaccaaacagccattgctgacaagcctggggaagaagccatctctgcgtccacggcctacctgaccagtcagcagtccagccatagcccctactattatgtggatggggacccccacttcatcatccaagtcccgggaaaaaatgacaccatctgcttcaacatcgatgagaagcctggcacggtgctgagccttattcaagacccagtcacaggcatcgcagtgaccgggcagatcattggagagaagggaaataatgcttcttccaggacagggaaaacttactttggcaaactgggcattgccaatgcttggatggactttcggattgaggtgaccacggagaagatcatcctggggaacggggatgcactgagcacgttcagttggctagacaccgtcacggtcacacagacagggctgtctgtgacaatcaacaggaaaaagaatatggtggtgtccttcgaagatgggattagctttgtgatcgtcctacaccaggtttggaagaaacatccggttcaccaggacttcctagggttctacgtggtggacagtcaccggatgtcagcacagacacatgggctgctgggccagttcttccaaccctttgactttaaagtgtttgacgtccgcccaggctctgaccccatgaagccagacgccacaatggtggtgaagagtcatcggttgacagtcacaaggggctcccaaaaagattacaggaaggatgccagtgtcggcaccaaggtcgtctgctggtttgtccataacaatggggaagggttcatcgacggtgtccacaccgactatattgtccccagcttgttctgagtacacatgccagctgctgctgcagtagaaggcctggaacaggacacagaatggggctcgtgggaaggactgggacaataaagtgggtaaaccaaaaaaaaaaaaaaaaaaa

SEQ ID NO: 96 - DNA, 3037 bp, Homo sapiens
  misc_feature (1)..(3037): cDNA for inter-alpha inhibitor H3
atataaggagcgtgggccaggcctgagtattcagcgatggcatttgcatggtggccctgtctcatcttggctctgctctccagcttggcagcctctggcttcccgagaagcccctttcggctgcttgggaaacggagcctcccggaaggggtggccaatggcatcgaggtctacagtaccaaaatcaactccaaggtgacctcccgttttgctcacaatgttgtcaccatgagagccgtcaaccgtgcagacacggccaaggaggtttcctttgatgtggagctgcccaagacggccttcatcaccaacttcaccttgaccatcgacggtgttacctaccctgggaatgtcaaggagaaggaagttgccaagaagcagtatgaaaaggctgtgtcccagggcaagacggccggcttggtcaaggcctctgggaggaagttggagaagttcacagtctcggtcaacgtggctgcaggcagcaaagtcaccttcgagctaacctacgaggagctgctgaagaggcacaagggcaagtacgagatgtacctcaaggtccagcctaagcaactggtcaaacactttgagatcgaggtagacatcttcgagcctcagggaatcagcatgctggatgctgaggcctctttcatcaccaacgacctcctgggaagcgccctcaccaagtccttctcagggaaaaagggccatgtgtccttcaagcccagcttagaccaacagcgttcatgcccaacctgtacagactccctcctcaatggagatttcactatcacctatgacgtgaacagagaatctcctggcaacgtgcagatagtcaatggctacttcgtgcacttctttgcacctcaaggccttccagtggtgcctaagaacgtggcctttgtgattgacatcagcggctccatggctggtcggaaattagagcagacaaaggaggcccttctcagaatcctggaagatatgcaagaggaagactatctgaatttcatcctgttcagtggagatgtgtccacatggaaagagcacttagtccaggccacgcccgagaacctccaggaggccaggacgtttgtgaagagcatggaggataaaggaatgaccaacatcaatgacgggctgctgaggggcatcagtatgctgaacaaggcccgagaggagcacagaatcccagagaggagcacctccattgtcatcatgctgactgatggggatgccaatgttggtgagagcagacccgaaaaaatccaagagaatgtgcggaatgccatcgggggcaagttccccttgtataacctgggctttggcaacaatctgaattataacttcctggagaacatggccctggagaaccatgggtttgcccggcgcatttatgaggactctgatgccgatttgcagttgcagggcttctatgaggaggtggccaacccactgctgacgggtgtggagatggagtaccccgagaacgctatcctggacctcacccagaacacttaccagcacttctacgatggctctgagatcgtggtggccgggcgcctggtggacgaggacatgaacagctttaaggcagatgtgaagggccatggggccaccaacgacctgaccttcacagaggaggtggacatgaaggagatggagaaggccctgcaggagcgggactacatcttcgggaattacattgagcggctctgggcctacctcaccattgagcagctgctggagaagcgcaagaacgcccatggcgaggagaaggagaacctcacggcccgggccctggacctgtccctcaagtatcactttgtgactccactgacctcaatggtggtgaccaagcctgaggacaacgaggatgagagggccattgccgacaagcctggggaagatgcagaagccacaccggtgagccccgccatgtcctacctgaccagctaccagcctcctcaaaacccctactactatgtggacggggatccccacttcatcatccaaattccggagaaagacgatgccctctgcttcaacatcgatgaagccccaggcacagtgctgcgccttattcaggatgcagtcacaggcctcacagttaatgggcagatcactggcgacaagagaggcagccctgactccaagaccagaaagacttactttggaaaactgggcatcgccaatgctcagatggacttccaggtggaggtgacaacggagaagatcaccctgtggaacagggccgtgccgagcactttcagctggctggacacagtcacagtcacgcaggatgggctgtccatgatgatcaacaggaagaacatggtggtctcctttggagatggggttaccttcgtggtcgtcctacaccaggtgtggaagaaacatcctgtccaccgtgactttctaggcttctacgtggtggacagtcaccggatgtcagcacagacgcatgggctgctggggcaattcttccaaccctttgactttaaagtgtctgacatccggccaggctctgaccccacaaagccagatgccacattggtggtgaagaaccatcagctgattgtcaccaggggctcccagaaagactacagaaaggatgccagcatcggcacgaaggttgtctgctggttcgtccacaacaacggagaagggctgattgatggtgtccacactgactacattgtccccaacctgttttgagtagacacaccagctcctgttgggatggatggcccggattttatggcatctggaacatgggcacagagaggggcctgtgggaggggctgggaaaataaagtccaaggtcgagaccagacagctgccagcctctacatgccttcatgagcctctgctccagggaacaggcaactgggacccacagcagctcaaaagaggggatggctagcaggaaggcaacttcttgggcctctccactgacacagctgagagacagagttggttggactagaactttagggattcaaatgtatgacagcttgggggaaaaaataaagaactgactctg

SEQ ID NO: 97 - DNA, 1721 bp, Rattus norvegicus
  misc_feature (1)..(1721): cDNA for cytochrome P450, family 2, subfamily c, polypeptide 13
gaagggtctccatggatccagttgtggtcttgttgctcagtctgttctttctgcttttcctgtctctatggagactgagctctgggagagggaaactccctcctggcccaactcctctcccaattattggaaatttctttcaggttgatatgaaggacattcggcaatccttaaccaatttttctaaaacctatggacctgtgtatactctgtatgttggctcacagcctactgtggtattgcatggctatgaggcattgaaagaagctcttgttgatcatggggaagaattctctggaagaggaaggttaccaatttgtgaaaaggttgctaaaggacagggcattgcttttagccatggaaatgtatggaaagccacaaggcatttcacagtcaaaaccctgaggaatttgggcatgggaaaagggaccattgaagacaaagtgcaagaggaagcaaagtggctagtgaaagaactgaagaaaaccaatggctcaccctgtgatccccaattcatcatgggatgtgctcctggcaatgtcatctgctgcattatcttgcagaatcgttttgattatgaagataaggattttcttaacttgatagaaaaagtgaatgaggccgttaaaattataagctctcctgggattcaggttttcaatatatttcctatacttcttgattattgtccgggaaatcataacatatatttaaaaaattatacatgggttaagagttaccttttggagaaaataaaagaacatgaagaatcgttggatgtttcaaatcctcgggacttcattgattattttctaattgaaagaaatcaggaaaatgccaatcagtggatgaactatacacttgaacacctggcaatcatggtgactgatttgttttttgctggaatagagacagtaagctcaacaatgagatttgctctcctgctcttgatgaagtacccacacgtcacagctaaagtccaggaagagattgaccatgtgattgggagacaccgcagcccctctatgcaggacaggagccacatgccctacacaaatgccatggtccacgaggttcagagatacattgacattggtccaaatggtctgcttcatgatgtgacctgtgacactaaattcagaaactacttcataccgaagggaacagcagtactaacatcacttacatcagtgctgcatgacagcaaggagtttcccaacccagagatgtttgacccaggtcactttctagatgagaatggaaactttaagaaaagtgactacttcattcctttctcagcaggaaaacggatgtgtttgggagagagcctggcccgcatggagctgtttctattcctgacaaccattttacagaatttcaaactaaaatctctggttgacccaaaggacatcaatacaaccccaatatgttcttcactttcttcagttcctcccactttccagatgcgcttcatccctctctaatgaaaaccagaaaacagaaatgaagatgaggaaagatcctgctgttttgttttctgaaggtacccacagaagccttcatttactactgtcatagaaatgccactgtatccttccttcttctcacagcttcggttctctatttgtccaaggataatagttctgcattatatagttcctaaactcaatgtaaaagatcctgataaagaagccacttcaaataaaagagttctactgtga

SEQ ID NO: 98 - DNA, 1175 bp, Rattus norvegicus
  misc_feature (1)..(1175): cDNA for apolipoprotein H
catctacaatgatttctccggcgctcatcttcttttctgccttcctctgccatgttgctattgcgggacggacctgtccgaagccggatgaactaccgtttgctgtggttgttcccttaaaaacattctatgacccaggggagcagattgtctattcctgcaagccaggctatgtgtccaggggagggatgagacggtttacctgtcctctcacagggatgtggcccatcaacaccctgaaatgtatacccagagtgtgtccttttgccggaatcttagaaaatggagttgtacgctacacgacttttgaatatcccaacaccattggctttgcttgtaaccctgggtattatctgaacgggactagctcttctaagtgcacggaggaaggaaaatggagtccagaacttcctgtttgtgctcggatcacctgcccgccaccaccaattcctaagtttgcagcccttaaggaatacaagacttcagttgggaacagctctttttatcaggacacagtggtctttaaatgcttgccacactttgccatgtttggaaatgacacagttacgtgcacagcccatggaaactggacccaattgccagaatgccgggaagtaaaatgtcccttcccttcaaggccagacaatgggtttgtgaattaccctgcaaagccggtgctttcttataaagataaagccgtatttggttgccatgaaacatacaaactggatggcccggaagaagtggagtgtaccaagacaggaaattggtcggccctgccgagctgtaaagcatcttgcaaactctctgttaagaaggccaccgtgttgtaccaagggcagagggtgaagatccaggaccagtttaagaatgggatgatgcatggtgacaaggttcacttctattgtaaaaataaggagaagaagtgcagctacaccgaggaggctcagtgcatagacggcaccatcgagatccccaaatgcttcaaggagcacagttctctggctttctggaaaacggatgcatcagatgtgacaccgtgctgaagacattttcagaatcaaaattgaatgtctcatttgtatctctgcttgtctgaggaatcaactgaaatagcagaataaactaccgagttgcctcagtcaaaaaaaaaaaaaaaaaaaaaaaaaaaaaaa

SEQ ID NO: 99 - DNA, 1216 bp, Homo sapiens
  misc_feature (1)..(1216): cDNA for apolipoprotein H
ggctctgtctttttagcagacgaaaaccactttggtagtgccagtgtgactcatccacaatgatttctccagtgctcatcttgttctcgagttttctctgccatgttgctattgcaggacggacctgtcccaagccagatgatttaccattttccacagtggtcccgttaaaaacattctatgagccaggagaagagattacgtattcctgcaagccgggctatgtgtcccgaggagggatgagaaagtttatctgccctctcacaggactgtggcccatcaacactctgaaatgtacacccagagtatgtccttttgctggaatcttagaaaatggagccgtacgctatacgacttttgaatatcccaacacgatcagtttttcttgtaacactgggttttatctgaatggcgctgattctgccaagtgcactgaggaaggaaaatggagcccggagcttcctgtctgtgctcccatcatctgccctccaccatccatacctacgtttgcaacacttcgtgtttataagccatcagctggaaacaattccctctatcgggacacagcagtttttgaatgtttgccacaacatgcgatgtttggaaatgatacaattacctgcacgacacatggaaattggactaaattaccagaatgcagggaagtaaaatgcccattcccatcaagaccagacaatggatttgtgaactatcctgcaaaaccaacactttattacaaggataaagccacatttggctgccatgatggatattctctggatggcccggaagaaatagaatgtaccaaactgggaaactggtctgccatgccaagttgtaaagcatcttgtaaagtacctgtgaaaaaagccactgtggtgtaccaaggagagagagtaaagattcaggaaaaatttaagaatggaatgctacatggtgataaagtttctttcttctgcaaaaataaggaaaagaagtgtagctatacagaggatgctcagtgtatagatggcactatcgaagtccccaaatgcttcaaggaacacagttctctggctttttggaaaactgatgcatccgatgtaaagccatgctaaggtggttttcagattccacacaaaatgtcacacttgtttcttgttcatccaaggaacctaattgaaatttaaaaataaagctactgaatttattgccgcacccaaaaaaaaaaaaaaaa

SEQ ID NO: 100 - DNA, 1561 bp, Rattus norvegicus
  misc_feature (1)..(1561): cDNA for serpin peptidase inhibitor, clade C, member 1
cggagggattgctcagcactgtctccacggcttctctgcagaagcgtccaccatgtattccccgggaataggaagtgcggttgctggagagaggaagctttgtctcctctctctgctactcattggtgccttgggctgtgctgtctgtcatggaaaccctgtggacgacatctgcatagcgaagccccgagacatccccgtgaaccccatgtgcatttaccgctcccctgcgaagaaggccacggaggaggatgtcctagagcagaaggttccggaagccaccaaccggcgggtctgggaactgtccaaggccaattctcgatttgccactaacttctatcagcacctggcagactccaagaacgacaacgacaacattttcctgtcacccttgagcatctccacggcgtttgctatgaccaagctgggtgcttgtaataacaccctcaagcagctgatggaggtttttaaatttgataccatctccgagaagacatccgaccagatccacttcttctttgccaaactgaactgccgactctatcgaaaagccaacaagtcctctaacttggtgtcagccaaccgcctttttggagacaaatcccttaccttcaatgagagctatcaagacgttagtgagattgtctatggagccaagcttcagcccctggacttcaaggagaatccggagcaatccagagtgaccatcaacaactgggtagctaataagactgaaggccgcatcaaagacgtcatcccccaaggagccattgatgagctcactgccctggtgctggttaacaccatttacttcaagggcctgtggaagtcaaagttcagccctgagaacacaaggaaggaaccattccacaaagttgatgggcagtcatgcctggtgcccatgatgtaccaggaaggcaaattcaaatacaggcgtgtgggagagggtacccaggtgctagagatgcccttcaagggggacgacatcaccatggtgctcatcctgcccaagcctgagaagagcctggctaaggtggagcaggaactcaccccggagctgctgcaggagtggctggatgagctgtcggaggtcatgcttgtggtccacgtgccccgcttccgcatcgaggacagcttcagtctgaaggagcagctgcaagacatgggccttgttgatctcttcagccctgagaagtcccaactcccagggatcattgctgaaggcagggacgacctctttgtctccgatgcattccacaaagcgtttcttgaggtaaatgaggaaggcagtgaagcagcagcgagtacttctgtcgtgattactggccggtcactgaaccccagtagggtgaccttcaaggccaacaggcccttcctggttcttataagggaagtcgcactgaacactattatattcatggggagagtgtctaatccttgtgtgaactaaaatattcttaatctttgcaccttttcctatctcggtgtttgttaatggaagtaaaaataaatatgactgccacctcaaaaaaaaaaaaaaaaaaaaaaaaaaaaaaaaaaa

SEQ ID NO: 101 - DNA, 1599 bp, Homo sapiens
  misc_feature (1)..(1599): cDNA for serpin peptidase inhibitor, clade C, member 1
tctgccccaccctgtcctctggaacctctgcgagatttagaggaaagaaccagttttcaggcggattgcctcagatcacactatctccacttgcccagccctgtggaagattagcggccatgtattccaatgtgataggaactgtaacctctggaaaaaggaaggtttatcttttgtccttgctgctcattggcttctgggactgcgtgacctgtcacgggagccctgtggacatctgcacagccaagccgcgggacattcccatgaatcccatgtgcatttaccgctccccggagaagaaggcaactgaggatgagggctcagaacagaagatcccggaggccaccaaccggcgtgtctgggaactgtccaaggccaattcccgctttgctaccactttctatcagcacctggcagattccaagaatgacaatgataacattttcctgtcacccctgagtatctccacggcttttgctatgaccaagctgggtgcctgtaatgacaccctccagcaactgatggaggtatttaagtttgacaccatatctgagaaaacatctgatcagatccacttcttctttgccaaactgaactgccgactctatcgaaaagccaacaaatcctccaagttagtatcagccaatcgcctttttggagacaaatcccttaccttcaatgagacctaccaggacatcagtgagttggtatatggagccaagctccagcccctggacttcaaggaaaatgcagagcaatccagagcggccatcaacaaatgggtgtccaataagaccgaaggccgaatcaccgatgtcattccctcggaagccatcaatgagctcactgttctggtgctggttaacaccatttacttcaagggcctgtggaagtcaaagttcagccctgagaacacaaggaaggaactgttctacaaggctgatggagagtcgtgttcagcatctatgatgtaccaggaaggcaagttccgttatcggcgcgtggctgaaggcacccaggtgcttgagttgcccttcaaaggtgatgacatcaccatggtcctcatcttgcccaagcctgagaagagcctggccaaggtagagaaggaactcaccccagaggtgctgcaagagtggctggatgaattggaggagatgatgctggtggtccacatgccccgcttccgcattgaggacggcttcagtttgaaggagcagctgcaagacatgggccttgtcgatctgttcagccctgaaaagtccaaactcccaggtattgttgcagaaggccgagatgacctctatgtctcagatgcattccataaggcatttcttgaggtaaatgaagaaggcagtgaagcagctgcaagtaccgctgttgtgattgctggccgttcgctaaaccccaacagggtgactttcaaggccaacaggcctttcctggtttttataagagaagttcctctgaacactattatcttcatgggcagagtagccaacccttgtgttaagtaaaatgttcttattctttgcacctcttcctatttttggtttgtgaacagaagtaaaaataaatacaaactacttccatctcacatta

SEQ ID NO: 102 - DNA, 1664 bp, Rattus norvegicus
  misc_feature (1)..(1664): cDNA for serine peptidase inhibitor, clade A, member 3K
aacacaccagggcaccctgaacatcaggagtcggcaaacacagaggctagtagctggctggtatcacctctgcagccaggaggacagagaagatggccttcattgcagctttggggctcttgatggcagggatctgccctgctgtcctctgtgatggcatactgggaagggacactctaccccatgaagaccaaggcaaggggagacaactgcacagtctcacattggcttccatcaacactgacttcacattgagcctctacaagaagctggctttgaggaatccagataaaaatgttgtcttctcccctcttagcatctcagctgccttggccattttgtctctgggagcaaaagacagcaccatggaagagattctagaagtactcaagttcaatctcacagagatcactgaggaagaaatccaccagggctttgggcacctcctacagaggctcagccagccagaggaccaggcagagatcaatacaggtagtgccctgtttattgacaaagagcagccgatactgtcagaattccaggagaagacaagggctctgtaccaggctgaggccttcgtagctgacttcaagcagtgcaatgaggccaaaaagttcatcaatgactatgtgagcaatcagacccaggggaagatcgcagaactgttctcagaactggatgagaggacatccatggtgctggtgaactatctcctctttaaaggcaaatggaaggtaccatttaaccccaatgacacatttgagtctgagttctacttggatgagaagaggtctgtgaaggtgcccatgatgaaaattaaggatctcaccacaccctacatccgggatgaggagctgtcctgctctgtgctggaactgaagtacacaggaaatgccagcgccctgtttatcctccctgaccagggcaagatgcagcaggtggaatccagcttgcaaccagagaccctgaagaagtggaaggactctctgaggcccaggattataagtgagcttcgcatgcccaagttctccatctccacagactacaacctggaggaggtccttccagacctgggaattaggaaaatcttctcccagcaagctgatctgagtaggatcacagggaccaagaacctgcatgtctctcaggtggtccacaaagctgtgctggatgtggatgagacaggcacagaaggagccgctgccacagcagtcacagcagccctaaaaagtttaccgcaaactatacctcttctgaatttcaaccggccattcatgctggttatcactgacaataatggtcagtctgtcttctttatgggcaaagtcactaaccccatgtgatctgaagctccccaaaatctgacaattctgcccaggatcctggaacagagcctggatgctgatctctgtatatgccctgacatacatgctctgattggctattgcaaagttggcttagacagtgacatcaactatctctatggctcccatgtgcactggagcctttggattgtcagtgtgcaggcacttaggacccttgggagcatctacacatgtttctgaacttggaatctttctttattcttcttccctggtgactcctctttctgtgttcataccccaaaccaagccattgataagtcccagtaaaggttctgagaa

SEQ ID NO: 103 - DNA, 431 bp, Rattus norvegicus
  misc_feature (1)..(431): cDNA for apolipoprotein C-I
gcgggcctcctgagagaaccttagatccagggtgcccctccagccaggatgaggctcttcatcgcacttcctgtcctgatcgtggtcgtagccatggctttggaaggcccagcccctgcccaggcggcccctgacttttccagcgcaatggagagcttaccggataaactaaaagagtttgggaacactttggaagataaggcccgggcagccattgagcatatcaaacagaaggaaattatgatcaagactcggaactggttttcagagacgctcaacaaaatgaaggagaaattaaaaaccacatttgcctgagtgcctggcgagccacctcgcagcagcaaagagggccacgtgacgggccaggtccctcttcaaataccaataaaaacttgtagagaaaaaaaaaaaaaaaaaaaaaaaaaaaaaaa

SEQ ID NO: 104 - DNA, 464 bp, Homo sapiens
  misc_feature (1)..(464): cDNA for apolipoprotein C-I
ctccccagcctgataaaggtcctgcgggcaggacaggacctcccaaccaagccctccagcaaggattcagagtgcccctccggcctcgccatgaggctcttcctgtcgctcccggtcctggtggtggttctgtcgatcgtcttggaaggcccagccccagcccaggggaccccagacgtctccagtgccttggataagctgaaggagtttggaaacacactggaggacaaggctcgggaactcatcagccgcatcaaacagagtgaactttctgccaagatgcgggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaattcaaa

SEQ ID NO: 105 - DNA, 947 bp, Rattus norvegicus
  misc_feature (1)..(947): cDNA for secreted phosphoprotein 2
attgacacgggaatcaatcatctctactgaggttccagactgcttcagccagccagggcagctaggtcgcaggtgacaaggataagacagtcaccctcggaaagagctgtagagaggccatctccttggctctgggtcgccgtcctcgggatggagctggcaacgatgaagacgctggttatgttggtgctgggaatgcactactggtgtgcctcaggtttcccggtgtacgactacgacccttcctctctgcaagaagctctcagtgcctcagtggctaaggtgaactcgcaatccctgagtccttacctgtttcgggcgacgcggagctccctgaagagagttaatgtcctggatgaagacacactggtcatgaacttagagttcaccgttcaggaaactacatgcctaagagagtctggtgatccctccacctgcgccttccaaaggggctactctgtgccaacagctgcttgcaggagcacagtgcagatgtccaagggacaggtgaaggatgtgtgggctcactgccgctgggcgtccacatccgagtccaacagcagtgaggagatgatttttggggacatggcaagatcccaccgacgaagaaatgattatctacttggctttctttatgatgaacccaaaggtgagcagttctatgaccggtcgattgagatcacgaggaggggacatcctcctgcccatagaaggttcctgaacctccaacgcagagcaagagtcaattctggctttgagtgacatcctggagattccatgaaagaaagagaagcagaagctgaaatgaaggacatggagaattgtgtctttttcctttgataagctccactttgcaataaagatcttccccttcctgtaaaaaaaaaaaaaaaaaaaaaaaaaaaaaaaaaaaaaaaaaaaaaaaaaaaaaaaaaaaaaaaaaaaaa

SEQ ID NO: 106 - DNA, 1019 bp, Homo sapiens
  misc_feature (1)..(1019): cDNA for secreted phosphoprotein 2
agtgtttgataaagacagctcctcttaggaagaactgtcatccccaaacacatagagagacactctctgtctctcgattacaatcatgatttccagaatggagaagatgacgatgatgatgaagatattgattatgtttgctcttggaatgaactactggtcttgctcaggtttcccagtgtacgactacgatccatcctccttaagggatgccctcagtgcctctgtggtaaaagtgaattcccagtcactgagtccgtatctgtttcgggcattcagaagctcattaaaaagagttgaggtcctagatgagaacaacttggtcatgaatttagagttcagcatccgggagactacatgcaggaaggattctggagaagatcccgctacatgtgccttccagagggactactatgtgtccacagctgtttgcagaagcaccgtgaaggtatctgcccagcaggtgcagggcgtgcatgctcgctgcagctggtcctcctccacgtctgagtcttacagcagcgaagagatgatttttggggacatgttgggatctcataaatggagaaacaattatctatttggtctcatttcagacgagtccataagtgaacaattttatgatcggtcacttgggatcatgagaagggtattgcctcctggaaacagaaggtacccaaaccaccggcacagagcaagaataaatactgactttgagtaacggccttgaggtgtccctcgcccttttggtttgttcaaggagctgctgctttgcatagctgctctagtgtctggtatcatcggatctggttttgaataattcccaggagtcctgggtccctggcctccaaagctggaatgtgaacgcatgccacggtggtctgaccctcacactccttttctcttaacagcaaaatgcaatggaaggaagaaaagttccaacaaagaatgattttgtgaattctgtgatttttcttctgatcagtttcaatctgtaataaatgccttatttttcctgt